(19) United States
(12) Patent Application Publication
(10) Pub. No.: US 2026/0286376 A1
(43) Pub. Date: Sep. 24, 2026

(54) ENGINEERING INCREASED SUBERIN LEVELS IN PLANTS

(71) Applicant: Salk Institute for Biological Studies, La Jolla, CA (US)

(72) Inventors: Julie Ann Law, La Jolla, CA (US); Mina Rostamza, La Jolla, CA (US); Joseph P. Noel, La Jolla, CA (US); Suzanne Thomas, La Jolla, CA (US); Jeffrey Todd Mindrebo, La Jolla, CA (US); Luke Alden Sarre, La Jolla, CA (US)

(73) Assignee: Salk Institute for Biological Studies, La Jolla, CA (US)

(21) Appl. No.: 19/477,056

(22) PCT Filed: Apr. 19, 2024
(86) PCT No.: PCT/US2024/025564

§ 371 Date: Oct. 20, 2025

Related U.S. Application Data
(60) Provisional application No. 63/497,697, filed on Apr. 21, 2023.

(51) Int. Cl. C12N 15/82; C07K 14/415

(52) U.S. Cl. CPC C12N 15/8247; C07K 14/415

(57) ABSTRACT

The present disclosure provides compositions and methods for increasing suberin production in plants, for example by increasing MYB41 expression in a plant. In some examples, MYB41 expression is by a heterologous promoter, such as a FACT gene promoter, a HORST gene promoter, a GPAT5 gene promoter, a RALPH gene promoter, a ASFT gene promoter, or a MYB84 gene promoter.

### CROSS REFERENCE TO RELATED APPLICATIONS

This application claims priority to U.S. Provisional Application No. 63/497,697, filed Apr. 21, 2023, which is incorporated by reference in its entirety.

### FIELD

The present disclosure generally relates to the field of increasing suberin production in plants. More particularly, the present disclosure relates to compositions and methods for generating plants that possess increased amounts of periderm and/or increased amounts of suberin in their roots as compared to control plants. Also, the present disclosure relates to the enhanced root cell-type specific expression of genetic materials related to suberin synthesis and suberization.

### INCORPORATION OF ELECTRONIC SEQUENCE LISTING

The Sequence Listing is submitted as an XML file in the form of the file named “7158-110706-02_ST26.xml” (~1,025,763 bytes), which was created on Apr. 17, 2024 which is incorporated by reference herein.

### BACKGROUND OF THE DISCLOSURE

In plant roots, suberin is deposited in specific locations, including the periderm, where it serves as a barrier between the plant and its environment (Vishwanath et al., 2015). The suberin molecule contains vast amounts of carbon in forms that are thought to persist for long periods of time in the soil (Carrington et al., 2012; Feng and Simpson, 2011; Preston et al., 1997; Winkler et al., 2005). Thus, by increasing suberin levels in roots, more carbon can be sequestered from the atmosphere, through the act of photosynthesis, and stored for long periods of time as a means to mitigate climate change. However, global overexpression of suberin regulators using 35S promoters are known to negatively impact plant health (Mahmood et al., 2019).

There is a need to identify regulators for suberin biosynthesis and suberization and manipulate gene expression of the regulators to produce genetically-modified or gene-edited plants that develop additional periderm layers at root development and/or deposit more suberin in periderm cells without negatively impacting plant health.

### SUMMARY OF THE DISCLOSURE

The present disclosure solves the aforementioned goal of increasing suberin production in plants by identifying promoters that express in specific root tissues, such as in the phellogen, pericycle or procambium. Furthermore, the disclosure includes methodology by which this ability to increase suberin can be imported into any plant genus or species. The importation of this genetic architecture can take many forms, as elaborated upon herein, including: traditional plant breeding, transgenic genetic engineering, next generation plant breeding (CRISPR, base editing, MAS, etc.), and other methods.

In some examples, the present disclosure provides isolated nucleic acid molecules encoding a MYB41 amino acid sequence with at least 80% sequence homology to any one of SEQ ID NOs: 37-91 and/or a nucleic acid set forth in any one of SEQ ID NOs: 92-201, operably linked to a nucleic acid sequence encoding a heterologous promoter, wherein expression of the isolated nucleic acid molecule in a plant results in increased levels of suberin as compared to wild-type check plants lacking the isolated nucleic acid molecule. In some examples of the present disclosure, the increased levels of suberin occur by generating additional periderm cells and/or depositing more suberin in existing periderm cells.

In some examples of the present disclosure, the isolated nucleic acid molecules have amino acid sequence of at least 85% homology, at least 90% homology, at least 95% homology, at least 96% homology, at least 97% homology, at least 98% homology and at least 99% homology to any one of SEQ ID NOs: 37-91. In some examples of the present disclosure, the isolated nucleic acid molecules have an amino acid sequence homology that is 100% homologous to any one of SEQ ID NOs: 37-91.

In some examples of the present disclosure, the isolated nucleic acid molecules have nucleic acid sequence of at least 85% homology, at least 90% homology, at least 95% homology, at least 96% homology, at least 97% homology, at least 98% homology or at least 99% homology to any one of SEQ ID NOs: 92-201.

In some examples, the MYB41 amino acid sequence is present in Canola (Brassica napus) and comprises any one of SEQ ID NOs: 37-48 or a sequence at least 80%, at least 90%, at least 95%, at least 98%, or at least 99% identical thereto. In some examples, the MYB41 amino acid sequence is present in Soybean (Glycine max) and comprises any one of SEQ ID NOs: 49-56 or a sequence at least 80%, at least 90%, at least 95%, at least 98%, or at least 99% identical thereto. In some examples, the MYB41 amino acid sequence is present in Cotton (Gossypium hirsutum) and comprises any one of SEQ ID NOs: 57-68 or a sequence at least 80%, at least 90%, at least 95%, at least 98%, or at least 99% identical thereto. In some examples, the MYB41 amino acid sequence is present in Rice (Oryza sativa) and comprises any one of SEQ ID NO: 69 or a sequence at least 80%, at least 90%, at least 95%, at least 98%, or at least 99% identical thereto. In some examples, the MYB41 amino acid sequence is present in Lotus (Lotus japonicus) and comprises any one of SEQ ID NOs: 70-72 or a sequence at least 80%, at least 90%, at least 95%, at least 98%, or at least 99% identical thereto. In some examples, the MYB41 amino acid sequence is present in Radish (Raphanus sativus) and comprises any one of SEQ ID NOs: 73-81 or a sequence at least 80%, at least 90%, at least 95%, at least 98%, or at least 99% identical thereto. In some examples, the MYB41 amino acid sequence is present in Setaria (Setaria italica) and comprises any one of SEQ ID NO: 82 or a sequence at least 80%, at least 90%, at least 95%, at least 98%, or at least 99% identical thereto. In some examples, the MYB41 amino acid sequence is present in Sorghum (Sorghum bicolor) and comprises any one of SEQ ID NO: 83 or a sequence at least 80%, at least 90%, at least 95%, at least 98%, or at least 99% identical thereto. In some examples, the MYB41 amino acid sequence is present in Pennycress (Thlaspi arvense) and comprises any one of SEQ ID NOs: 84-86 or a sequence at least 80%, at least 90%, at least 95%, at least 98%, or at least 99% identical thereto. In some examples, the MYB41 amino acid sequence is present in Southern cattail (Typha domingensis) and comprises any one of SEQ ID NO: 87 or a sequence at least 80%, at least 90%, at least 95%, at least 98%, or at least 99% identical thereto. In some examples, the MYB41 amino acid sequence is present in Wheat (Triticum aestivum) and comprises any one of SEQ ID NOs: 88-89 or a sequence at least 80%, at least 90%, at least 95%, at least 98%, or at least 99% identical thereto. In some examples, the MYB41 amino acid sequence is present in Maize (Zea mays) and comprises any one of SEQ ID NOs: 90-91 or a sequence at least 80%, at least 90%, at least 95%, at least 98%, or at least 99% identical thereto.

In some examples, the MYB41 amino acid sequence comprises at least 80%, at least 90%, at least 95%, at least 98%, at least 99%, or 100% sequence identity to any one of SEQ ID NOs: 38, 40, 44, 46, 49, 52, 70, 76, 77, 78, and 86. In some examples, the nucleic acid sequence comprises at least 80%, at least 90%, at least 95%, at least 98%, at least 99%, or 100% sequence identity to any one of SEQ ID NOs: 93, 95, 99, 101, 104, 107, 125, 131, 132, 133, and 141. In some examples, the nucleic acid sequence comprises at least 80%, at least 90%, at least 95%, at least 98%, at least 99%, or 100% sequence identity to any one of SEQ ID NOs: 148, 150, 154, 156, 159, 162, 180, 186, 187, 188, and 196.

In some examples of the present disclosure, the heterologous promoter comprises an isolated nucleic acid sequence set forth in any one of SEQ ID NO: 1, SEQ ID NO: 2, SEQ ID NO: 3, SEQ ID NO: 4, SEQ ID NO: 5, and SEQ ID NO: 6, or a sequence at least 70%, at least 80%, at least 90%, at least 95%, at least 98%, or at least 99% identical thereto. In some examples, the heterologous promoter is a native promoter of the FACT gene. In some examples, the heterologous promoter is a native promoter of the HORST gene. In some examples, the heterologous promoter is a native promoter of the ASFT gene. In some examples, the heterologous promoter is a native promoter of the GPAT5 gene. In some examples, the heterologous promoter is a native promoter of the RALPH gene. In some examples, the heterologous promoter is a native promoter of the MYB84 gene. In some aspects, the heterologous promoter comprises an isolated nucleic acid sequence set forth in any one of SEQ ID NOs: 233-411, or a sequence at least 70%, at least 80%, at least 90%, at least 95%, at least 98%, or at least 99% identical thereto. In some examples, the promoter is a promoter of FACT gene, such as an isolated nucleic acid sequence set forth in any one of SEQ ID NOs: 1, 234, 239-246, 280-285, 314-316, 340-349, 379, 380, or 389-391, or a sequence at least 70%, at least 80%, at least 90%, at least 95%, at least 98%, or at least 99% identical thereto. In some examples, the promoter is a promoter of HORST gene, such as an isolated nucleic acid sequence set forth in any one of SEQ ID NOs: 2, 236, 254-258, 288, 289, 319-321, 352-354, 383, 394, or 395, or a sequence at least 70%, at least 80%, at least 90%, at least 95%, at least 98%, or at least 99% identical thereto. In some examples the promoter is a promoter of ASFT gene, such as an isolated nucleic acid sequence set forth in any one of SEQ ID NOs: 3, 233, 239-244, 280-285, 314-316, 340-349, 378, 379, or 389-391, or a sequence at least 70%, at least 80%, at least 90%, at least 95%, at least 98%, or at least 99% identical thereto. In some examples, the promoter is a promoter of GPAT5 gene, such as an isolated nucleic acid sequence set forth in any one of SEQ ID NOs: 4, 235, 247-253, 286, 287, 317, 318, 350, 351, 381, 382, 392, or 393, or a sequence at least 70%, at least 80%, at least 90%, at least 95%, at least 98%, or at least 99% identical thereto. In some examples the promoter is a promoter of RALPH gene, such as an isolated nucleic acid sequence set forth in any one of SEQ ID NOs: 5, 238, 387, or 388, or a sequence at least 70%, at least 80%, at least 90%, at least 95%, at least 98%, or at least 99% identical thereto. In some examples promoter is a promoter of MYB84 gene, such as an isolated nucleic acid sequence set forth in any one of SEQ ID NOs: 6, 237, 259-279, 290-313, 322-339, 355-377, 384-386, or 396-411, or a sequence at least 70%, at least 80%, at least 90%, at least 95%, at least 98%, or at least 99% identical thereto.

In some examples as provided herein are transformation vectors comprising one or more of the nucleic acid molecules of the present disclosure.

In some examples as provided herein are methods of transforming plant cells. In some aspects, such methods include introducing one or more transformation vectors of the present disclosure into the plant cells, thereby generating a transformed plant cell, whereby the transformed plant cells produce increased levels of suberin as compared to an untransformed wild-type check plant cell. In some examples of the present disclosure, the methods further include producing transformed plant tissues from the transformed plant cells. In some examples of the present disclosure, the methods further include producing a transformed plantlet from the transformed plant tissue, wherein the transformed plantlet produces increased levels of suberin as compared to untransformed wild-type check plantlets lacking the isolated nucleic acid molecule.

In some aspects, the methods further include producing a progeny of the transformed plantlet, wherein the progeny produces increased levels of suberin as compared to untransformed wild-type check plantlets lacking the isolated nucleic acid molecule.

In some aspects, the methods include growing the transformed plantlet or the progeny of the transformed plantlet into a mature transformed plant, wherein the mature transformed plant produces increased levels of suberin as compared to mature untransformed wild-type checks lacking the isolated nucleic acid molecule. In some examples, the methods provided herein result in increased levels of suberin that occur by generating additional periderm cells and/or depositing more suberin in existing periderm cells. In some examples, the methods provided herein result in minimal or no expression of the nucleic acid molecule in cells that are not associated with normal suberin production. In some examples, the methods result in minimal or no expression of the nucleic acid molecule in rosette leaves.

In some aspects, the methods further include using the mature transformed plant or clone of the mature transformed plant in a breeding method. In some examples, the breeding methods comprise selfing or crossing the mature transformed plant or clone of the mature transformed plant.

In some aspects, the plant breeding methods include crossing a first plant comprising a nucleic acid molecule of the present disclosure with a second plant of the same species and selecting resultant progeny of the cross based on increased levels of suberin as compared to wild-type check plants. In some examples, the plant breeding methods further include producing clones of the resultant progeny of the cross wherein the clones are selected based on increased levels of suberin as compared to wild-type check plants. In some examples, the progeny of the cross that display increased levels of suberin as compared to wild-type check plants are selected using molecular markers that are designed based on the nucleic acid molecule of the present disclosure. In some examples, the methods further comprise using the selected progeny in a breeding method.

In some aspects, the methods further include growing the mature transformed plant or clone of the mature transformed plant in a greenhouse or outdoors. In some examples, the outdoor growing may be in a farm field, a marshland, a plant nursery, rangeland, prairie land, open space, forest land and timber production.

In some examples of the present disclosure, a plant, a plant part or a plant cell thereof, comprises the isolated nucleic acid molecule or the transformation vector taught herein. In some examples of the present disclosure, a plant, a plant part or a plant cell thereof, produced by the methods taught herein.

In some examples of the present disclosure, a genetically engineered plant includes an isolated nucleic acid molecule encoding a MYB41 amino acid sequence that is selected from the group consisting of any one of SEQ ID NOs: 37-91 or an isolated nucleic acid molecule that is selected from the group consisting of any one of SEQ ID NOs: 92-201.

In some examples of the present disclosure, a genetically engineered plant includes an isolated nucleic acid molecule encoding a MYB41 amino acid sequence selected from the group including SEQ ID NOs: 38, 40, 44, 46, 49, 52, 70, 76, 77, 78, and 86, operably linked to a nucleic acid sequence encoding a heterologous promoter, where expression of the isolated nucleic acid molecule in a plant results in increased levels of suberin as compared to wild-type check plants lacking the isolated nucleic acid molecule.

In some examples of the present disclosure, a genetically engineered plant, includes an isolated nucleic acid molecule that is selected from the group including SEQ ID NOs: 93, 95, 99, 101, 104, 107, 125, 131, 132, 133, 141 148, 150, 154, 156, 159, 162, 180, 186, 187, 188, and 196, operably linked to a nucleic acid sequence encoding a heterologous promoter, where expression of the isolated nucleic acid molecule in a plant results in increased levels of suberin as compared to wild-type check plants lacking the isolated nucleic acid molecule.

In some examples of the present disclosure, the genetically engineered plant is Canola (Brassica napus), Soybean (Glycine max), Cotton (Gossypium hirsutum), Rice (Oryza sativa), Lotus (Lotus japonicus), Radish (Raphanus sativus), Setaria (Setaria italica), Sorghum (Sorghum bicolor), Pennycress (Thlaspi arvense), Southern cattail (Typha domingensis), Wheat (Triticum aestivum), or Maize (Zea mays).

In some examples of the present disclosure, the heterologous promoter is a promoter of FACT gene. In some examples, the promoter is operably linked to the isolated nucleic acid molecule encoding a MYB41 amino acid sequence selected from the group including SEQ ID NOs: 38, 40, 44, 46, 49, 52, 70, 76, 77, 78, and 86. In some examples, the heterologous promoter includes an isolated nucleic acid sequence set forth in any one of SEQ ID NOs: 1, 234, 239-246, 280-285, 314-316, 340-349, 379, 380, and 389-391, or a sequence at least 70%, at least 80%, at least 90%, at least 95%, at least 98%, or at least 99% identical thereto.

In some examples of the present disclosure, the heterologous promoter is a promoter of HORST gene. In some examples, the heterologous promoter is operably linked to the isolated nucleic acid molecule encoding a MYB41 amino acid sequence selected from the group including SEQ ID NOs: 38, 40, 44, 46, 49, 52, 70, 76, 77, 78, and 86. In some examples, the heterologous promoter includes an isolated nucleic acid sequence set forth in any one of 2, 236, 254-258, 288, 289, 319-321, 352-354, 383, 394, and 395, or a sequence at least 70%, at least 80%, at least 90%, at least 95%, at least 98%, or at least 99% identical thereto.

In some examples of the present disclosure, the heterologous promoter is a promoter of ASFT gene. In some examples, the heterologous promoter is operably linked to the isolated nucleic acid molecule encoding a MYB41 amino acid sequence selected from the group including SEQ ID NOs: 38, 40, 44, 46, 49, 52, 70, 76, 77, 78, and 86. In some examples, the heterologous promoter includes an isolated nucleic acid sequence set forth in any one of 3, 233, 239-244, 280-285, 314-316, 340-349, 378, 379, and 389-391, or a sequence at least 70%, at least 80%, at least 90%, at least 95%, at least 98%, or at least 99% identical thereto.

In some examples of the present disclosure, the heterologous promoter is a promoter of GPAT5 gene. In some examples, the heterologous promoter is operably linked to the isolated nucleic acid molecule encoding a MYB41 amino acid sequence selected from the group including SEQ ID NOs: 38, 40, 44, 46, 49, 52, 70, 76, 77, 78, and 86. In some examples, the heterologous promoter includes an isolated nucleic acid sequence set forth in any one of 4, 235, 247-253, 286, 287, 317, 318, 350, 351, 381, 382, 392, and 393, or a sequence at least 70%, at least 80%, at least 90%, at least 95%, at least 98%, or at least 99% identical thereto.

In some examples of the present disclosure, the heterologous promoter is a promoter of RALPH gene. In some examples, the heterologous promoter is operably linked to the isolated nucleic acid molecule encoding a MYB41 amino acid sequence selected from the group including SEQ ID NOs: 38, 40, 44, 46, 49, 52, 70, 76, 77, 78, and 86. In some examples, the heterologous promoter includes an isolated nucleic acid sequence set forth in any one of 5, 238, 387, and 388, or a sequence at least 70%, at least 80%, at least 90%, at least 95%, at least 98%, or at least 99% identical thereto.

In some examples of the present disclosure, the heterologous promoter is a promoter of MYB84 gene. In some examples, the heterologous promoter is operably linked to the isolated nucleic acid molecule encoding a MYB41 amino acid sequence selected from the group including SEQ ID NOs: 38, 40, 44, 46, 49, 52, 70, 76, 77, 78, and 86. In some examples, the heterologous promoter includes an isolated nucleic acid sequence set forth in any one of 6, 237, 259-279, 290-313, 322-339, 355-377, 384-386, and 396-411, or a sequence at least 70%, at least 80%, at least 90%, at least 95%, at least 98%, or at least 99% identical thereto.

Additional examples of the present disclosure will be readily ascertained by one skilled in the art of molecular genetics, plant breeding, plant husbandry, agricultural production, and other plant-related technologies upon reading the present application.

### BRIEF DESCRIPTION OF THE DRAWINGS

FIGS. 1A-1G illustrate aspects of Examples 1 and 3 with the promoter HORST (proHORST). FIG. 1A provides a schematic representation of promoter HORST construct. FIG. 1B shows GUS activity assay in Col-0 and one homozygous (MR13) and two T2 Arabidopsis lines (MR270 and MR276) of proHORST. Plants were grown under ½ MS media for 14 days. The yellow arrow shows GUS expression in periderm (secondary growth) and endodermis cells. FIG. 1C provides a schematic representation of the Arabidopsis primary root longitudinal and cross-section. Arabidopsis root cross-section in the differentiated zone has a simple structure composed of the stele (pericycle and vasculature) surrounded by eight one-cell layer endodermis, and one-cell layer cortex and epidermis. Arabidopsis root cross-section undergoing secondary growth in mature root composed of the vasculature (phloem and xylem) and periderm cell layer. FIG. 1D provides a graphical summary of GUS result. The dark and/or pale gray color shows where the GUS activities were detected. FIG. 1E provides a schematic representation of proHORST::MYB41 construct. The MYB41 DNA used in the experiments is the full length genomic DNA, including introns and exons. FIG. 1F provides a representative images of Col-0 and homozygous lines of proHORST::MYB41 grown under ½ MS media for 14 days. FIG. 1G shows Nile Staining showing the first (white arrow) layer of periderm is brighter in proHORST::MYB41 homozygous lines than a Col-0 control imaged at the same stage and exposure.

FIGS. 2A-2H illustrate aspects of Examples 1 and 2 with the promoter FACT (proFACT). FIG. 2A provides a schematic representation of the promoter FACT construct. FIG. 2B shows GUS activity assay in Col-0 and 3 different T2 Arabidopsis lines of proFACT. Plants were grown under ½ MS media for 14 days. The yellow arrow shows GUS expression in periderm (secondary growth) and endodermis cells. FIG. 2C provides a schematic representation of the Arabidopsis primary root longitudinal and cross-section. Arabidopsis root cross-section in the differentiated zone has a simple structure composed of the stele (pericycle and vasculature) surrounded by eight one-cell layer endodermis, and one-cell layer cortex and epidermis. Arabidopsis root cross-section undergoing secondary growth in mature root composed of the vasculature (phloem and xylem) and periderm cell layer. FIG. 2D provides a graphical summary of GUS result. The dark and/or pale gray color shows where the GUS activities were detected. FIG. 2E provides a schematic representation of proFACT::MYB41 construct. The MYB41 DNA used in the experiments is the full length genomic DNA, including introns and exons. FIG. 2F shows expression level of MYB41 in proFACT::MYB41 against two controls; (i) positive control proB-est::MYB41 with β-estradiol treated and (ii) negative control proB-est::MYB41 without β-estradiol treated (Mock). FIG. 2G provides representative images of Col-0 and homozygous lines of proFACT::MYB41 on ½ MS media for 14 days. FIG. 2H Nile Staining showing the first (white arrow) and second layer of periderm (gray arrow) in different proFACT::MYB41 homozygous lines while a Col-0 control imaged at the same stage and exposure only has the first layer of periderm.

FIGS. 3A-3E illustrate quantification of suberin biomarkers by reactive pyrolysis-gas chromatography-mass spectrometry (pyGCMS). FIG. 3A and FIG. 3D provide bar plots showing the levels of suberin biomarkers from dried root tissue from either wild-type, 14 day old Arabidopsis lines or lines expressing either proFACT::MYB41 or proHORST::MYB41 constructs, respectively. Each bar shows the average of three replicate samples where the individual values are represented by the indicated shapes and the error bars represent the standard error of the mean (SEM). In FIG. 3A, data from three independent lines, each harboring the proFACT::MYB41 construct, are represented by the three different shades of gray. In FIG. 3D, data from two independent lines, each harboring the proHORST::MYB41 construct, are shown as indicated by the two shades of dark gray. In this case, three sibling lines (distinguished using symbols) were used for each line. FIG. 3C provides the analysis of the same lines shown in FIG. 3A, but using dried shoot tissue. FIG. 3B and FIG. 3E provide the analysis of the same lines shown in FIG. 3A and FIG. 3D using tissue from 28-day old roots. In all panels, the black arrows denote biomarkers consistently increased compared to the wild-type (WT) control samples.

FIG. 4 illustrates phylogenic tree of different R2R3-MYB subgroups (S11, S9, S24, S10, S10, and S1), which involve with suberin synthesis and suberization.

FIG. 5 shows the impact of proHORST::MYB41 on pennycress plant growth. The data represent T2 progenies of three independent transgenic plants (Lines A, B, and C) and their sibling controls. The number of plants used in the experiment is indicated by the dots on the box plot (Line A transformant n=9; Line A sibling control n=4; Line B transformant n=11; Line B sibling control n=2; Line C transformant n=4; Line C sibling control n=3; Parent line n=15). There was no significant difference between transformants and their sibling controls or transformants and the parent lines (ANOVA and post hoc Tukey test; * P<0.05). All P values were >0.45.

FIGS. 6A-6B quantifies suberin monomer content detected from mature lateral roots of parent line, sibling controls, and proHORST::MYB41 lines. (FIG. 6A) Comparison of suberin monomer content in Line B with its sibling control and parent line. (FIG. 6B) Comparison of suberin monomer content in Line A with its sibling control and parent line. Roots from three independent plants were used as replicates, and the significance was assessed using a two-way ANOVA with multiple comparisons and a post hoc Tukey test. Significance for individual fatty acid monomers was indicated only if at least one of the comparisons yielded a significant difference with p<0.029.

FIG. 7 depicts qPCR data showing expression levels of AtMYB41 in pennycress roots. Expression levels were normalized to the pennycress orthologue of the housekeeping gene IPP2. Expression levels from two individual non-transgenic sibling plants from line A and two non-transgenic sibling plants from line B are shown as negative controls. Expression levels from four individual homozygous proHORST::MYB41 plants from line A and four plants from line B are also shown. Error bars represent the s.e.m of 2 technical replicates.

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand.

In the accompanying sequence listing:
SEQ ID NOs: 1-6 are exemplary promoter sequences that can drive MYB41 expression, as disclosed herein.
SEQ ID NOs: 7-12 are exemplary transformation constructions that can drive MYB41 expression, as disclosed herein.
SEQ ID NO: 13 is an exemplary nucleotide sequence for an Arabidopsis MYB41 gene.
SEQ ID NO: 14 is an exemplary amino acid sequence encoded by an Arabidopsis MYB41 gene.
SEQ ID NO: 15 is an exemplary cDNA encoding an AtMYB41 protein
SEQ ID NOs: 16-21 are exemplary expression cassettes that can be drive MYB41 expression.
SEQ ID NOs: 22-36: are exemplary amino acid sequences for Arabidopsis MYB transcription factors that are disclosed herein to have a regulatory effect on suberin biosynthesis and suberization.
SEQ ID NOs: 37-91 are exemplary amnio acid sequences for orthologs of Arabidopsis MYB41 in other plant species.
SEQ ID NOs: 92-146 are exemplary coding sequences (CDS) for orthologs of Arabidopsis MYB41 in other plant species.
SEQ ID NOs: 147-201 are exemplary nucleotide sequences, including the 5′ and 3′ UTRs, for orthologs of Arabidopsis MYB41 in other plant species.
SEQ ID NOs: 202-216 are exemplary CDS for Arabidopsis MYB transcription factors that are disclosed herein to have a regulatory effect on suberin biosynthesis and suberization.
SEQ ID NOs: 217-232 are exemplary nucleotide sequences, including the 5′ and 3′ UTRs, for Arabidopsis MYB transcription factors that are disclosed herein to have a regulatory effect on suberin biosynthesis and suberization.
SEQ ID NOs: 233-411 are exemplary promoter sequences that can drive MYB41 expression, as disclosed herein.

### DETAILED DESCRIPTION OF THE DISCLOSURE

The present disclosure provides a solution for fighting climate change by utilizing improved plants that remove excess carbon from the earth's atmosphere, sequester it in a soil for a long period, and store it in the plant's biomass. The present disclosure provides methods of identifying genetic materials that can drive increased carbon sequestration in plant cells, plant tissues, plant parts and whole plants, wherein the carbon is stored in suberin deposited in the plants. Also, the present disclosure provides methods of transferring genetic materials to plants in order to give rise to traits that increase suberin content of plant cells, plant tissues, plant parts and whole plants. Furthermore, the present disclosure provides newly-identified genetic components and methods of generating genetically-modified or gene-edited plants, plant cells, tissues, and seeds, having modified carbon sequestration or increased suberin content. The disclosure further provides methods of breeding with said genetically engineered plants to transfer the increased suberin trait to other lines and plant species.

### I. Terms

Unless stated otherwise, all technical and scientific terms used herein have the same meaning as commonly understood by those of ordinary skill in the art to which the disclosure belongs. While the following terms are believed to be well understood by one of ordinary skill in the art, the following explanations are set forth to facilitate explanation of the presently disclosed subject matter. Although any methods and materials similar or equivalent to those described herein can be used in the practice or testing of the present disclosure, preferred methods and materials are described. The following terms are explained below. These explanations are for illustrative purposes and are not intended to limit the common meaning in the art of the terms.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain examples, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some examples, it is 20 nucleotides. A portion of a polypeptide useful as an epitope may be as short as 4 amino acids. A portion of a polypeptide that performs the function of the full-length polypeptide would generally be longer than 4 amino acids. In some examples, a fragment of a polypeptide or polynucleotide comprises at least 10%, 20%, 30%, 40%, 50%, 60%, 70%, 80%, 90%, 95%, 96%, 97%, 98%, or 99% of the entire length of the reference polypeptide or polynucleotide. In some examples, a polypeptide or polynucleotide fragment may contain 5, 10, 15, 20, 25, 30, 35, 40, 45, 50, 60, 70, 80, 90, 100, 200, 300, 400, 500, 600, 700, 800, 900, 1000, 2000 or more nucleotides or amino acids.

As used herein, the term “codon optimization” implies that the codon usage of a DNA or RNA is adapted to that of a cell or organism of interest to improve the transcription rate of said recombinant nucleic acid in the cell or organism of interest. A target nucleic acid can be modified at one position due to the codon degeneracy, whereas this modification will still lead to the same amino acid sequence at that position after translation, which is achieved by codon optimization to take into consideration the species-specific codon usage of a target cell or organism.

As used herein, the term “endogenous” or “endogenous gene,” refers to the naturally occurring gene, in the location in which it is naturally found within the host cell genome. “Endogenous gene” is synonymous with “native gene” as used herein. An endogenous gene as described herein can include alleles of naturally occurring genes that have been mutated according to any of the methods of the present disclosure, i.e. an endogenous gene could have been modified at some point by traditional plant breeding methods and/or next generation plant breeding methods.

As used herein, the term “exogenous” refers to a substance coming from some source other than its native source. For example, the terms “exogenous protein,” or “exogenous gene” refer to a protein or gene from a non-native source, and that has been artificially supplied to a biological system. As used herein, the term “exogenous” is used interchangeably with the term “heterologous,” and refers to a substance coming from some source other than its native source.

The terms “genetically engineered host cell,” “recombinant host cell,” and “recombinant strain” are used interchangeably herein and refer to host cells that have been genetically engineered by the methods of the present disclosure. Thus, the terms include a host cell (e.g., bacteria, yeast cell, fungal cell, CHO, human cell, plant cell, protoplast derived from plant, callus, etc.) that has been genetically altered, modified, or engineered, such that it exhibits an altered, modified, or different genotype and/or phenotype (e.g., when the genetic modification affects coding nucleic acid sequences), as compared to the naturally-occurring host cell from which it was derived. It is understood that the terms refer not only to the particular recombinant host cell in question, but also to the progeny or potential progeny of such a host cell.

As used herein, the term “heterologous” refers to a substance coming from some source or location other than its native source or location. In some examples, the term “heterologous nucleic acid” refers to a nucleic acid sequence that is not naturally found in the particular organism. For example, the term “heterologous promoter” may refer to a promoter that has been taken from one source organism and utilized in another organism, in which the promoter is not naturally found. However, the term “heterologous promoter” may also refer to a promoter that is from within the same source organism, but has merely been moved to a novel location, in which said promoter is not normally located.

Heterologous gene sequences can be introduced into a target cell by using an “expression vector,” which can be a eukaryotic expression vector, for example a plant expression vector. Methods used to construct vectors are described in various publications. In particular, techniques for constructing suitable vectors, including a description of the functional components such as promoters, enhancers, termination and polyadenylation signals, selection markers, origins of replication, and splicing signals, are reviewed in the prior art. Vectors may include but are not limited to plasmid vectors, phagemids, cosmids, artificial/mini-chromosomes (e.g. ACE), or viral vectors such as baculovirus, retrovirus, adenovirus, adeno-associated virus, herpes simplex virus, retroviruses, bacteriophages. The eukaryotic expression vectors will typically contain also prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known in the art and some are commercially available from companies such as Stratagene, La Jolla, Calif.; Invitrogen, Carlsbad, Calif.; Promega, Madison, Wis. or BD Biosciences Clontech, Palo Alto, Calif. In one example the expression vector comprises at least one nucleic acid sequence which is a regulatory sequence necessary for transcription and translation of nucleotide sequences that encode for a peptide/polypeptide/protein of interest.

As used herein, the term “naturally occurring” as applied to a nucleic acid, a polypeptide, a cell, or an organism, refers to a nucleic acid, polypeptide, cell, or organism that is found in nature. The term “naturally occurring” may refer to a gene or sequence derived from a naturally occurring source. Thus, for the purposes of this disclosure, a “non-naturally occurring” sequence is a sequence that has been synthesized, mutated, engineered, edited, or otherwise modified to have a different sequence from known natural sequences. In some examples, the modification may be at the protein level (e.g., amino acid substitutions). In other examples, the modification may be at the DNA level (e.g., nucleotide substitutions).

As used herein, the term “nucleotide change” or “nucleotide modification” refers to, e.g., nucleotide substitution, deletion, and/or insertion, as is well understood in the art. For example, such nucleotide changes/modifications include mutations containing alterations that produce silent substitutions, additions, or deletions, but do not alter the properties or activities of the encoded protein or how the proteins are made. As another example, such nucleotide changes/modifications include mutations containing alterations that produce replacement substitutions, additions, or deletions, that alter the properties or activities of the encoded protein or how the proteins are made.

As used herein, the term “protein modification” refers to, e.g., amino acid substitution, amino acid modification, deletion, and/or insertion, as is well understood in the art.

The term “next generation plant breeding” refers to a host of plant breeding tools and methodologies that are available to today's breeder. One distinguishing feature of next generation plant breeding is that the breeder is no longer confined to relying upon observed phenotypic variation, in order to infer underlying genetic causes for a given trait. Rather, next generation plant breeding may include the utilization of molecular markers and marker assisted selection (MAS), such that the breeder can directly observe movement of alleles and genetic elements of interest from one plant in the breeding population to another, and is not confined to merely observing phenotype. Further, next generation plant breeding methods are not confined to utilizing natural genetic variation found within a plant population. Rather, the breeder utilizing next generation plant breeding methodology can access a host of modern genetic engineering tools that directly alter/change/edit the plant's underlying genetic architecture in a targeted manner, in order to bring about a phenotypic trait of interest. In aspects, the plants bred with a next generation plant breeding methodology are indistinguishable from a plant that was bred in a traditional manner, as the resulting end product plant could theoretically be developed by either method. In particular aspects, a next generation plant breeding methodology may result in a plant that comprises: a genetic modification that is a deletion or insertion of any size; a genetic modification that is one or more base pair substitution; a genetic modification that is an introduction of nucleic acid sequences from within the plant's natural gene pool (e.g. any plant that could be crossed or bred with a plant of interest) or from editing of nucleic acid sequences in a plant to correspond to a sequence known to occur in the plant's natural gene pool; and offspring of said plants.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

The terms “polynucleotide,” “nucleic acid,” and “nucleotide sequence,” used interchangeably herein, refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. This term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. It also includes modified nucleic acids such as methylated and/or capped nucleic acids, nucleic acids containing modified bases, backbone modifications, and the like. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” “nucleic acid,” and “nucleotide sequence” should be understood to include, as applicable to the examples being described, single-stranded (such as sense or antisense) and double-stranded polynucleotides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used, then the choice of vector is dependent upon the method that will be used to transform host cells. For example, a plasmid vector can be used. The skilled artisan is well aware of the genetic elements that must be present on the vector in order to successfully transform, select and propagate host cells comprising any of the isolated nucleic acid fragments of the disclosure. Different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

The term “traditional plant breeding” refers to the utilization of natural variation found within a plant population as a source for alleles and genetic variants that impart a trait of interest to a given plant. Traditional breeding methods make use of crossing procedures that rely largely upon observed phenotypic variation to infer causative allele association. That is, traditional plant breeding relies upon observations of expressed phenotype of a given plant to infer underlying genetic cause. These observations are utilized to inform the breeding procedure in order to move allelic variation into germplasm of interest. Further, traditional plant breeding has also been characterized as comprising random mutagenesis techniques, which can be used to introduce genetic variation into a given germplasm. These random mutagenesis techniques may include chemical and/or radiation-based mutagenesis procedures. Consequently, one key feature of traditional plant breeding, is that the breeder does not utilize a genetic engineering tool that directly alters/changes/edits the plant's underlying genetic architecture in a targeted manner, in order to introduce genetic diversity and bring about a phenotypic trait of interest.

A “CRISPR-associated effector” as used herein can thus include any nuclease, nickase, or recombinase associated with the CRISPR (Clustered Regularly Interspaced Short Palindromic Repeats), having the capacity to introduce a single- or double-strand cleavage into a genomic target site, or having the capacity to introduce a targeted modification, including a point mutation, an insertion, or a deletion, into a genomic target site of interest. At least one CRISPR-associated effector can act on its own, or in combination with other molecules as part of a molecular complex. The CRISPR-associated effector can be present as fusion molecule, or as individual molecules associating by or being associated by at least one of a covalent or non-covalent interaction with gRNA and/or target site so that the components of the CRISPR-associated complex are brought into close physical proximity.

The term “Cas9 nuclease” and “Cas9” can be used interchangeably herein, which refer to a RNA-guided DNA endonuclease enzyme associated with the CRISPR (Clustered Regularly Interspaced Short Palindromic Repeats), including the Cas9 protein or fragments thereof (such as a protein comprising an active DNA cleavage domain of Cas9 and/or a gRNA binding domain of Cas9). Cas9 is a component of the CRISPR/Cas genome editing system, which targets and cleaves a DNA target sequence to form a DNA double strand breaks (DSB) under the guidance of a guide RNA.

The term “CRISPR RNA” or “crRNA” refers to the RNA strand responsible for hybridizing with target DNA sequences, and recruiting CRISPR endonucleases and/or CRISPR-associated effectors. crRNAs may be naturally occurring, or may be synthesized according to any known method of producing RNA.

The term “tracrRNA” refers to a small trans-encoded RNA. TracrRNA is complementary to and base pairs with crRNA to form a crRNA/tracrRNA hybrid, capable of recruiting CRISPR endonucleases and/or CRISPR-associated effectors to target sequences.

The term “Guide RNA” or “gRNA” as used herein refers to an RNA sequence or combination of sequences capable of recruiting a CRISPR endonuclease and/or CRISPR-associated effectors to a target sequence. Typically gRNA is composed of crRNA and tracrRNA molecules forming complexes through partial complement, wherein crRNA comprises a sequence that is sufficiently complementary to a target sequence for hybridization and directs the CRISPR complex (i.e. Cas9-crRNA/tracrRNA hybrid) to specifically bind to the target sequence. Also, single guide RNA (sgRNA) can be designed, which comprises the characteristics of both crRNA and tracrRNA. Therefore, as used herein, a guide RNA can be a natural or synthetic crRNA (e.g., for Cpf1), a natural or synthetic crRNA/tracrRNA hybrid (e.g., for Cas9), or a single-guide RNA (sgRNA).

The term “guide sequence” or “spacer sequence” refers to the portion of a crRNA or guide RNA (gRNA) that is responsible for hybridizing with the target DNA.

The term “protospacer” refers to the DNA sequence targeted by a guide sequence of crRNA or gRNA. In some examples, the protospacer sequence hybridizes with the crRNA or gRNA guide (spacer) sequence of a CRISPR complex.

The term “CRISPR landing site” as used herein, refers to a DNA sequence capable of being targeted by a CRISPR-Cas complex. In some examples, a CRISPR landing site comprises a proximately placed protospacer/Protopacer Adjacent Motif combination sequence that is capable of being cleaved by a CRISPR complex.

The term “CRISPR complex”, “CRISPR endonuclease complex”, “CRISPR Cas complex”, or “CRISPR-gRNA complex” are used interchangeably herein. “CRISPR complex” refers to a Cas9 nuclease and/or a CRISPR-associated effectors complexed with a guide RNA (gRNA). The term “CRISPR complex” thus refers to a combination of CRISPR endonuclease and guide RNA capable of inducing a double stranded break at a CRISPR landing site. In some examples, “CRISPR complex” of the present disclosure refers to a combination of catalytically dead Cas9 protein and guide RNA capable of targeting a target sequence, but not capable of inducing a double stranded break at a CRISPR landing site because it loses a nuclease activity. In other examples, “CRISPR complex” of the present disclosure refers to a combination of Cas9 nickase and guide RNA capable of introducing gRNA-targeted single-strand breaks in DNA instead of the double-strand breaks created by wild type Cas enzymes.

As used herein, the term “directing sequence-specific binding” in the context of CRISPR complexes refers to a guide RNA's ability to recruit a CRISPR endonuclease and/or a CRISPR-associated effectors to a CRISPR landing site.

As used herein the term “targeted” refers to the expectation that one item or molecule will interact with another item or molecule with a degree of specificity, so as to exclude non-targeted items or molecules. For example, a first polynucleotide that is targeted to a second polynucleotide, according to the present disclosure has been designed to hybridize with the second polynucleotide in a sequence specific manner (e.g., via Watson-Crick base pairing). In some examples, the selected region of hybridization is designed so as to render the hybridization unique to the one, or more targeted regions. A second polynucleotide can cease to be a target of a first targeting polynucleotide, if its targeting sequence (region of hybridization) is mutated, or is otherwise removed/separated from the second polynucleotide. Furthermore, “targeted” can be interchangeably used with “site-specific” or “site-directed,” which refers to an action of molecular biology which uses information on the sequence of a genomic region of interest to be modified, and which further relies on information of the mechanism of action of molecular tools, e.g., nucleases, including CRISPR nucleases and variants thereof, TALENs, ZFNs, meganucleases or recombinases, DNA-modifying enzymes, including base modifying enzymes like cytidine deaminase enzymes, histone modifying enzymes and the like, DNA-binding proteins, cr/tracr RNAs, guide RNAs and the like.

The term “seed region” refers to the critical portion of a crRNA's or guide RNA's guide sequence that is most susceptible to mismatches with their targets. In some examples, a single mismatch in the seed region of a crRNA/gRNA can render a CRISPR complex inactive at that binding site. In some examples, the seed regions for Cas9 endonucleases are located along the last ~12 nts of the 3′ portion of the guide sequence, which correspond (hybridize) to the portion of the protospacer target sequence that is adjacent to the PAM. In some examples, the seed regions for Cpf1 endonucleases are located along the first ~5 nts of the 5′ portion of the guide sequence, which correspond (hybridize) to the portion of the protospacer target sequence adjacent to the PAM.

The terms “sequence identity”, “% sequence identity”, “% identity”, “% identical” or “sequence alignment” are used interchangeably herein and refer to the comparison of a first nucleic acid sequence to a second nucleic acid sequence, or a comparison of a first amino acid sequence to a second amino acid sequence and is calculated as a percentage based on the comparison. The result of this calculation can be described as “percent identical” or “percent ID.” A sequence identity may be determined by a program, which produces an alignment, and calculates identity counting both mismatches at a single position and gaps at a single position as non-identical positions in final sequence identity calculation. The sequence identity is determined over the entire length of the first and second nucleic acid sequence. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment can involve scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). The comparison of sequences and determination of percent identity between two sequences can be accomplished using a mathematical algorithm. An example of a local alignment algorithm utilized for the comparison of sequences is the NCBI Basic Local Alignment Search Tool (BLAST®) (Altschul et al. 1990 J. Mol. Biol. 215:403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal omega. Unless otherwise stated, references to sequence identity used herein refer to the NCBI Basic Local Alignment Search Tool (BLAST®).

The term “complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and Santa Lucia, Nucl. Acids Research, 2005; 33 (19): 6258-6267.

As referred to herein, a “complementary nucleic acid sequence” is a nucleic acid sequence comprising a sequence of nucleotides that enables it to non-covalently bind to another nucleic acid in a sequence-specific, antiparallel, manner (i.e., a nucleic acid specifically binds to a complementary nucleic acid) under the appropriate in vitro and/or in vivo conditions of temperature and solution ionic strength.

Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters, and MUSCLE (Multiple Sequence Comparison by Log-Expection; a computer software licensed as public domain).

The term “modified” refers to a substance or compound (e.g., a cell, a polynucleotide sequence, and/or a polypeptide sequence) that has been altered or changed as compared to the corresponding unmodified substance or compound.

“Isolated” refers to a material that is free to varying degrees from components which normally accompany it as found in its native state.

The term “gene edited plant, part or cell” as used herein refers to a plant, part or cell that comprises one or more endogenous genes that are edited by a gene editing system. The gene editing system of the present disclosure comprises a targeting element and/or an editing element. The targeting element is capable of recognizing a target genomic sequence. The editing element is capable of modifying the target genomic sequence, e.g., by substitution or insertion of one or more nucleotides in the genomic sequence, deletion of one or more nucleotides in the genomic sequence, alteration of genomic sequences to include regulatory sequences, insertion of transgenes at a safe harbor genomic site or other specific location in the genome, or any combination thereof. The targeting element and the editing element can be on the same nucleic acid molecule or different nucleic acid molecules.

The term “plant part” includes differentiated and undifferentiated tissues including, but not limited to: plant organs, plant tissues, roots, stems, shoots, rootstocks, scions, stipules, petals, leaves, flowers, ovules, pollens, bracts, petioles, internodes, bark, pubescence, tillers, rhizomes, fronds, blades, stamens, fruits, seeds, tumor tissue and plant cells (e.g., single cells, protoplasts, embryos, and callus tissue). Plant cells include, without limitation, cells from seeds, suspension cultures, embryos, meristematic regions, callus tissue, leaves, roots, shoots, gametophytes, sporophytes, pollen and microspores. The plant tissue may be in a plant or in a plant organ, tissue or cell culture.

The terms “transgene” or “transgenic” as used herein refer to at least one nucleic acid sequence that is taken from the genome of one organism, or produced synthetically, and which is then introduced into a host cell or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation and thus inheritance of the respective at least one molecule introduced into the genome of a cell. The terms “transgene-free” refers to a condition that transgene is not present or found in the genome of a host cell or tissue or organism of interest.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

By “biologically active portion” is meant a portion of a full-length parent peptide or polypeptide which portion retains an activity of the parent molecule. For example, a biologically active portion of polypeptide of the disclosure will retain the ability to increase and/or enhance suberin levels in plant cells, tissues and whole plants. As used herein, the term “biologically active portion” includes deletion mutants and peptides, for example of at least about 8, 9, 10, 11, 12, 13, 14, 15, 16, 17, 18, 19, 20, 21, 22, 23, 24, 25, 30, 40, 50, 60, 70, 80, 90, 100, 120, 150, 300, 400, 500, 600, 700, 800, 900 or 1000 contiguous amino acids, which comprise an activity of a parent molecule. Portions of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a peptide or polypeptide of the disclosure with proteinases such as endoLys-C, endoArg-C, endoGlu-C and staphylococcus V8-protease. The digested fragments can be purified by, for example, high performance liquid chromatographic (HPLC) techniques. Recombinant nucleic acid techniques can also be used to produce such portions.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

The terms “growing” or “regeneration” as used herein mean growing a whole, differentiated plant from a plant cell, a group of plant cells, a plant part (including seeds), or a plant piece (e.g., from a protoplast, callus, or tissue part).

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as described elsewhere herein.

By “obtained from” is meant that a sample such as, for example, a nucleic acid extract or polypeptide extract is isolated from, or derived from, a particular source. For example, the extract may be isolated directly from plants.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present disclosure are biologically active, that is they continue to possess the desired biological activity of the native protein, that is, modulating or regulatory activity as described herein. Such variants may result from, for example, genetic polymorphism or from human manipulation. Biologically active variants of a native R protein of the disclosure will have at least 40%, 50%, 60%, 70%, generally at least 75%, 80%, 85%, preferably about 90% to 95% or more, and more preferably about 98% or more sequence identity to the amino acid sequence for the native protein as determined by sequence alignment programs described elsewhere herein using default parameters. A biologically active variant of a protein of the disclosure may differ from that protein by as few as 1-15 amino acid residues, as few as 1-10, such as 6-10, as few as 5, as few as 4, 3, 2, or even 1 amino acid residue.

The proteins of the disclosure may be altered in various ways including amino acid substitutions, deletions, truncations, and insertions. Methods for such manipulations are generally known in the art. For example, amino acid sequence variants of the R proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another, Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development in animal and/or plant.

As used herein, the term “vector”, “plasmid”, or “construct” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, recombinant plant viruses. Non-limiting examples of plant viruses include, TMV-mediated (transient) transfection into tobacco (Tuipe, T-H et al (1993), J. Virology Meth, 42:227-239), ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae). Detailed classification information of plant viruses can be found in Fauquet et al (2008, “Geminivirus strain demarcation and nomenclature”. Archives of Virology 153:783-821, incorporated herein by reference in its entirety), and Khan et al. (Plant viruses as molecular pathogens; Publisher Routledge, 2002, ISBN 1560228954, 9781560228950). Examples of non-viral vectors include, but are not limited to, liposomes, polyamine derivatives of DNA, and the like.

Also, “vector” includes, inter alia, any plasmid, cosmid, phage or Agrobacterium binary vector in double or single stranded linear or circular form which may or may not be self-transmissible or mobilizable, and which can transform prokaryotic or eukaryotic host either by integration into the cellular genome or exist extrachromosomally (e.g. autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, e.g. bacterial, or plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The term “plant” includes reference to whole plants, or plants having more than one type of tissue or organ.

As used herein, the term “molecular marker” or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Techniques for mapping of molecular markers in the vicinity of an allele are for instance described in Lefebvre and Chevre, 1995; Lorez and Wenzel, 2007, Srivastava and Narula, 2004, Meksem and Kahl, 2005, Phillips and Vasil, 2001. General information concerning AFLP technology can be found in Vos et al. (1995, AFLP: a new technique for DNA fingerprinting, Nucleic Acids Res. 1995 Nov. 11; 23 (21): 4407-4414).

As used herein, the term “homologous” or “homolog” is known in the art and refers to related sequences that share a common ancestor or family member and are determined based on the degree of sequence identity. The terms “homology”, “homologous”, “substantially similar” and “corresponding substantially” are used interchangeably herein. Homologs usually control, mediate, or influence the same or similar biochemical pathways, yet particular homologs may give rise to differing phenotypes. It is therefore understood that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared.

The term “homolog” is sometimes used to apply to the relationship between genes separated by the event of speciation (see “ortholog”) or to the relationship between genes separated by the event of genetic duplication (see “paralog”).

The term “homeolog” refers to a homologous gene or chromosome, resulting from polyploidy or chromosomal duplication events. This contrasts with the more common ‘homolog’, which is described immediately above.

The term “ortholog” refers to genes in different species that evolved from a common ancestral gene by speciation. Normally, orthologs retain the same function in the course of evolution. Identification of orthologs is critical for reliable prediction of gene function in newly sequenced genomes.

The term “paralog” refers to genes related by duplication within a genome. While orthologs generally retain the same function in the course of evolution, paralogs can evolve new functions, even if these are related to the original one.

“Homologous sequences” or “homologs” or “orthologs” are thought, believed, or known to be functionally related. A functional relationship may be indicated in any one of a number of ways, including, but not limited to: (a) degree of sequence identity and/or (b) the same or similar biological function. Homology can be determined using software programs readily available in the art, such as those discussed in Current Protocols in Molecular Biology (F. M. Ausubel et al., eds., 1987) Supplement 30, section 7.718, Table 7.71. Some alignment programs are Mac Vector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA).

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the terms “wildtype check”, “wildtype” or “check” all refer to a first cell, tissue culture, part or organism which is essentially genetically the same as a second cell, tissue culture, part or organism, respectively, except that the corresponding second cell, tissue culture, part or organism comprises a heterologous genetic element not present in the first cell, tissue culture, part or organism. Thus, for example, a first plant would be a wildtype check relative to a second plant where the only meaningful genetic difference between the two is that the second plant comprises a heterologous gene (e.g., MYB41) not present in the first plant.

As used herein, the term “variety” or “cultivar” means a group of similar plants that by structural features and performance can be identified from other varieties within the same species. The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

A variety is deemed to be essentially derived from another variety (‘the initial variety’) when: (i) it is predominantly derived from the initial variety, or from a variety that is itself predominantly derived from the initial variety, while retaining the expression of the essential characteristics that result from the genotype or combination of genotypes of the initial variety; (ii) it is clearly distinguishable from the initial variety; and, (iii) except for the differences which result from the act of derivation, it conforms to the initial variety in the expression of the essential characteristics that result from the genotype or combination of genotypes of the initial variety. UPOV, Article 14 (5) (b).

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as described elsewhere herein.

A probe comprises an identifiable, isolated nucleic acid that recognizes a target nucleic acid sequence. A probe includes a nucleic acid that is attached to an addressable location, a detectable label or other reporter molecule and that hybridizes to a target sequence. Typical labels include radioactive isotopes, enzyme substrates, co-factors, ligands, chemiluminescent or fluorescent agents, haptens, and enzymes. Methods for labelling and guidance in the choice of labels appropriate for various purposes are discussed, for example, in Sambrook et al. (ed.), Molecular Cloning: A Laboratory Manual, 2nd ed., vol. 1-3, Cold Spring Harbor Laboratory Press, Cold Spring Harbor, NY, 1989 and Ausubel et al. Short Protocols in Molecular Biology, 4th ed., John Wiley & Sons, Inc., 1999.

Methods for preparing and using nucleic acid probes and primers are described, for example, in Sambrook et al. (ed.), Molecular Cloning: A Laboratory Manual, 2nd ed., vol. 1-3, Cold Spring Harbor Laboratory Press, Cold Spring Harbor, NY, 1989; Ausubel et al. Short Protocols in Molecular Biology, 4th ed., John Wiley & Sons, Inc., 1999; and Innis et al. PCR Protocols, A Guide to Methods and Applications, Academic Press, Inc., San Diego, CA, 1990. Amplification primer pairs can be derived from a known sequence, for example, by using computer programs intended for that purpose such as PRIMER (Version 0.5, 1991, Whitehead Institute for Biomedical Research, Cambridge, MA). The specificity of a particular probe or primer increases with its length. Thus, in order to obtain greater specificity, probes and primers can be selected that comprise at least 20, 25, 30, 35, 40, 45, 50 or more consecutive nucleotides of a target nucleotide sequences.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3rd ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

### II. Suberin

As used herein, “suberin” refers to a highly hydrophobic and a somewhat ‘rubbery’ material. In roots, suberin is deposited in the radial and transverse cell walls of the endodermal cells. This structure, known as the Casparian strip or Casparian band, functions to prevent water and nutrients taken up by the root from entering the stele through the apoplast. Instead, water must bypass the endodermis via the symplast. This allows the plant to select the solutes that pass further into the plant. It thus forms an important barrier to harmful solutes. For example, mangroves use suberin to minimize salt intake from their littoral habitat.

Suberin is found in the phellem layer of the periderm. This is outermost layer of the bark. The cells in this layer are dead and abundant in suberin, preventing water loss from the tissues below. Suberin is also a major component of cork. Suberin can also be found in various other plant structures. For example, they are present in the lenticels on the stems of many plants and the net structure in the rind of a netted melon is composed of suberized cells, and in the roots.

When found in plant roots, it absorbs carbon yet resists decomposition that releases carbon back into the atmosphere, enriches soil and helps plants resist stress.

Thus, plants having increased root mass and suberin content are able to store more carbon.

### III. MYB Transcription Factor Family

The MYB family of proteins is ubiquitous in eukaryotes and exhibits functional diversity. These proteins mostly act as transcription factors and their DNA-binding ability varies according to the number of MYB domain repeats. In plants, the MYB family has significantly expanded, especially the R2R3-MYB family. The members of this family perform plant-specific functions, as seen from their comprehensive functional characterization in Arabidopsis (Arabidopsis thaliana). MYB proteins play a crucial role in regulating the development, metabolism, and response to biotic and abiotic stresses. MYB transcription factors in Arabidopsis are described in detail in Dubos et al. (2010) Trends Plant Sci. 2010 October; 15 (10): 573-81, which is incorporated by reference herein.

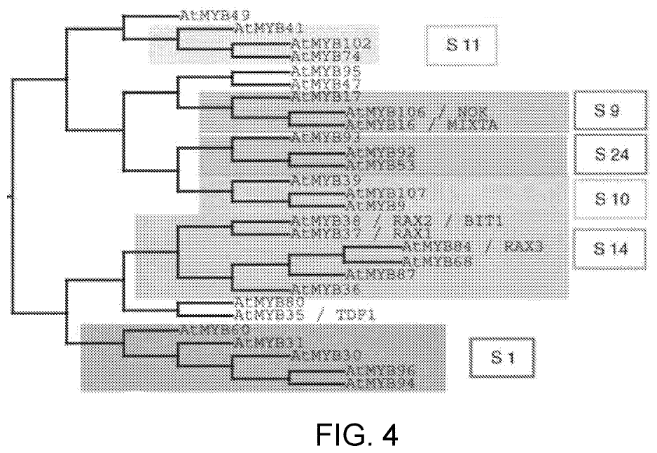

Most plant MYB genes produce R2R3-MYB proteins that are believed to have developed from an ancestor R1R2R3-MYB gene. R2R3-MYB transcription factors have a modular structure with a MYB domain at the N-terminal DNA-binding domain and an activation or repression domain usually found at the C-terminal. While the MYB domain is highly conserved, the other regions of R2R3-MYB proteins are variable. R2R3-MYB proteins are divided into subgroups based on the conservation of the DNA-binding domain and amino acid motifs in the C-terminal domains. FIG. 4 shows a phylogenic tree of the different R2R3-MYB subgroups (S11, S9, S24, S10, S10, and S1) that relate to suberin synthesis and suberization. AtMYB41 belongs to subgroup 11 as presented in FIG. 4.

Table 1 presents 16 Arabidopsis MYB transcription factors that can have a regulatory effect on suberin biosynthesis and suberization.

TABLE 1

| | | Relevance to Suberin Pathway and/or | SEQ ID NO | SEQ ID NO | SEQ ID NO |
|---|---|---|---|---|---|
| Locus ID | Symbol | Suberization | (Protein) | (CDS) | (Gene*) |
| AT1G34670 | MYB93 | CASP1::MYB93 increased casparian strip | 22 | 202 | 217 |
| | | suberization; myb53, 41, 92, 93 quad mutant | | | |
| | | precluded endodermal suberization; | | | |
| | | Overexpression led to increased root | | | |
| | | suberization | | | |
| AT1G74080 | MYB122 | Overexpression delayed endodermal strip | 23 | 203 | 218 |
| | | suberization | | | |
| AT3G02940 | MYB107 | Mutant disrupts seed coat suberization; | 24 | 204 | 219 |
| | | Kiwi ortholog expression in N. benthamiana | | | |
| | | leaves increases suberin monomer levels | | | |
| AT3G12720 | MYB67 | scRNA-seq putative periderm cells cluster | 25 | 205 | 220 |
| | | “bridge” | | | |
| AT3G49690 | MYB84 | Overexpression led to increased root | 26 | 206 | 221 |
| | | suberization | | | |
| AT4G05100 | MYB74 | Potato ortholog overexpression in N. | 27 | 207 | 222 |
| | | benthamiana induces formation of suberin | | | |
| | | lamellae; | | | |
| | | Ectopic overexpression led to increased nile | | | |
| | | red staining in 14d old arabidopsis roots; | | | |
| | | Overexpression led to increased root | | | |
| | | suberization | | | |
| AT4G09460 | MYB6 | Overexpression delayed endodermal strip | 28 | 208 | 223 |
| | | suberization | | | |
| AT4G17785 | MYB39 | Overexpression and mutant alter suberin | 29 | 209 | 224 |
| | | lamellae formation and casparian strip | | | |
| | | suberization; | | | |
| | | Overexpression led to increased root | | | |
| | | suberization | | | |
| AT4G21440 | MYB102 | Potato ortholog overexpression in N. | 30 | 210 | 225 |
| | | benthamiana induces formation of suberin | | | |
| | | lamellae; | | | |
| | | Upregulated in poplar cork tissue | | | |
| AT4G28110 | MYB41 | Overexpression led to increased root | 14 | 15 | 232 |
| | | suberization; | | | |
| | | Ectopic overexpression led to increased nile | | | |
| | | red staining in 14d old arabidopsis roots; | | | |
| | | Kiwi ortholog expression in N. benthamiana | | | |
| | | leaves increases suberin monomer levels; | | | |
| | | CASP1::MYB41 increased casparian strip | | | |
| | | suberization; myb53, 41, 92, 93 quad mutant | | | |
| | | precluded endodermal suberization; | | | |
| | | Grape ortholog expression in N. | | | |
| | | benthamiana leaves increases suberin | | | |
| | | monomer levels | | | |
| AT4G38620 | MYB4 | Kiwi ortholog expression in N. benthamiana | 31 | 211 | 226 |
| | | suppressed suberin biosynthetic gene | | | |
| | | expression and reduced suberin monomer- | | | |
| | | like molecules content | | | |
| AT5G10280 | MYB92 | CASP1::MYB92 increased casparian strip | 32 | 212 | 22 |
| | | suberization; myb53, 41, 92, 93 quad mutant | | | |
| | | precluded endodermal suberization; | | | |
| | | Overexpression led to increased root | | | |
| | | suberization | | | |
| AT5G16770 | MYB9 | Overexpression led to increased root | 33 | 213 | 22 |
| | | suberization; | | | |
| | | Mutant disrupts seed coat suberization | | | |
| AT5G57620 | MYB36 | Overexpression led to increased root | 34 | 214 | 229 |
| | | suberization | | | |
| AT5G65230 | MYB53 | CASP1::MYB53 increased casparian strip | 35 | 215 | 230 |
| | | suberization; | | | |
| | | myb53, 41, 92, 93 quad mutant precluded | | | |
| | | endodermal suberization | | | |
| AT5G65790 | MYB68 | Overexpression led to increased root | 36 | 216 | 23 |
| | | suberization; | | | |
| | | Apple ortholog overexpression led to | | | |
| | | increased suberin monomer content in N. | | | |
| | | benthamiana leaves | | | |

*Gene sequence includes 5′ and 3′ UTRs

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence set forth in any one of SEQ ID NOs: 14 and 22-36.

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence encoded by nucleic acid sequences of MYB transcription factors, associated with suberin synthesis or suberization, set forth in any one of SEQ ID NOs: 15 and 202-216.

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence encoded by nucleic acid sequences of MYB41 gene orthologs set forth in any one of SEQ ID NOS: 217-232.

In some examples, the present disclosure provides an isolated nucleic acid molecule comprising a nucleic acid sequence encoding a MYB transcription factor whose amino acid sequence has at least 80% identical to any one of SEQ ID NOs: 14 and 22-36 and/or a nucleic acid sequence having at least 80% identical to any one of SEQ ID NOs: 15 and 202-232, operably linked to a nucleic acid sequence encoding a heterologous promoter, wherein expression of the isolated nucleic acid molecule in a plant results in increased levels of suberin as compared to wild-type check plants lacking the isolated nucleic acid molecule. In some examples, the increased levels of suberin occur by generating additional periderm cells and/or depositing more suberin in existing periderm cells.

In some examples, the amino acid sequence of MYB TFs related to suberin synthesis or suberization is SEQ ID NOs: 14 and 22-36. In some examples, the nucleic acid sequence related to suberin synthesis or suberization is any one of SEQ ID NOs: 15 and 202-232.

In some examples, the MYB transcription factor involved with suberin synthesis or suberization is Arabidopsis MYB93, Arabidopsis MYB122, Arabidopsis MYB107, Arabidopsis MYB67, Arabidopsis MYB84, Arabidopsis MYB74, Arabidopsis MYB6, Arabidopsis MYB39, Arabidopsis MYB102, Arabidopsis MYB41, Arabidopsis MYB4, Arabidopsis MYB92, Arabidopsis MYB9, Arabidopsis MYB36, Arabidopsis MYB53, or Arabidopsis MYB68.

In some examples, the present disclosure provides a genetically engineered plant, that includes an isolated nucleic acid molecule encoding a MYB transcription factor whose amino acid sequence that is selected from the group consisting of SEQ ID NOs: 14 and 22-36, operably linked to a nucleic acid sequence encoding a heterologous promoter, wherein expression of the isolated nucleic acid molecule in a plant results in increased levels of suberin as compared to wild-type check plants lacking the isolated nucleic acid molecule.

In some examples, the present disclosure provides a genetically engineered plant, that includes an isolated nucleic acid molecule that is selected from the group consisting of SEQ ID NOS: 15 and 202-203, operably linked to a nucleic acid sequence encoding a heterologous promoter, wherein expression of the isolated nucleic acid molecule in a plant results in increased levels of suberin as compared to wild-type check plants lacking the isolated nucleic acid molecule.

In some examples, the heterologous promoter is a promoter of FACT gene, which is operably linked to the isolated nucleic acid molecule encoding a MYB transcription factor whose amino acid sequence that is selected from the group consisting of SEQ ID NOs: 14 and 22-36. In some examples, the heterologous promoter is a promoter of HORST gene, which is operably linked to the isolated nucleic acid molecule encoding a MYB transcription factor whose amino acid sequence that is selected from the group consisting of SEQ ID NOs: 14 and 22-36. In some examples, the heterologous promoter is a promoter of ASFT gene, which is operably linked to the isolated nucleic acid molecule encoding a MYB transcription factor whose amino acid sequence that is selected from the group consisting of SEQ ID NOs: 14 and 22-36. In some examples, the heterologous promoter is a promoter of GPAT5 gene, which is operably linked to the isolated nucleic acid molecule encoding a MYB transcription factor whose amino acid sequence that is selected from the group consisting of SEQ ID NOs: 14 and 22-36. In some examples, the heterologous promoter is a promoter of RALPH gene, which is operably linked to the isolated nucleic acid molecule encoding a MYB transcription factor whose amino acid sequence that is selected from the group consisting of SEQ ID NOs: 14 and 22-36. In some examples, the heterologous promoter is a promoter of MYB84 gene, which is operably linked to the isolated nucleic acid molecule encoding a MYB transcription factor whose amino acid sequence that is selected from the group consisting of SEQ ID NOs: 14 and 22-36.

### IV. MYB41 Orthologs

Myb transcription factors are widespread in animals, plants and fungi. They have been implicated in a wide variety of plant-specific responses, including secondary metabolism, cell shape determination, cell differentiation and stress responses. AtMYB41 from Arabidopsis (Arabidopsis thaliana) has been described as a gene transcriptionally regulated in response to salinity, desiccation, cold and abscisic acid. Lippold et al. (Plant Physiology, April 2009, Plant Physiology, 149:1761-1772) further characterized the gene by subjecting independently AtMYB41-overexpressing lines to detailed transcriptome and metabolome analysis. Their molecular data indicated that the gene is involved in distinct cellular processes, including control of primary metabolism and negative regulation of short-term transcriptional responses to osmotic stress.

Kosma et al. (The Plant Journal, 2014, 80:216-229) showed that overexpression of AtMYB41 can activate the steps necessary for aliphatic suberin synthesis and deposition of cell wall-associated suberin-like lamellae in both epidermal and mesophyll cells of leaves of both Arabidopsis thaliana and Nicotiana benthamiana. While the exact biological function remained unclear to the authors, their evidence suggested that this transcription factor plays a role in augmenting aliphatic suberization under conditions of abiotic stress.

Fatty acyl-CoA reductase (FAR) is known to catalyze the generation of primary fatty alcohols by the reduction of fatty acids in suberin biosynthesis. Wei et al. (Horticulture Research, 2020, 7:86, 10 pages) isolated FAR from kiwifruit (Actinidia chinensis) and transiently overexpressed the isolated AchnFAR in tobacco (Nicotiana benthamiana) leaves. Their studies identified the positive role of transcription factors, including AchnMY41, in the regulation of AchnFAR.

In accordance with the present disclosure, the AtMYB41 gene and its many orthologs when under the control of appropriate promoters may be useful for facilitating the construction of crop plants that have increased or enhanced suberin levels when comparted to appropriate check or control plants. Thus, the heterologous promoter-MYB41 nucleic acid sequences can be used in breeding programs. See, for example, Gentzbittel et al. (1998, Theor. Appl. Genet. 96:519-523). The sequences may also be used to modulate plant development processes, such as metabolic responses to osmotic stress, regulation of FAR, and protecting the plants from other environmental stresses. See, generally, Sambrook et al. (1989) Molecular Cloning: A Laboratory Manual (2nd ed., Cold Spring Harbor Laboratory Press, Plainview, N.Y.). The sequences of the present disclosure can also be used to generate variants (e.g., by ‘domain swapping’) for the generation of new plant types with increased or enhanced suberin levels, particularly in root cells, structures and tissues.

The disclosure encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified polynucleotide or polypeptide is substantially free of other cellular material, or culture medium when produced by recombinant techniques, or substantially free of chemical precursors or other chemicals when chemically synthesized. Suitably, an “isolated” polynucleotide is free of sequences (especially protein encoding sequences) that naturally flank the polynucleotide (i.e., sequences located at the 5′ and 3′ ends of the polynucleotide) in the genomic DNA of the organism from which the polynucleotide was derived. For example, in various examples, the isolated polynucleotide can contain less than about 5 kb, 4 kb, 3 kb, 2 kb, 1 kb, 0.5 kb, or 0.1 kb of nucleotide sequences that naturally flank the polynucleotide in genomic DNA of the cell from which the polynucleotide was derived. A polypeptide that is substantially free of cellular material includes preparations of protein having less than about 30%, 20%, 10%, 5%, (by dry weight) of contaminating protein. When the protein of the disclosure or biologically active portion thereof is recombinantly produced, culture medium suitably represents less than about 30%, 20%, 10%, or 5% (by dry weight) of chemical precursors or non-protein-of-interest chemicals.

The Arabidopsis thaliana MYB41 gene (AtMYB41) is 1,497 nucleic acid base pairs (bp) long, including the 5′ and 3′ UTRs (Sequence Name: AT4G28110.1; Tair Accession: 1009098575; GenBank Accession: NM_118951; arabidopsis.org/servlets/TairObject?type=sequence&id=1002502859; SEQ ID NO: 232).

One exemplary Arabidopsis MYB41 gene used in the examples of the present disclosure is that of SEQ ID NO: 13, which is 1,216 nucleic acid base pairs (bp) long, including 3 exons and 2 introns. The corresponding amino acid sequence is provided in SEQ ID NO: 14. This arabidopsis MYB41 protein sequence is 282 amino acids long (not including stop codon). Also, the complementary DNA (cDNA) of AtMYB41 gene, which codes for AtMYB41 protein, is provided in SEQ ID NO: 15 (AtMYB41 CDS). This AtMYB41 CDS can be used as a replacement of SEQ ID NO: 13 (AtMYB41 gene) for MYB41 expression, after the stop codon sequence (TAA) is removed. Depending on the cloning method used, the stop codon sequence can be included.

A portion of a MYB41 nucleotide sequence that encodes a biologically active portion of a MYB41 polypeptide of the disclosure will encode at least about 5, 6, 7, 8, 9, 10, 11, 12, 13, 14, 15, 16, 17, 18, 19, 20, 21, 22, 23, 24, 25, 30, 40, 50, 60, 70, 80, 90, 100, 120, 150, 300, 400, 500, 600, 700, 800, 900 or 1000 contiguous amino acid residues, or almost up to the total number of amino acids present in a full-length MYB41 polypeptide of the disclosure (for example, 282 amino acid residues for SEQ ID NO: 14). Portions of a MYB41 nucleotide sequence and/or upstream and downstream of the MYB41 gene that are useful as hybridization probes or PCR primers generally need not encode a biologically active portion of a MYB41 polypeptide.

Thus, a portion of a MYB41 nucleotide sequence may encode a biologically active portion of a MYB41 polypeptide, or it may be a fragment that can be used as a hybridization probe or PCR primer using standard methods known in the art. A biologically active portion of a MYB41 polypeptide can be prepared by isolating a portion of one of the MYB41 nucleotide sequences of the disclosure, expressing the encoded portion of the MYB41 polypeptide (e.g., by recombinant expression in vitro), and assessing the activity of the encoded portion of the MYB41 polypeptide.

Nucleic acid molecules that are portions of an MYB41 nucleotide sequence comprise at least about 15, 16, 17, 18, 19, 20, 25, 30, 50, 75, 100, 150, 200, 250, 300, 350, 400, 450, 500, 550, 600, 650, 700, 750, 800, 850, 900, 950, 1000, 1050, 1100, 1150, 1200, 1250, or 1300 nucleotides, or almost up to the number of nucleotides present in a full-length MYB41 nucleotide sequence disclosed herein (for example, about from 150 to 1300 nucleotides for SEQ ID NO: 13).

The disclosure also contemplates using variants of the disclosed nucleotide sequences. Nucleic acid variants can be naturally occurring, such as allelic variants (same locus), homologues (different locus), and orthologues (different organism) or can be non-naturally occurring. Naturally occurring variants such as these can be identified with the use of well-known molecular biology techniques, as, for example, with polymerase chain reaction (PCR) and hybridization techniques as known in the art. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of one of the MYB41 polypeptides of the disclosure. Variant nucleotide sequences also include synthetically derived nucleotide sequences, such as those generated, for example, by using site-directed mutagenesis but which still encode a MYB41 polypeptide of the disclosure. Generally, variants of a particular nucleotide sequence of the disclosure will have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence as determined by sequence alignment programs described elsewhere herein using default parameters.

Variant nucleotide sequences also encompass sequences derived from a mutagenic or recombinant procedures such as ‘DNA shuffling’ which can be used for swapping domains in a polypeptide of interest with domains of other polypeptides. With DNA shuffling, one or more different MYB41 coding sequences can be manipulated to create a new MYB41 sequence possessing desired properties. In this procedure, libraries of recombinant polynucleotides are generated from a population of related polynucleotides comprising sequence regions that have substantial sequence identity and can be homologously recombined in vitro or in vivo. For example, using this approach, sequence motifs encoding a domain of interest may be shuffled between the MYB41 gene of the disclosure and other known MYB41 genes to obtain a new gene coding for a protein with an improved property of interest, such increasing suberin content of plant cells, plant tissues, plant parts and whole plants. Strategies for DNA shuffling are known in the art. See, for example: Stemmer (1994, Proc. Natl. Acad. Sci. USA 91:10747-10751; 1994, Nature 370:389-391); Crameri et al. (1997, Nature Biotech. 15:436-438); Moore et al. (1997, J. Mol. Biol. 272:336-347); Zlang et al. (1997 Proc. Natl. Acad. Sci. USA 94:450-44509); Crameri et al. (1998, Nature 391:288-291); and U.S. Pat. Nos. 5,605,793 and 5,837,458.

The present disclosure provides nucleotide sequences comprising at least a portion of the isolated proteins encoded by nucleotide sequences for MYB41, homologs of MYB41, orthologs of MYB41, paralogs of MYB41, and fragments and variations thereof.

In some examples, the present disclosure provides a nucleotide sequence encoding MYB41, and/or functional fragments and variations thereof comprising a nucleotide sequence that shares at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, about 99.1%, about 99.2%, about 99.3%, about 99.4%, about 99.5%, about 99.6%, about 99.7%, about 99.8%, or about 99.9% sequence identity to any one of SEQ ID NOs: 92-201. In some examples, a nucleotide sequence encoding MYB41 has the nucleic acid sequence of any one of SEQ ID NOs: 92-201.

In some examples, the present disclosure provides nucleotide sequences for MYB41, homologs of MYB41, orthologs of MYB41, paralogs of MYB41, and fragments and variations thereof comprising nucleotide sequences that share at least about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, about 99.1%, about 99.2%, about 99.3%, about 99.4%, about 99.5%, about 99.6%, about 99.7%, about 99.8%, or about 99.9% sequence identity to any one of SEQ ID NOs: 92-201.

In some examples, nucleotide sequences for MYB41, homologs of MYB41, orthologs of MYB41, paralogs of MYB41, and fragments and variations thereof can be used to be expressed in plants. In some examples, said nucleotide sequences can be used to be incorporated into an expression cassette, which is capable of directing expression of a nucleotide sequence for MYB41, homologs of MYB41, orthologs of MYB41, paralogs of MYB41, and fragments and variations thereof in a plant cell, plant tissue, plant part or whole plant. This expression cassette comprises a promoter operably linked to the nucleotide sequence of interest (i.e. MYB41, orthologs of MYB41, and fragments and variations thereof) which is operably linked to termination signals. It also typically comprises sequences required for proper translation of the nucleotide sequence. The coding region usually codes for a protein of interest, (i.e. MYB41). In some examples, the expression cassette comprising the nucleotide sequence for MYB41, homologs of MYB41, orthologs of MYB41, paralogs of MYB41, and fragments and variations thereof is chimeric so that at least one of its components is heterologous with respect to at least one of its other components.

In other examples, the expression cassette is one which is naturally occurring but has been obtained in a recombinant form useful for heterologous expression. The expression of the nucleotide sequence in the expression cassette can be under the control of a constitutive promoter or of an inducible promoter which initiates transcription only when the host cell is exposed to some particular external stimulus. Also, the expression of the nucleotide sequence in the expression cassette can be under the control of a tissue-specific promoter, such as specific root tissues, including, but not limited to, the phellogen, pericycle or procambium. In the case of a multicellular organism, the promoter can also be specific to a particular tissue or organ or stage of development in animal and/or plant.

The present disclosure provides polypeptides and amino acid sequences comprising at least a portion of the proteins encoded by nucleotide sequences for MYB41, homologs of MYB41, orthologs of MYB41, homeologs of MYB41, paralogs of MYB41, and fragments and variations thereof.

The present disclosure provides orthologs of Arabidopsis MYB41 in other plant species. Table 2 presents 55 MYB41 orthologs identified by a conventional ortholog detection method using Orthofinder, which is described in Emms and Kelly (2019), OrthoFinder: phylogenetic orthology inference for comparative genomics, Genome Biology volume 20, Article number: 238. Out of those 55 MYB41 orthologs identified by Orthofinder, genomic synteny-based ortholog detection method identified 11 MYB41 orthologs, as displayed by asterisk (*).

TABLE 2

| Common Name | | SEQ ID NO | SEQ ID NO | SEQ ID NO |
|---|---|---|---|---|
| (Scientific Name) | Gene ID | (Protein) | (CDS**) | (Gene***) |
| Canola | BnaA01T0035200WE | 37 | 92 | 147 |
| (Brassica napus) | BnaA01T0094800WE* | 38* | 93* | 148* |
| | BnaA03T0467400WE | 39 | 94 | 149 |
| | BnaA03T0508300WE* | 40* | 95* | 150* |
| | BnaA08T0104100WE | 41 | 96 | 151 |
| | BnaA09T0219600WE | 42 | 97 | 152 |
| | BnaC01T0113900WE | 43 | 98 | 153 |
| | BnaC01T0168900WE* | 44* | 99* | 154* |
| | BnaC07T0381400WE | 45 | 100 | 155 |
| | BnaC07T0423900WE* | 46* | 101* | 156* |
| | BnaC09T0061000WE | 47 | 102 | 157 |
| | BnaC09T0251300WE | 48 | 103 | 158 |
| Soybean | Glyma.06G299300* | 49* | 104* | 159* |
| (Glycine max) | Glyma.07G189300 | 50 | 105 | 160 |
| | Glyma.08G059900 | 51 | 106 | 161 |
| | Glyma.12G106400* | 52* | 107* | 162* |
| | Glyma.12G199600 | 53 | 108 | 163 |
| | Glyma.13G247200 | 54 | 109 | 164 |
| | Glyma.13G302400 | 55 | 110 | 165 |
| | Glyma.15G066800 | 56 | 111 | 166 |
| Cotton | Gohir.A01G069600.1.v2.1 | 57 | 112 | 167 |
| (Gossypium hirsutum) | Gohir.A04G106300.1.v2.1 | 58 | 113 | 168 |
| | Gohir.A05G166300.1.v2.1 | 59 | 114 | 169 |
| | Gohir.A06G122800.1.v2.1 | 60 | 115 | 170 |
| | Gohir.A10G021900.1.v2.1 | 61 | 116 | 171 |
| | Gohir.A12G136100.1.v2.1 | 62 | 117 | 172 |
| | Gohir.D01G056875.1.v2.1 | 63 | 118 | 173 |
| | Gohir.D04G146300.1.v2.1 | 64 | 119 | 174 |
| | Gohir.D05G169300.1.v2.1 | 65 | 120 | 175 |
| | Gohir.D06G127600.1.v2.1 | 66 | 121 | 176 |
| | Gohir.D10G022700.1.v2.1 | 67 | 122 | 177 |
| | Gohir.D12G140100.1.v2.1 | 68 | 123 | 178 |
| Rice | LOC_Os07g37210 | 69 | 124 | 179 |
| (Oryza sativa) | | | | |
| Lotus | LotjaGi3g1v0439100* | 70* | 125* | 180* |
| (Lotus japonicus) | LotjaGi4g1v0048000 | 71 | 126 | 181 |
| | LotjaGi6g1v0339900 | 72 | 127 | 182 |
| Radish | RSAskr1.0R0g16937 | 73 | 128 | 183 |
| (Raphanus | RSAskr1.0R1g26405 | 74 | 129 | 184 |
| sativus) | RSAskr1.0R2g34195 | 75 | 130 | 185 |
| | RSAskr1.0R2g34898* | 76* | 131* | 186* |
| | RSAskr1.0R2g34992* | 77* | 132* | 187* |
| | RSAskr1.0R4g43022* | 78* | 133* | 188* |
| | RSAskr1.0R4g43654 | 79 | 134 | 189 |
| | RSAskr1.0R8g79941 | 80 | 135 | 190 |
| | RSAskr1.0R9g86808 | 81 | 136 | 191 |
| Setaria | Seita.2G351400.1.v2.2 | 82 | 137 | 192 |
| (Setaria italica) | | | | |
| Sorghum | Sobic.002G337800 | 83 | 138 | 193 |
| (Sorghum bicolor) | | | | |
| Pennycress | Ta1014.a04.1.g37760 | 84 | 139 | 194 |
| (Thlaspi arvense) | Ta1014.a04.4.g35360 | 85 | 140 | 195 |
| | Ta1014.a04.4.g43820* | 86* | 141* | 196* |
| Southern cattail | TD08a01.chr_3.g00580 | 87 | 142 | 197 |
| (Typha domingensis) | | | | |
| Wheat | TraesCS2A03G0434000.1 | 88 | 143 | 198 |
| (Triticum aestivum) | | | | |
| | TraesCS2D03G0437900.1 | 89 | 144 | 199 |
| Maize | Zm00001d006585_T001.RefGen_V4 | 90 | 145 | 200 |
| (Zea mays) | Zm00001d021761_T001.RefGen_V4 | 91 | 146 | 201 |

*MYB41 ortholog also identified by synteny-based analysis
**CDS: Coding Sequence, which is a region of DNA whose sequence determines sequence of amino acids in a protein
***Gene sequence includes 5′ and 3′ UTRs

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence set forth in any one of SEQ ID NOs: 14 and 22-36.

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence set forth in any one of SEQ ID NOs: 37-91.

Table 3 shows the amino acid sequence identity between AtMYB41 (SEQ ID NO: 14) and the 11 MYB41 orthologs identified using both Orthofinder and synteny-based analysis.

TABLE 3

| | | | Amino Acid Sequence |
|---|---|---|---|
| Common Name | | Protein Sequence | Identity to SEQ ID NO: 14 |
| (Scientific Name) | Gene ID | (SEQ ID NO) | (AtMYB41) |
| Canola | BnaA01T0094800WE* | SEQ ID NO: 38* | 83.27% |
| (Brassica napus) | BnaA03T0508300WE* | SEQ ID NO: 40* | 82.37% |
| | BnaC01T0168900WE* | SEQ ID NO: 44* | 82.14% |
| | BnaC07T0423900WE* | SEQ ID NO: 46* | 82.37% |
| Soybean | Glyma.06G299300* | SEQ ID NO: 61* | 56.00% |
| (Glycine max) | Glyma.12G106400* | SEQ ID NO: 64* | 58.36% |
| Lotus | LotjaGi3g1v0439100* | SEQ ID NO: 69* | 56.83% |
| (Lotus japonicus) | | | |
| Radish | RSAskr1.0R2g34898* | SEQ ID NO: 76* | 82.86% |
| (Raphanus sativus) | RSAskr1.0R2g34992* | SEQ ID NO: 77* | 82.14% |
| | RSAskr1.0R4g43022* | SEQ ID NO: 78* | 82.55% |
| Pennycress | Ta1014.a04.4.g43820* | SEQ ID NO: 88* | 87.36% |
| (Thlaspi arvense) | | | |

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence set forth in any one of SEQ ID NOs: 38, 40, 44, 46, 49, 52, 70, 76, 77, 78, and 86.

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence encoded by nucleic acid sequences of MYB41 gene orthologs set forth in any one of SEQ ID NOs: 92-146.

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence encoded by nucleic acid sequences of MYB41 gene orthologs set forth in any one of SEQ ID NOS: 93, 95, 99, 101, 104, 107, 125, 131, 132, 133, and 141.

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence encoded by nucleic acid sequences of MYB41 gene orthologs set forth in any one of SEQ ID NOs: 147-201.

In some examples, the present disclosure provides an isolated polypeptide comprising an amino acid sequence that shares at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or 100% identity to an amino acid sequence encoded by nucleic acid sequences of MYB41 gene orthologs set forth in any one of SEQ ID NOs: 148, 150, 154, 156, 159, 162, 180, 186, 187, 188, and 196.

The disclosure also provides variants and fragments of MYB41 protein orthologs set forth in SEQ ID NOs: 37-91. The disclosure also provides variants and fragments of MYB41 protein orthologs set forth in SEQ ID NOs: 38, 40, 44, 46, 61, 64, 69, 76, 77, 78, and 88.

The disclosure also provides variants and fragments of MYB41 protein orthologs encoded by CDS sequences set forth in SEQ ID NOs: 92-146. The disclosure also provides variants and fragments of MYB41 protein orthologs encoded by CDS sequences set forth in SEQ ID NOs: 93, 95, 99, 101, 104, 107, 125, 131, 132, 133, and 141.

The disclosure also provides variants and fragments of MYB41 protein orthologs encoded by genomic sequences set forth in SEQ ID NOs: 147-201. The disclosure also provides variants and fragments of MYB41 protein orthologs encoded by gene sequences set forth in SEQ ID NOs: 148, 150, 154, 156, 159, 162, 180, 186, 187, 188, and 196.

The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both.

The present disclosure also provides an amino acid sequence encoded by the nucleic acid sequences of MYB 41 orthologs (including SEQ ID NOs: 92-146 and 147-201), and/or fragments and variations thereof. In some examples, the present disclosure provides an isolated polypeptide comprising an amino acid sequence that shares at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence encoded by the nucleic acid sequences of SEQ ID NOs: 92-146 (CDS of crop MYB41 orthologs) and 147-201 (genomic sequence of crop MYB41 orthologs).

In one example, the present disclosure provides an isolated polypeptide comprising an amino acid sequence that shares at least about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to an amino acid sequence encoded by the nucleic acid sequences of MYB41, homologs of MYB41, orthologs of MYB41, paralogs of MYB41, and/or fragments and variations thereof.

The disclosure also encompasses variants and fragments of proteins of an amino acid sequence encoded by the nucleic acid sequences of MYB41, homologs of MYB41, orthologs of MYB41 and/or paralogs of MYB41. The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both.

Functional fragments and variants of a polypeptide include those fragments and variants that maintain one or more functions of the parent polypeptide. It is recognized that the gene or cDNA encoding a polypeptide can be considerably mutated without materially altering one or more of the polypeptide's functions. First, the genetic code is well-known to be degenerate, and thus different codons encode the same amino acids. Second, even where an amino acid substitution is introduced, the mutation can be conservative and have no material impact on the essential function(s) of a protein. See, e.g., Stryer Biochemistry 3rd Ed., 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al. J. Immunol. 159 (5): 2502-12, 1997). Other modifications that can be made without materially impairing one or more functions of a polypeptide can include, for example, in vivo or in vitro chemical and biochemical modifications or the incorporation of unusual amino acids. Such modifications include, but are not limited to, for example, acetylation, carboxylation, phosphorylation, glycosylation, ubiquination, labelling, e.g., with radionucleotides, and various enzymatic modifications. A variety of methods for labelling polypeptides, and labels useful for such purposes, are well known in the art, and include radioactive isotopes such as 32P, ligands which bind to or are bound by labelled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues. These mutations can be natural or purposely changed. In some examples, mutations containing alterations that produce silent substitutions, additions, or deletions, but do not alter the properties or activities of the proteins or how the proteins are made are an example of the disclosure.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751 757, 1987), O'Regan et al. (Gene, 77:237 251, 1989), Sahin Toth et al. (Protein Sci., 3:240 247, 1994), Hochuli et al. (Bio/Technology, 6:1321 1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions.

TABLE 4
Exemplary conservative amino acid substitutions

| | | Highly Conserved | Conserved |
|---|---|---|---|
| | Very Highly - | Substitutions | Substitutions |
| Original | Conserved | (from the Blosum90 | (from the Blosum65 |
| Residue | Substitutions | Matrix) | Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, | Arg, Asp, Gln, Glu, His, |
| | | Ser, Thr | Lys, Ser, Thr |
| Asp | Glu | Asn, Glu | Asn, Gln, Glu, Ser |
| Cys | Ser | None | Ala |
| Gln | Asn | Arg, Asn, Glu, His, | Arg, Asn, Asp, Glu, His, |
| | | Lys, Met | Lys, Met, Ser |
| Glu | Asp | Asp, Gln, Lys | Arg, Asn, Asp, Gln, His, |
| | | | Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, |
| | | | Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some examples, variants can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a variant functionally similar to the disclosed an amino acid sequences encoded by the nucleic acid sequences of MYB41, homologs of MYB41, orthologs of MYB41 and/or paralogs of MYB41, and/or fragments and variations thereof.

In some examples, variants may differ from the disclosed sequences by alteration of the coding region to fit the codon usage bias of the particular organism into which the molecule is to be introduced. In other examples, the coding region may be altered by taking advantage of the degeneracy of the genetic code to alter the coding sequence such that, while the nucleotide sequence is substantially altered, it nevertheless encodes a protein having an amino acid sequence substantially similar to the disclosed an amino acid sequences encoded by the nucleic acid sequences of MYB41, homologs of MYB41, orthologs of MYB41 and/or paralogs of MYB41, and/or fragments and variations thereof.

In some examples, functional fragments derived from the MYB41 orthologs of the present disclosure are provided. The functional fragments can still confer the ability to increase suberin content in plant cells, plant tissues, plant parts and whole plants when expressed in a plant. In some examples, the functional fragments contain at least the conserved region or Bowman-Birk inhibitor domain of a wild type MYB41 orthologs, or functional variants thereof. In some examples, the functional fragments contain one or more conserved region shared by two or more MYB41 orthologs, shared by two or more MYB41 orthologs in the same plant genus, shared by two or more dicot MYB41 orthologs, and/or shared by two or more monocot MYB41 orthologs. The conserved regions or Bowman-Birk inhibitor domains can be determined by any suitable computer program, such as NCBI protein BLAST program and NCBI Alignment program, or equivalent programs. In some examples, the functional fragments are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids shorter compared to the MYB41 orthologs of the present disclosure. In some examples, the functional fragments are made by deleting one or more amino acid of the MYB41 orthologs of the present disclosure. In some examples, the functional fragments share at least 80%, 85%, 90%, 95%, 96%, 97%, 98%, 99%, or more identity to the MYB41 orthologs of the present disclosure.

In some examples, functional chimeric or synthetic polypeptides derived from the MYB41 orthologs of the present disclosure are provided. The functional chimeric or synthetic polypeptides can still confer the ability to increase suberin content when expressed in a plant. In some examples, the functional chimeric or synthetic polypeptides contain at least the conserved region or Bowman-Birk inhibitor domain of a wild type MYB41 orthologs, or functional variants thereof. In some examples, the functional chimeric or synthetic polypeptides contain one or more conserved region shared by two or more MYB41 orthologs, shared by two or more MYB41 orthologs in the same plant genus, shared by two or more monocot MYB41 orthologs, and/or shared by two or more dicot MYB41 orthologs. The conserved regions or Bowman-Birk inhibitor domains can be determined by any suitable computer program, such as NCBI protein BLAST program and NCBI Alignment program, or equivalent programs. In some examples, the functional chimeric or synthetic polypeptides share at least 80%, 85%, 90%, 95%, 96%, 97%, 98%, 99%, or more identity to the MYB41 orthologs of the present disclosure.

Sequences of conserved regions unique to FW-sensitive alleles can also be used to knock-down the level of one or more MYB41 orthologs. In some examples, sequences of conserved regions can be used to make gene silencing molecules to target one or more MYB41 orthologs. In some examples, the gene silencing molecules are selected from the group consisting of double-stranded polynucleotides, single-stranded polynucleotides or Mixed Duplex Oligonucleotides. In some examples, the gene silencing molecules comprises a DNA/RNA fragment of about 10 bp, 15 bp, 19 bp, 20 bp, 21 bp, 25 bp, 30 bp, 40 bp, 50 bp, 60 bp, 70 bp, 80 bp, 90 bp, 100 bp, 150 bp, 200 pb, 250 bp, 300 bp, 350 bp, 400 bp, 500 bp, 600 bp, 700 bp, 800 bp, 900 bp, 1000 bp, or more polynucleotides, wherein the DNA/RNA fragment share at least 90%, 95%, 99%, or more identity to a conserved region of the MYB41 orthologs sequences of the present disclosure, or complementary sequences thereof.

The present disclosure provides an isolated nucleic acid sequence comprising a sequence selected from the group consisting of MYB41, homologs of MYB41, orthologs of MYB41, paralogs of MYB41, and fragments and variations thereof.

In some examples, the present disclosure provides an isolated polynucleotide comprising a nucleic acid sequence that shares at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to a nucleic acid of MYB41 orthologs, set forth in SEQ ID NOs: 92-201.

In some examples, the present disclosure provides an isolated polynucleotide comprising a nucleic acid sequence that shares at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to a nucleic acid of MYB41 orthologs, set forth in SEQ ID NOs: 93, 95, 99, 101, 104, 107, 125, 131, 132, 133, 141, 148, 150, 154, 156, 159, 162, 180, 186, 187, 188, and 196.

In some examples, the present disclosure provides an isolated polypeptide comprising an amino acid sequence that shares at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to an amino acid of MYB41 orthologs, set forth in SEQ ID NOs: 37-91.

In some examples, the present disclosure provides an isolated polypeptide comprising an amino acid sequence that shares at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% or 100% identity to an amino acid of MYB41 orthologs, set forth in SEQ ID NOs: 38, 40, 44, 46, 49, 52, 70, 76, 77, 78, and 86.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The present disclosure also provides a chimeric gene including the isolated nucleic acid sequence of any one of the polynucleotides described above operably linked to suitable regulatory sequences. In some examples, a chimeric gene comprises the isolated nucleic acid sequence comprising a sequence selected from the group consisting of MYB41, homologs of MYB41, orthologs of MYB41, paralogs of MYB41, and fragments and variations thereof.

In some examples, a chimeric gene includes a nucleic acid sequence set forth in SEQ ID NOs: 92-146, which are CDS sequences of MYB41 orthologs in Canola (Brassica napus), Soybean (Glycine max), Cotton (Gossypium hirsutum), Rice (Oryza sativa), Lotus (Lotus japonicus), Radish (Raphanus sativus), Setaria (Setaria italica), Sorghum (Sorghum bicolor), Pennycress (Thlaspi arvense), Southern cattail (Typha domingensis), Wheat (Triticum aestivum), and Maize (Zea mays).

In some examples, a chimeric gene includes a nucleic acid sequence set forth in SEQ ID NOs: 93, 95, 99, 101, 104, 107, 125, 131, 132, 133, and 141, which are CDS sequences of MYB41 orthologs in canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

In some examples, a chimeric gene comprises a nucleic acid sequence set forth in SEQ ID NOs: 147-201, which are genomic sequences of MYB41 orthologs in canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

In some examples, a chimeric gene comprises a nucleic acid sequence set forth in SEQ ID NOs: 148, 150, 154, 156, 159, 162, 180, 186, 187, 188, and 196, which are genomic sequences of MYB41 orthologs in canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

In some examples, a chimeric gene comprises a nucleic acid sequence encoding an amino acid sequence set forth in SEQ ID NOs: 37-91, which are protein sequence sequences of MYB41 orthologs in canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

In some examples, a chimeric gene comprises a nucleic acid sequence encoding an amino acid sequence set forth in SEQ ID NOs: 38, 40, 44, 46, 49, 52, 70, 76, 77, 78, and 86., which are protein sequence sequences of MYB41 orthologs in canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

In some examples, the MYB41 amino acid sequence is present in Canola (Brassica napus) and comprises any one of SEQ ID NOs: 37-48 or a sequence at least 80% identical thereto. In some examples, the MYB41 amino acid sequence is present in soybean (Glycine max) and comprises any one of SEQ ID NOs: 49-56 or a sequence at least 80% identical thereto. In some examples, the MYB41 amino acid sequence is present in cotton (Gossypium hirsutum) and comprises any one of SEQ ID NOs: 57-68 or a sequence at least 80% identical thereto. In some examples, the MYB41 amino acid sequence is present in rice (Oryza sativa) and comprises any one of SEQ ID NO: 69 or a sequence at least 80% identical thereto. In some examples, the MYB41 amino acid sequence is present in lotus (Lotus japonicus) and comprises any one of SEQ ID NOs: 70-72 or a sequence at least 80% identical thereto. In some examples, the MYB41 amino acid sequence is present in radish (Raphanus sativus) and comprises any one of SEQ ID NOs: 73-81 or a sequence at least 80% identical thereto. In some examples, the MYB41 amino acid sequence is present in setaria (Setaria italica) and comprises any one of SEQ ID NO: 82 or a sequence at least 80% identical thereto. In some examples, the MYB41 amino acid sequence is present in sorghum (Sorghum bicolor) and comprises any one of SEQ ID NO: 83 or a sequence at least 80% identical thereto. In some examples, the MYB41 amino acid sequence is present in pennycress (Thlaspi arvense) and comprises any one of SEQ ID NOs: 84-86 or a sequence at least 80% identical thereto. In some examples, the MYB41 amino acid sequence is present in Southern cattail (Typha domingensis) and comprises any one of SEQ ID NO: 87 or a sequence at least 80% identical thereto. In some examples, the MYB41 amino acid sequence is present in wheat (Triticum aestivum) and comprises any one of SEQ ID NOs: 88-89 or a sequence at least 80% identical thereto. In some examples, the MYB41 amino acid sequence is present in maize (Zea mays) and comprises any one of SEQ ID NOs: 90-91 or oa sequence at least 80% identical thereto.

In some examples, the MYB41 amino acid sequence comprises at least 80% identical to any one of SEQ ID NOs: 38, 40, 44, 46, 49, 52, 70, 76, 77, 78, and 86. In some examples, the nucleic acid sequence comprises at least 80% identical to any one of SEQ ID NOs: 93, 95, 99, 101, 104, 107, 125, 131, 132, 133, and 141. In some examples, the nucleic acid sequence comprises at least 80% identical to any one of SEQ ID NOs: 148, 150, 154, 156, 159, 162, 180, 186, 187, 188, and 196.

In some examples, a plant, a part thereof, or a cell thereof, comprising the isolated nucleic acid molecule taught herein. In some examples, a plant, a part thereof, or a cell thereof, comprising the transformation vector taught herein.

The present disclosure provides a genetically engineered plant, comprising an isolated nucleic acid molecule encoding a MYB41 amino acid sequence that is selected from the group consisting of SEQ ID NOs: 38, 40, 44, 46, 49, 52, 70, 76, 77, 78, and 86, operably linked to a nucleic acid sequence encoding a heterologous promoter, wherein expression of the isolated nucleic acid molecule in a plant results in increased levels of suberin as compared to wild-type check plants lacking the isolated nucleic acid molecule. The present disclosure also provides a genetically engineered plant, comprising an isolated nucleic acid molecule that is selected from the group consisting of SEQ ID NOs: 93, 95, 99, 101, 104, 107, 125, 131, 132, 133, 141 148, 150, 154, 156, 159, 162, 180, 186, 187, 188, and 196, operably linked to a nucleic acid sequence encoding a heterologous promoter, wherein expression of the isolated nucleic acid molecule in a plant results in increased levels of suberin as compared to wild-type check plants lacking the isolated nucleic acid molecule.

In some examples, the genetically engineered plant is canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

In some examples, the heterologous promoter is a promoter of FACT gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86. In some examples, the heterologous promoter is a promoter of HORST gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86. In some examples, the heterologous promoter is a promoter of ASFT gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86. In some examples, the heterologous promoter is a promoter of GPAT5 gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86. In some examples, the heterologous promoter is a promoter of RALPH gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86. In some examples, the heterologous promoter is a promoter of MYB84 gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.

### V. Promoter Sequences

As set forth herein, the inventors discovered that certain promoters operably-linked to a MYB41 gene and transformed into plant enable those transgenic plants to develop additional periderm layers at an earlier stage of root development and/or deposit more suberin in periderm cells without negatively impacting plant health.

Bevan and Walsh (The Arabidopsis genome: A foundation for plant research, 2005, Genome Research, 15:1632-1642) concluded that the sequencing of the Arabidopsis and rice genomes provide a strong platform for supporting integrative plant science across model and crop species (page 1639, second column, Perspectives). Promoters isolated from Arabidopsis can drive gene expression in important crops species. For example, Jiang et al. (Characterization of a strong and constitutive promoter from the Arabidopsis serine carboxypeptidase-like gene AtSCPL30 as a potential tool for crop transgenic breeding, 2018, BMC Biotechnology, 18:59, 13 pages) isolated a full-length promoter (PD1) from Arabidopsis and demonstrated it conferred strong and constitutive expression of transgenes in almost all tissues and development stages of Nicotiana benthamiana transgenic plants. Drought responsive promoters HVA22E and PLDdelta identified and isolated from Arabidopsis thaliana drove transgenic gene expression when used to transform corn and soybeans (U.S. Pat. Nos. 7,632,982 and 8,692,071). Two seed-specific promoters isolated from Arabidopsis (i.e., AtS1 and AtS3) conferred seed-specific accumulation of GUS activity in both transgenic Arabidopsis and transgenic tobacco (U.S. Pat. No. 6,100,450).

Exemplary promoters are provided in the following table. For the promoters presented in Table 5, GUS expression was observed in the periderm and endodermis. Weak GUS expression was also observed in some cortex and in some epidermis cells. Except for proMYB84, no GUS expression was detected in the rosette leaves of the promoters. These results demonstrate that these promoters are specifically root-expressed with no detectable expression in rosette levels at day 14.

TABLE 5
Exemplary Promoters Sequences Driving MYB41 Expression

| | Nucleic Acid | |
|---|---|---|
| Promoter | Sequence | Origin |
| proFACT (aka pFACT) | SEQ ID NO: 1 | Arabidopsis thaliana |
| proHORST (aka pHORST) | SEQ ID NO: 2 | Arabidopsis thaliana |
| proASFT (aka pASFT) | SEQ ID NO: 3 | Arabidopsis thaliana |
| proGPAT5 (aka pGPAT5) | SEQ ID NO: 4 | Arabidopsis thaliana |
| proRALPH (aka pRALPH) | SEQ ID NO: 5 | Arabidopsis thaliana |
| proMYB84 (aka pMYB84) | SEQ ID NO: 6 | Arabidopsis thaliana |

Further exemplary promoters which drive MYB41 expression are provided. In one example, the promoter is a promoter of a FACT ortholog, a HORST ortholog, an ASFT ortholog, a GPAT5 ortholog, a RALPH ortholog and/or a MYB84 ortholog. Promoters can be derived from canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays). The promoter can be a native promoter. The promoter can be a heterologous promoter.

Exemplary promoters of FACT orthologs include: SEQ ID NOs: 234, 239-246, 280-285, 314-316, 340-349, 379, 380, and/or 389-391. The present disclosure provides an isolated nucleic acid molecule comprising an isolated nucleic acid sequence encoding a protein of interest (such as MYB41), which is operably linked to a nucleic acid sequence encoding a heterologous promoter selected from the group comprising or consisting of SEQ ID NOs: 234, 239-246, 280-285, 314-316, 340-349, 379, 380, and 389-391.

Exemplary promoters of HORST orthologs include: SEQ ID NOs: 236, 254-258, 288, 289, 319-321, 352-354, 383, 394, and/or 395. The present disclosure provides an isolated nucleic acid molecule comprising an isolated nucleic acid sequence encoding a protein of interest (such as MYB41), which is operably linked to a nucleic acid sequence encoding a heterologous promoter selected from the group comprising or consisting of SEQ ID NOs: 236, 254-258, 288, 289, 319-321, 352-354, 383, 394, and 395.

Exemplary promoters of ASFT orthologs include: SEQ ID NOs: 233, 239-244, 280-285, 314-316, 340-349, 378, 379, and/or 389-391. The present disclosure provides an isolated nucleic acid molecule comprising an isolated nucleic acid sequence encoding a protein of interest (such as MYB41), which is operably linked to a nucleic acid sequence encoding a heterologous promoter selected from the group comprising or consisting of SEQ ID NOs: 233, 239-244, 280-285, 314-316, 340-349, 378, 379, and 389-391.

Exemplary promoters of GPAT5 orthologs include: SEQ ID NOs: 235, 247-253, 286, 287, 317, 318, 350, 351, 381, 382, 392, and/or 393. The present disclosure provides an isolated nucleic acid molecule comprising an isolated nucleic acid sequence encoding a protein of interest (such as MYB41), which is operably linked to a nucleic acid sequence encoding a heterologous promoter selected from the group comprising or consisting of SEQ ID NOs: 235, 247-253, 286, 287, 317, 318, 350, 351, 381, 382, 392, and 393 . . .

Exemplary promoters of RALPH orthologs include: SEQ ID NOs: 238, 387, and/or 388. The present disclosure provides an isolated nucleic acid molecule comprising an isolated nucleic acid sequence encoding a protein of interest (such as MYB41), which is operably linked to a nucleic acid sequence encoding a heterologous promoter selected from the group comprising or consisting of SEQ ID NOs: 238, 387, and 388.

Exemplary promoters of MYB84 orthologs include: SEQ ID NOs: 237, 259-279, 290-313, 322-339, 355-377, 384-386, and/or 396-411. The present disclosure provides an isolated nucleic acid molecule comprising an isolated nucleic acid sequence encoding a protein of interest (such as MYB41), which is operably linked to a nucleic acid sequence encoding a heterologous promoter selected from the group comprising or consisting of SEQ ID NOs: 237, 259-279, 290-313, 322-339, 355-377, 384-386, and 396-411.

TABLE 7
Exemplary Promoters Sequences Driving MYB41 Expression

| Scientific | | Ortholog or | SEQ |
|---|---|---|---|
| Name: | Gene ID: | Homolog of: | ID NO: |
| Arabidopsis | AT5G41040.Araport11.447 | ASFT | 233 |
| thaliana | AT5G63560.Araport11.447 | FACT | 234 |
| | AT3G11430.Araport11.447 | GPAT5 | 235 |
| | AT5G58860.Araport11.447 | HORST | 236 |
| | AT3G49690.Araport11.447 | MYB84 | 237 |
| | AT5G23190.Araport11.447 | RALPH | 238 |
| Brassica | BnaA04G0115400WE | ASFT, FACT | 239 |
| napus | BnaA06G0281000WE | ASFT, FACT | 240 |
| | BnaA07G0168700WE | ASFT, FACT | 241 |
| | BnaC03G0467600WE | ASFT, FACT | 242 |
| | BnaC04G0400000WE | ASFT, FACT | 243 |
| | BnaC06G0157800WE | ASFT, FACT | 244 |
| | BnaA06G0160600WE | FACT | 245 |
| | BnaC08G0244100WE | FACT | 246 |
| | BnaA01G0287400WE | GPAT5 | 247 |
| | BnaA05G0382400WE | GPAT5 | 248 |
| | BnaA10G0265500WE | GPAT5 | 249 |
| | BnaC01G0395100WE | GPAT5 | 250 |
| | BnaC05G0458200WE | GPAT5 | 251 |
| | BnaC05G0473800WE | GPAT5 | 252 |
| | BnaC09G0558900WE | GPAT5 | 253 |
| | BnaA02G0109300WE | HORST | 254 |
| | BnaA03G0120100WE | HORST | 255 |
| | BnaC02G0124600WE | HORST | 256 |
| | BnaC02G0124700WE | HORST | 257 |
| | BnaC03G0062400WE | HORST | 258 |
| | BnaA02G0117400WE | MYB84 | 259 |
| | BnaA02G0405900WE | MYB84 | 260 |
| | BnaA03G0126300WE | MYB84 | 261 |
| | BnaA03G0190500WE | MYB84 | 262 |
| | BnaA04G0225000WE | MYB84 | 263 |
| | BnaA05G0072000WE | MYB84 | 264 |
| | BnaA06G0152800WE | MYB84 | 265 |
| | BnaA06G0339900WE | MYB84 | 266 |
| | BnaA09G0083600WE | MYB84 | 267 |
| | BnaC02G0134600WE | MYB84 | 268 |
| | BnaC02G0532700WE | MYB84 | 269 |
| | BnaC03G0069300WE | MYB84 | 270 |
| | BnaC03G0144000WE | MYB84 | 271 |
| | BnaC03G0444200WE | MYB84 | 272 |
| | BnaC04G0097800WE | MYB84 | 273 |
| | BnaC04G0537300WE | MYB84 | 274 |
| | BnaC08G0251300WE | MYB84 | 275 |
| | BnaC09G0097800WE | MYB84 | 276 |
| | BnaC09G0384000WE | MYB84 | 277 |
| | Bnascaffold1716G0006000WE | MYB84 | 278 |
| | Bnascaffold286G0011300WE | MYB84 | 279 |
| Glycine max | Glyma.04G198000.Wm82.a4.v1 | ASFT, FACT | 280 |
| | Glyma.05G203000.Wm82.a4.v1 | ASFT, FACT | 281 |
| | Glyma.06G167600.Wm82.a4.v1 | ASFT, FACT | 282 |
| | Glyma.08G010500.Wm82.a4.v1 | ASFT, FACT | 283 |
| | Glyma.16G053300.Wm82.a4.v1 | ASFT, FACT | 284 |
| | Glyma.19G096100.Wm82.a4.v1 | ASFT, FACT | 285 |
| | Glyma.02G249300.Wm82.a4.v1 | GPAT5 | 286 |
| | Glyma.14G067200.Wm82.a4.v1 | GPAT5 | 287 |
| | Glyma.11G175900.Wm82.a4.v1 | HORST | 288 |
| | Glyma.14G192500.Wm82.a4.v1 | HORST | 289 |
| | Glyma.01G004900.Wm82.a4.v1 | MYB84 | 290 |
| | Glyma.01G196200.Wm82.a4.v1 | MYB84 | 291 |
| | Glyma.03G183900.Wm82.a4.v1 | MYB84 | 292 |
| | Glyma.04G048900.Wm82.a4.v1 | MYB84 | 293 |
| | Glyma.05G109200.Wm82.a4.v1 | MYB84 | 294 |
| | Glyma.05G224300.Wm82.a4.v1 | MYB84 | 295 |
| | Glyma.06G050000.Wm82.a4.v1 | MYB84 | 296 |
| | Glyma.07G126900.Wm82.a4.v1 | MYB84 | 297 |
| | Glyma.08G031300.Wm82.a4.v1 | MYB84 | 298 |
| | Glyma.10G054700.Wm82.a4.v1 | MYB84 | 299 |
| | Glyma.11G045400.Wm82.a4.v1 | MYB84 | 300 |
| | Glyma.11G194100.Wm82.a4.v1 | MYB84 | 301 |
| | Glyma.12G079800.Wm82.a4.v1 | MYB84 | 302 |
| | Glyma.12G177700.Wm82.a4.v1 | MYB84 | 303 |
| | Glyma.13G094400.Wm82.a4.v1 | MYB84 | 304 |
| | Glyma.13G141900.Wm82.a4.v1 | MYB84 | 305 |
| | Glyma.13G322900.Wm82.a4.v1 | MYB84 | 306 |
| | Glyma.14G090900.Wm82.a4.v1 | MYB84 | 307 |
| | Glyma.14G091500.Wm82.a4.v1 | MYB84 | 308 |
| | Glyma.17G065800.Wm82.a4.v1 | MYB84 | 309 |
| | Glyma.17G158000.Wm82.a4.v1 | MYB84 | 310 |
| | Glyma.17G231900.Wm82.a4.v1 | MYB84 | 311 |
| | Glyma.17G232220.Wm82.a4.v1 | MYB84 | 312 |
| | Glyma.19G184500.Wm82.a4.v1 | MYB84 | 313 |
| Oryza sativa | LOC_Os11g31090.MSUv7.0 | ASFT, FACT | 314 |
| | OsKitaake01g453900.v3.1 | ASFT, FACT | 315 |
| | OsKitaake11g142400.v3.1 | ASFT, FACT | 316 |
| | LOC_Os05g38350.MSUv7.0 | GPAT5 | 317 |
| | OsKitaake05g185700.v3.1 | GPAT5 | 318 |
| | LOC_Os01g63540.MSUv7.0 | HORST | 319 |
| | OsKitaake01g403900.v3.1 | HORST | 320 |
| | OsKitaake05g178800.v3.1 | HORST | 321 |
| | LOC_Os01g09590.MSUv7.0 | MYB84 | 322 |
| | LOC_Os01g49160.MSUv7.0 | MYB84 | 323 |
| | LOC_Os01g52410.MSUv7.0 | MYB84 | 324 |
| | LOC_Os02g54520.MSUv7.0 | MYB84 | 325 |
| | LOC_Os03g55300.MSUv7.0 | MYB84 | 326 |
| | LOC_Os03g56090.MSUv7.0 | MYB84 | 327 |
| | LOC_Os05g48010.MSUv7.0 | MYB84 | 328 |
| | LOC_Os08g15020.MSUv7.0 | MYB84 | 329 |
| | LOC_Os09g26170.MSUv7.0 | MYB84 | 330 |
| | OsKitaake01g066116.v3.1 | MYB84 | 331 |
| | OsKitaake01g282200.v3.1 | MYB84 | 332 |
| | OsKitaake01g307500.v3.1 | MYB84 | 333 |
| | OsKitaake02g360500.v3.1 | MYB84 | 334 |
| | OsKitaake03g355000.v3.1 | MYB84 | 335 |
| | OsKitaake04g047100.v3.1 | MYB84 | 336 |
| | OsKitaake05g254900.v3.1 | MYB84 | 337 |
| | OsKitaake08g084800.v3.1 | MYB84 | 338 |
| | OsKitaake09g108700.v3.1 | MYB84 | 339 |
| Sorghum | SbiRTX430.01G109800.v2.1 | ASFT, FACT | 340 |
| bicolor | SbiRTX430.03G397000.v2.1 | ASFT, FACT | 341 |
| | SbiRTX430.03G441300.v2.1 | ASFT, FACT | 342 |
| | SbiRTX430.05G136000.v2.1 | ASFT, FACT | 343 |
| | SbiRTX430.07G036700.v2.1 | ASFT, FACT | 344 |
| | SbiRTX430.07G156600.v2.1 | ASFT, FACT | 345 |
| | Sobic.003G368100.v3.2 | ASFT, FACT | 346 |
| | Sobic.003G408900.v3.2 | ASFT, FACT | 347 |
| | Sobic.005G122800.v3.2 | ASFT, FACT | 348 |
| | Sobic.007G144400.v3.2 | ASFT, FACT | 349 |
| | SbiRTX430.09G169900.v2.1 | GPAT5 | 350 |
| | Sobic.009G162000.v3.2 | GPAT5 | 351 |
| | SbiRTX430.03G388900.v2.1 | HORST | 352 |
| | SbiRTX430.09G163400.v2.1 | HORST | 353 |
| | Sobic.003G360600.v3.2 | HORST | 354 |
| | SbiRTX430.01G077000.v2.1 | MYB84 | 355 |
| | SbiRTX430.02G142400.v2.1 | MYB84 | 356 |
| | SbiRTX430.02G214800.v2.1 | MYB84 | 357 |
| | SbiRTX430.03G037400.v2.1 | MYB84 | 358 |
| | SbiRTX430.03G280900.v2.1 | MYB84 | 359 |
| | SbiRTX430.03G302200.v2.1 | MYB84 | 360 |
| | SbiRTX430.04G340300.v2.1 | MYB84 | 361 |
| | SbiRTX430.06G031600.v2.1 | MYB84 | 362 |
| | SbiRTX430.07G098600.v2.1 | MYB84 | 363 |
| | SbiRTX430.08G198200.v2.1 | MYB84 | 364 |
| | SbiRTX430.09G239100.v2.1 | MYB84 | 365 |
| | Sobic.001G075300.v3.2 | MYB84 | 366 |
| | Sobic.002G139800.v3.2 | MYB84 | 367 |
| | Sobic.002G209300.v3.2 | MYB84 | 368 |
| | Sobic.003G034500.v3.2 | MYB84 | 369 |
| | Sobic.003G260300.v3.2 | MYB84 | 370 |
| | Sobic.003G280000.v3.2 | MYB84 | 371 |
| | Sobic.004G322800.v3.2 | MYB84 | 372 |
| | Sobic.006G030200.v3.2 | MYB84 | 373 |
| | Sobic.007G092300.v3.2 | MYB84 | 374 |
| | Sobic.008G187001.v3.2 | MYB84 | 375 |
| | Sobic.009G224500.v3.2 | MYB84 | 376 |
| | Sobic.K042600.v3.2 | MYB84 | 377 |
| Thlaspi | Ta1014.HPIv04_4_004429 | ASFT | 378 |
| arvense | Ta1014.HPIv04_7_000300 | ASFT, FACT | 379 |
| | Ta1014.HPIv04_2_003931 | FACT | 380 |
| | Ta1014.HPIv04_1_002774 | GPAT5 | 381 |
| | Ta1014.HPIv04_6_003710 | GPAT5 | 382 |
| | Ta1014.HPIv04_1_000177 | HORST | 383 |
| | Ta1014.HPIv04_2_004054 | MYB84 | 384 |
| | Ta1014.HPIv04_4_002349 | MYB84 | 385 |
| | Ta1014.HPIv04_7_000368 | MYB84 | 386 |
| | Ta1014.HPIv04_1_002102 | RALPH | 387 |
| | Ta1014.HPIv04_2_003791 | RALPH | 388 |
| Zea mays | Zm00001d011979.RefGen_V4 | ASFT, FACT | 389 |
| | Zm00001d012274.RefGen_V4 | ASFT, FACT | 390 |
| | Zm00001d042723.RefGen_V4 | ASFT, FACT | 391 |
| | Zm00001d010520.RefGen_V4 | GPAT5 | 392 |
| | Zm00001d038366.RefGen_V4 | GPAT5 | 393 |
| | Zm00001d012326.RefGen_V4 | HORST | 394 |
| | Zm00001d042814.RefGen_V4 | HORST | 395 |
| | Zm00001d004126.RefGen_V4 | MYB84 | 396 |
| | Zm00001d005307.RefGen_V4 | MYB84 | 397 |
| | Zm00001d005784.RefGen_V4 | MYB84 | 398 |
| | Zm00001d011614.RefGen_V4 | MYB84 | 399 |
| | Zm00001d011739.RefGen_V4 | MYB84 | 400 |
| | Zm00001d013164.RefGen_V4 | MYB84 | 401 |
| | Zm00001d018319.RefGen_V4 | MYB84 | 402 |
| | Zm00001d018320.RefGen_V4 | MYB84 | 403 |
| | Zm00001d020569.RefGen_V4 | MYB84 | 404 |
| | Zm00001d034207.RefGen_V4 | MYB84 | 405 |
| | Zm00001d038930.RefGen_V4 | MYB84 | 406 |
| | Zm00001d039496.RefGen_V4 | MYB84 | 407 |
| | Zm00001d043611.RefGen_V4 | MYB84 | 408 |
| | Zm00001d043837.RefGen_V4 | MYB84 | 409 |
| | Zm00001d050185.RefGen_V4 | MYB84 | 410 |
| | Zm00001d052069.RefGen_V4 | MYB84 | 411 |

The present disclosure provides an isolated nucleic acid molecule comprising an isolated nucleic acid sequence encoding a protein of interest (such as MYB41), which is operably linked to a nucleic acid sequence encoding a heterologous promoter selected from the group comprising SEQ ID NO: 1, SEQ ID NO: 2, SEQ ID NO: 3, SEQ ID NO: 4, SEQ ID NO: 5 and SEQ ID NO: 6. In some examples, the heterologous promoter is a native promoter of FACT gene. In some examples, the heterologous promoter is a native promoter of HORST gene. In some examples, the heterologous promoter is a native promoter of ASFT gene. In some examples, the heterologous promoter is a native promoter of GPAT5 gene. In some examples, the heterologous promoter is a native promoter of RALPH gene. In some examples, the heterologous promoter is a native promoter of MYB84 gene.

In further examples, the native promoters of FACT gene, HORST gene, ASFT gene, GPAT5 gene, RALPH gene, and/or MYB84 gene can be derived, obtained, isolated from various plants (including monocots, dicots, vascular plants reproduced from spores).

In further examples, promoters of FACT gene, HORST gene, ASFT gene, GPAT5 gene, RALPH gene, and/or MYB84 derived, obtained, isolated from Arabidopsis and other plants taught herein (including monocots, dicots, vascular plants reproduced from spores) are operably linked to a target gene or a transgene of interest (such as wild-type MYB41 gene, homologs of MYB41, orthologs of MYB41 and/or paralogs of MYB41, and/or fragments and variations thereof) for increased suberin levels by altering gene expression patterns in a cell-type specific manner.

In some examples, a chimeric gene comprises an isolated nucleic acid sequence described above, which is operably linked to suitable regulatory sequences including, but not limited to native promoters of FACT orthologs, HORST orthologs, ASFT orthologs, GPAT5 orthologs, RALPH orthologs and/or MYB84 orthologs.

The present disclosure provides that the heterologous promoter operably linked to a nucleic acid sequence set forth in one of SEQ ID NOs: 92-201 can include an isolated nucleic acid sequence set forth in any one of SEQ ID NO: 1, SEQ ID NO: 2, SEQ ID NO: 3, SEQ ID NO: 4, SEQ ID NO: 5 and SEQ ID NO: 6, or a sequence at least about 50%, at least about 55%, at least about 60%, at least about 65%, at least about 70%, at least about 75%, at least about 80%, at least about 85%, at least about 90%, at least about 95%, at least about 98% identical thereto.

The heterologous promoter operably linked to a nucleic acid sequence, such as one of SEQ ID NOs: 92-201, can include an isolated nucleic acid sequence set forth in any one or more of SEQ ID NOs: 233-411, or a sequence at least about 50%, at least about 55%, at least about 60%, at least about 65%, at least about 70%, at least about 75%, at least about 80%, at least about 85%, at least about 90%, at least about 95%, at least about 98% identical thereto.

The heterologous promoter operably linked to a nucleic acid sequence, such as one of SEQ ID NOs: 92-201, can include at least 1, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, at least 1300, at least 1350, at least 1400, at least 1450, at least 1500, at least 1550, at least 1600, at least 1650, at least 1700, at least 1750, at least 1800, at least 1850, at least 1900, or at least 1950 consecutive nucleotides of any one or more of SEQ ID NOs: 1-6 and/or 233-411. In some examples, the promoter is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identical to the consecutive nucleotides of the one or more of SEQ ID NOs: 1-6 and/or 233-411 described above.

The heterologous promoter operably linked to a nucleic acid sequence, such as one of SEQ ID NOs: 92-201, can include at most 5, at most 10, at most 20, at most 30, at most 40, at most 50, at most 60, at most 70, at most 80, at most 90, at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1050, at most 1100, at most 1150, at most 1200, at most 1250, at most 1300, at most 1350, at most 1400, at most 1450, at most 1500, at most 1550, at most 1600, at most 1650, at most 1700, at most 1750, at most 1800, at most 1850, at most 1900, or at most 1950, or at most 2000 consecutive nucleotides of any one or more of SEQ ID NOs: 1-6 and/or 233-411. In some examples, the promoter is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identical to the consecutive nucleotides of the one or more of SEQ ID NOs: 1-6 and/or 233-411 described above.

The heterologous promoter operably linked to a nucleic acid sequence, such as one of SEQ ID NOs: 92-201, can include about 5, about 10, about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, or about 1950, or about 2000 consecutive nucleotides of any one or more of SEQ ID NOs: 1-6 and/or 233-411, or a range between any two of the preceding values. In some examples, the promoter is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identical to the consecutive nucleotides of the one or more of SEQ ID NOs: 1-6 and/or 233-411 described above.

In some examples, the promoter can include at most 500, 400, 300, 200, 100, 50, 45, 40, 35, 30, 25, 20, 15, 14, 13, 12, 11, 10, 9, 8, 7, 6, 5, 4, 3, 2, or 1 nucleic acid substitutions in any one or more of SEQ ID NOs: 1-6 and/or 233-411 provided the promoter functions, such that a nucleic acid sequence, such as one of SEQ ID NOs: 92-201 operably linked to the promoter can be expressed when transferred into a host cell.

In some examples, the present disclosure provides that native promoters of FACT orthologs, HORST orthologs, ASFT orthologs, GPAT5 orthologs, RALPH orthologs and/or MYB84 orthologs are derived from canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

In some examples, cell-type specific promoters derived from plants taught in the present disclosure are utilized for expression of MYB41 orthologs in plants taught in the resent disclosure such as canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), and maize (Zea mays).

### VI. Constructs

Constructs are provided that include expression cassettes containing promoters listed in Table 6 operably-linked to a MYB41 gene. When these constructs are transformed into plants, they enable those transgenic plants to develop additional periderm layers at an earlier stage of root development and/or deposit more suberin in periderm cells without negatively impacting plant health. Exemplary constructs are provided in Table 6. SEQ ID NOs: 7-12 are the DNA sequences of the entire transformation constructs, each of which that includes the expression cassette (SEQ ID NOs: 16-21; i.e. MYB41 gene, 3′UTR and Intergenic region operably linked to each promoter set forth in SEQ ID NOs: 1-6, respectively).

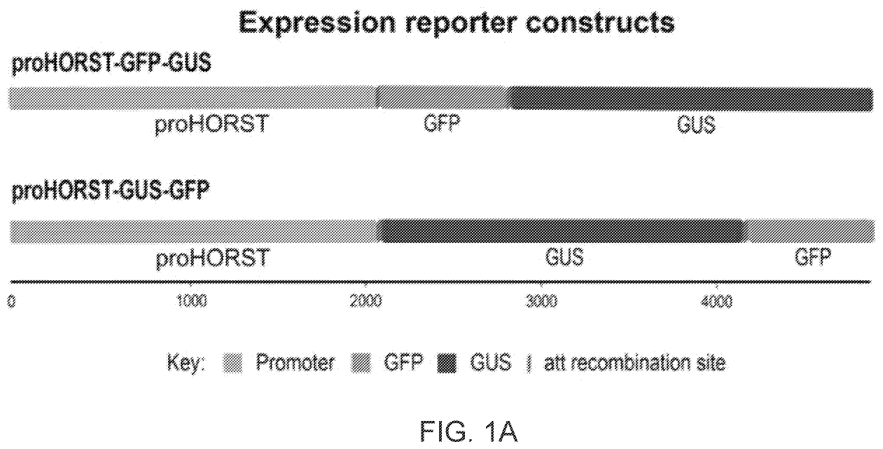

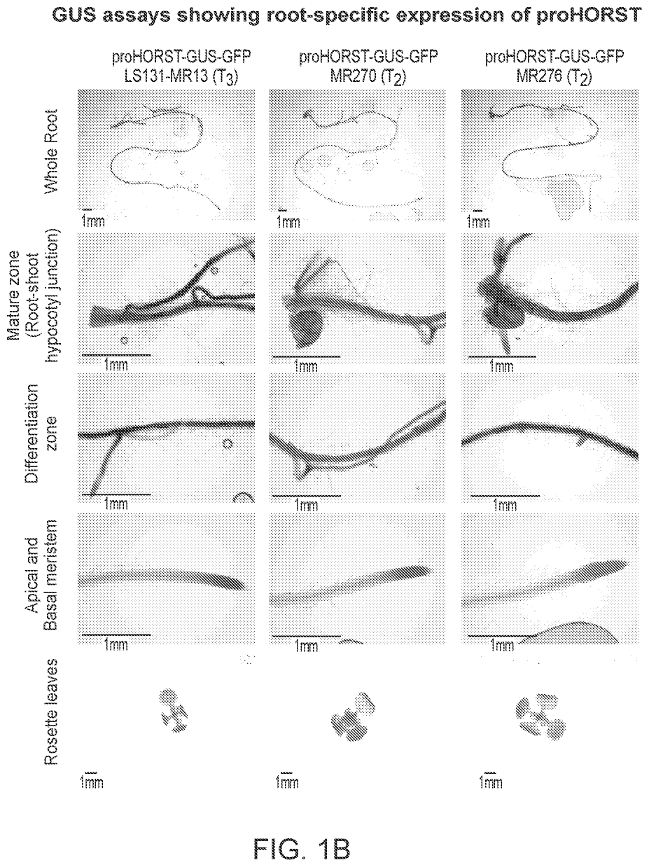

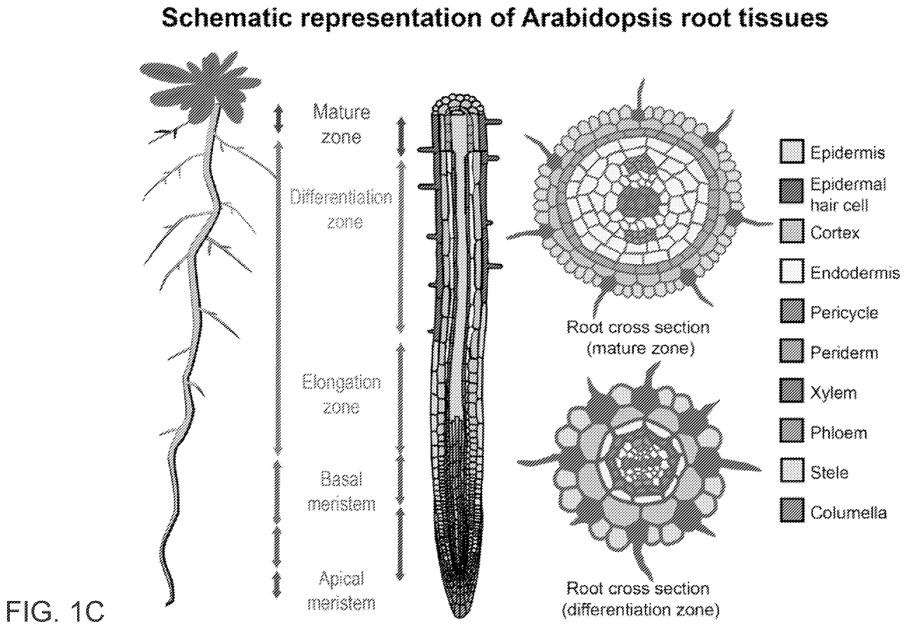

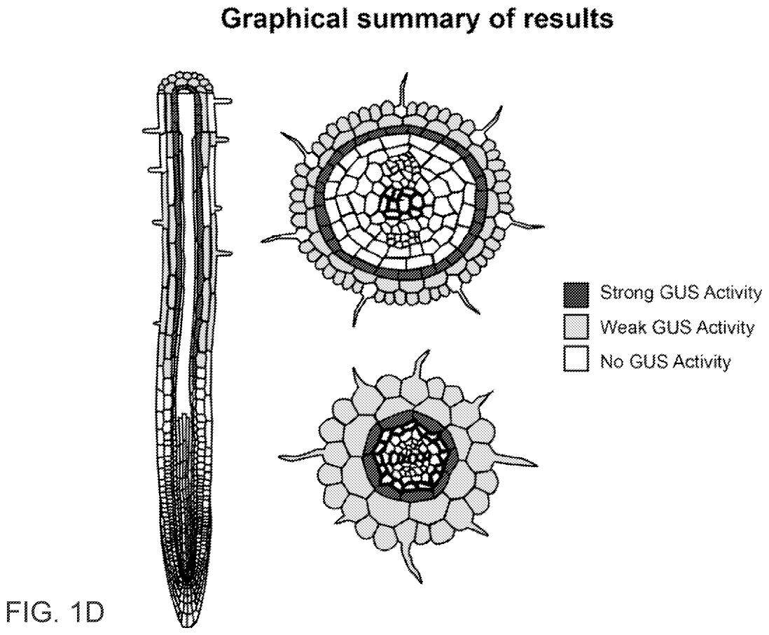

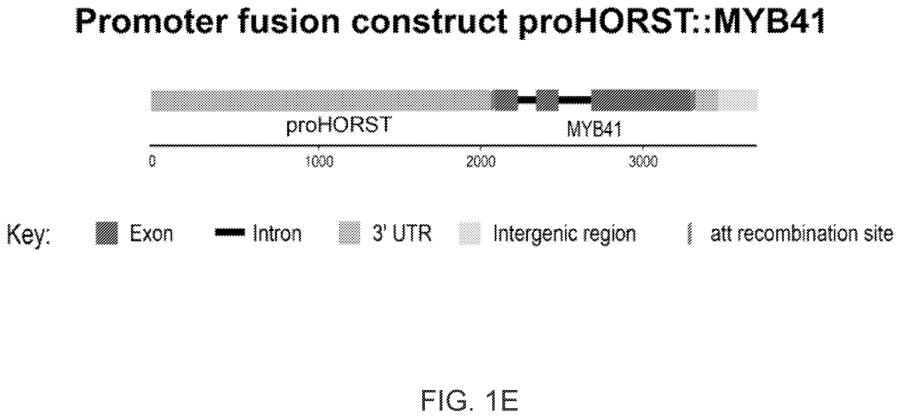

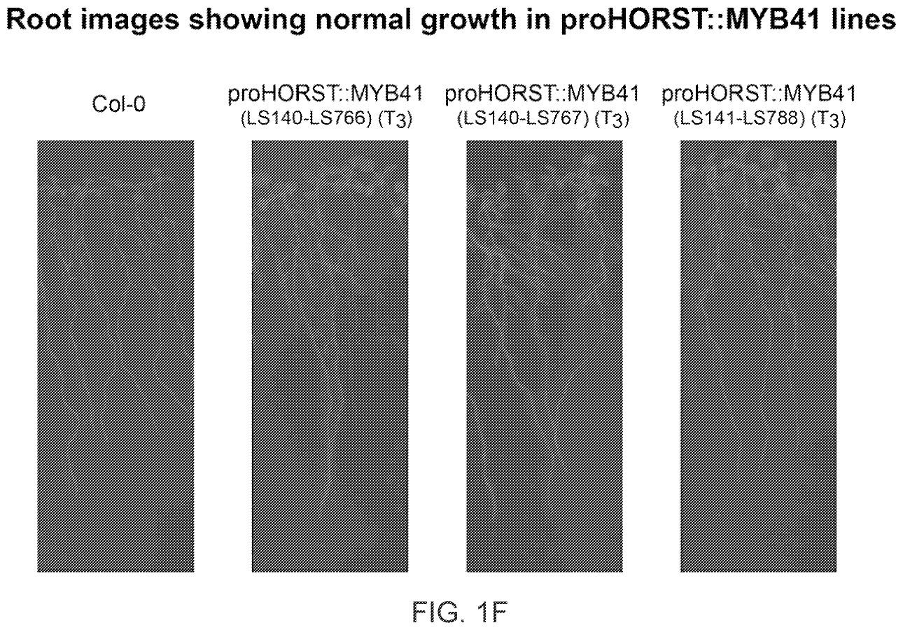

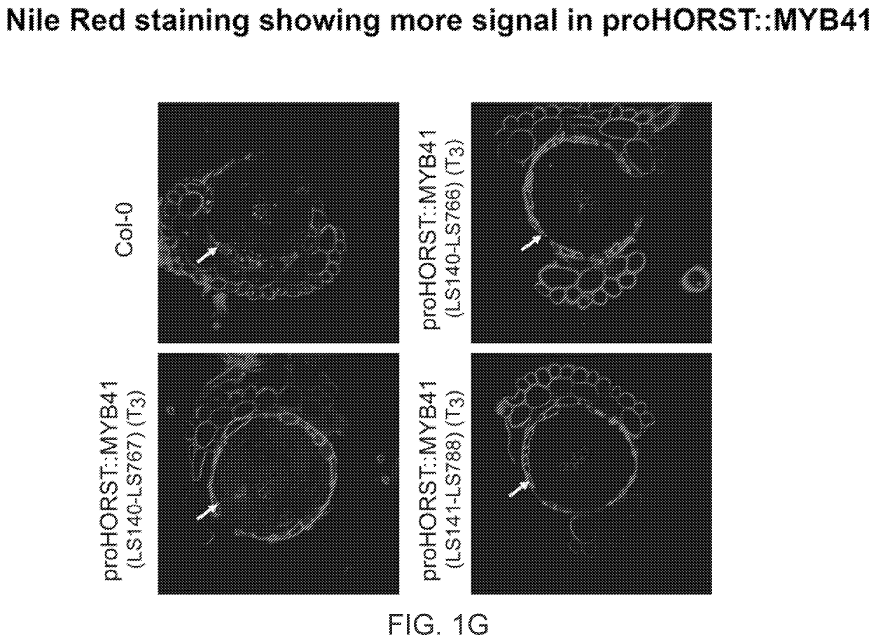

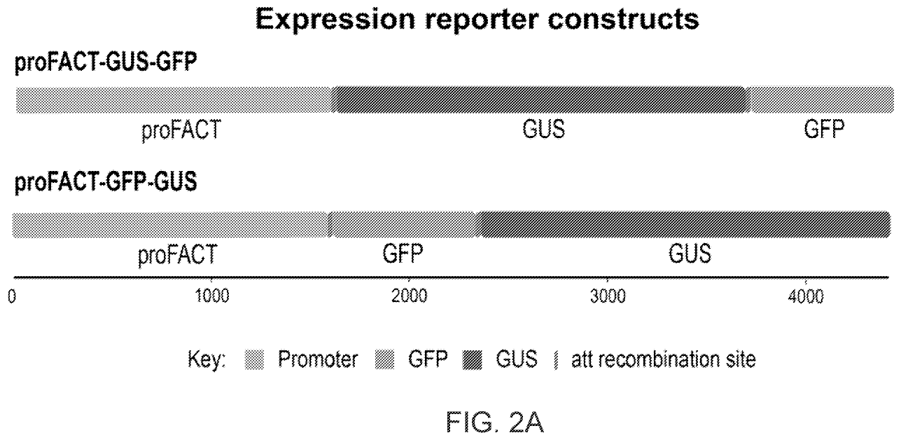

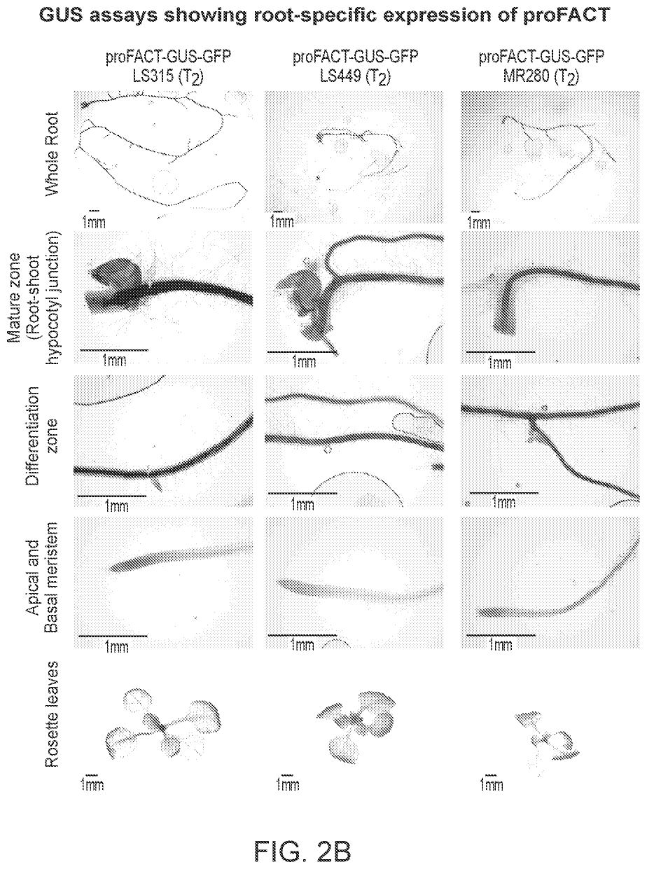

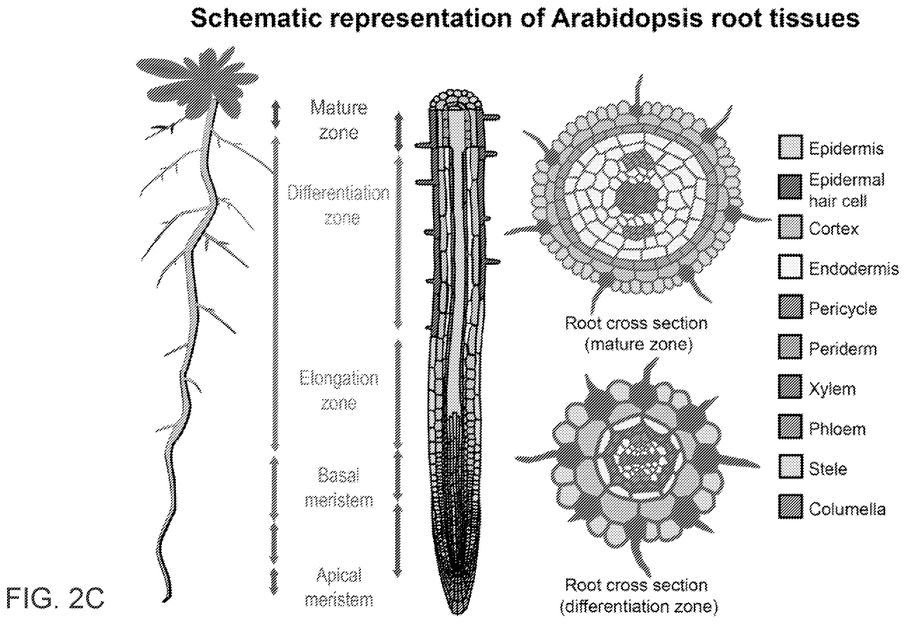

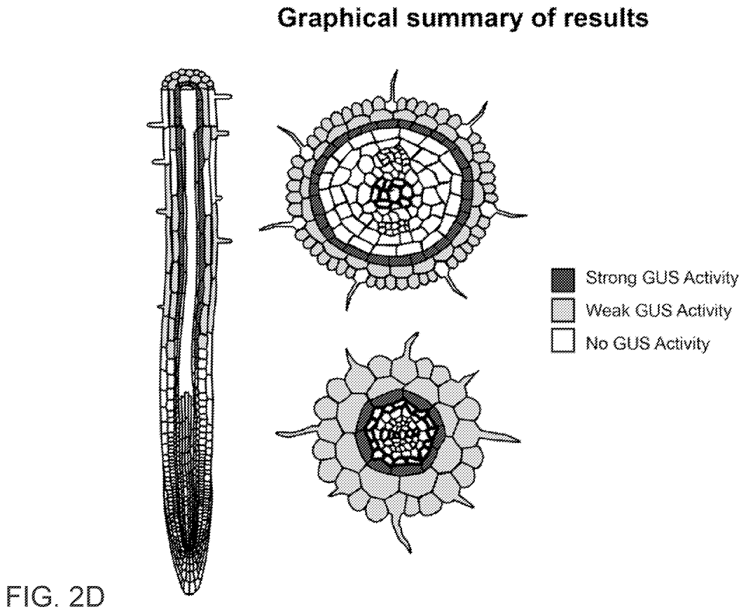

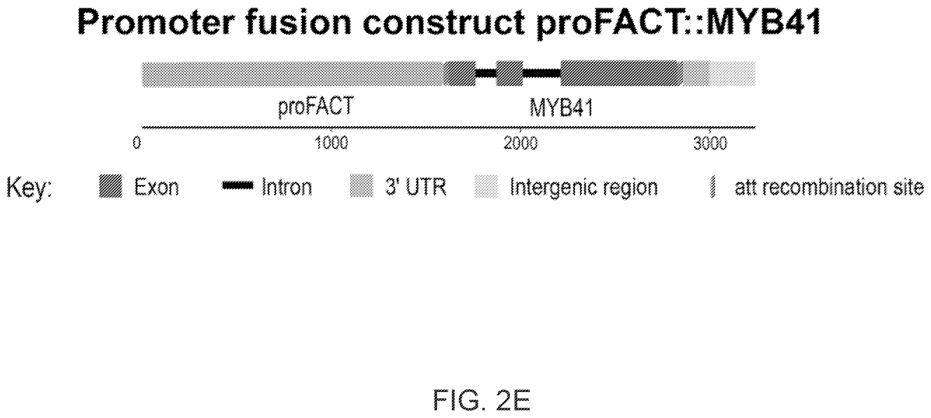

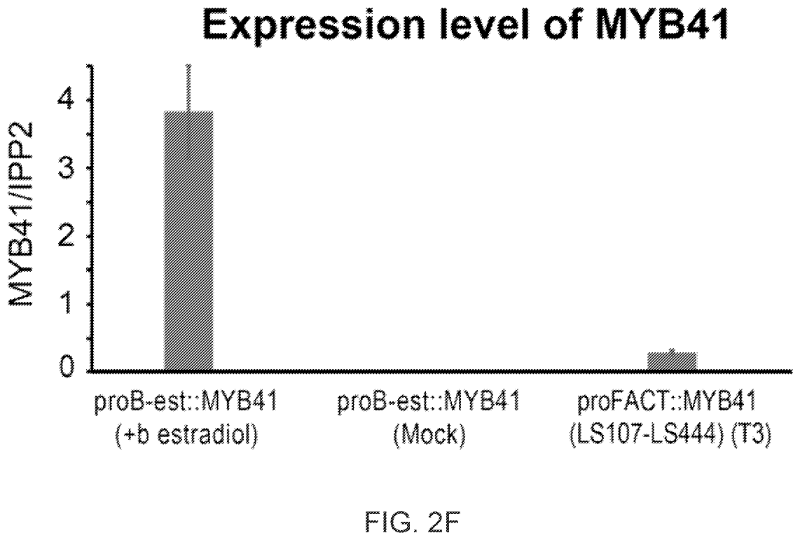

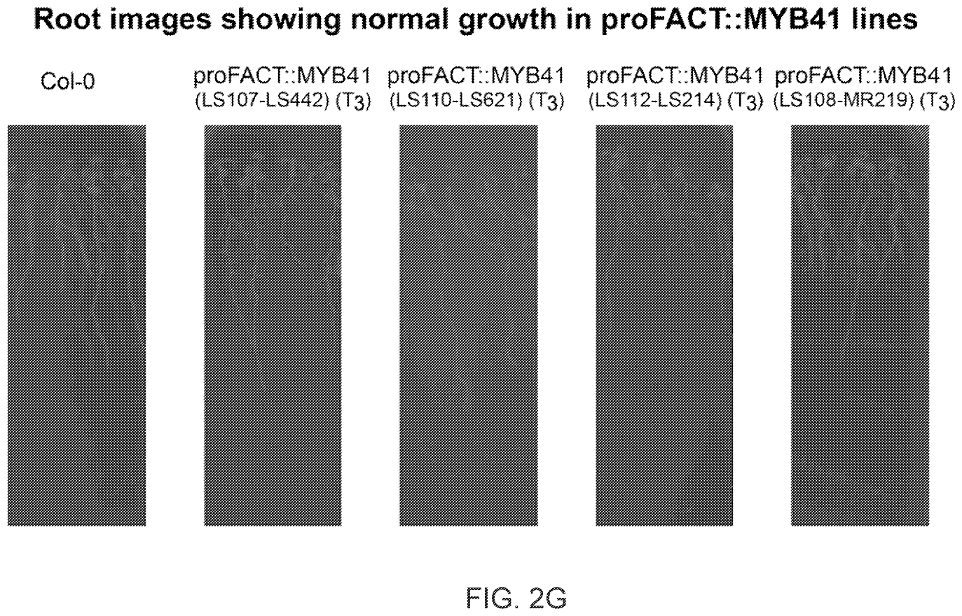

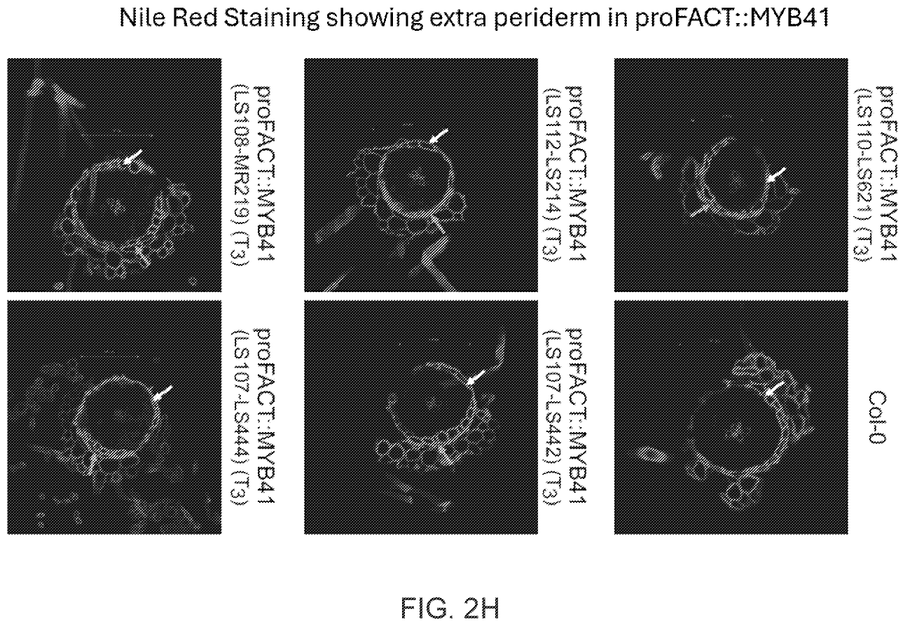

TABLE 6
Exemplary Transformation Construct and Expression
Cassette Sequences Driving MYB41 Expression

| | Nucleic Acid Sequence |
|---|---|
| Transformation Construct | |
| proFACT::AtMYB41 | SEQ ID NO: 7 |
| proHORST::AtMYB41 | SEQ ID NO: 8 |
| proASFT::AtMYB41 | SEQ ID NO: 9 |
| proGPAT5::AtMYB41 | SEQ ID NO: 10 |
| proRALPH::AtMYB41 | SEQ ID NO: 11 |
| proMYB84::AtMYB41 | SEQ ID NO: 12 |
| Expression Cassette | |
| proFACT::AtMYB41 (FIG. 2E) | SEQ ID NO: 16 |
| proHORST::AtMYB41 (FIG. 1E) | SEQ ID NO: 17 |
| proASFT::AtMYB41 | SEQ ID NO: 18 |
| proGPAT5::AtMYB41 | SEQ ID NO: 19 |
| proRALPH::AtMYB41 | SEQ ID NO: 20 |
| proMYB84::AtYB41 | SEQ ID NO: 21 |

The present disclosure also provides a recombinant construct comprising the chimeric gene as described above. In one example, said recombinant construct is a gene silencing construct, such as used in RNAi gene silencing. In another example, said recombinant construct is a gene editing construct, such as used in CRISPR-Cas gene editing system.

The expression vectors of the present disclosure may include at least one selectable marker. Such markers include dihydrofolate reductase, G418 or neomycin resistance for eukaryotic cell culture and tetracycline, kanamycin or ampicillin resistance genes for culturing in E. coli and other bacteria.

### VII. Plant Transformation

The provided polynucleotides coding for MYB41, homologs of MYB41, orthologs of MYB41 and/or paralogs of MYB41, and/or fragments and variations thereof can be transformed into plant cells, plant tissues, plant parts and whole plants.

Transgenic plants can be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

Agrobacterium tumefaciens is a naturally occurring bacterium capable of inserting its DNA (genetic information) into plants, resulting in a type of injury to the plant known as crown gall. Most species of plants can now be transformed using this method, including cucurbitaceous species.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues to get the plant cells to then express the new genes. The gene gun uses an explosive (.22 caliber blank) to propel the material. Compressed air or steam may also be used as the propellant. Most species of plants have been transformed using this method.

The most common method for the introduction of new genetic material into a plant genome involves the use of living cells of the bacterial pathogen Agrobacterium tumefaciens to inject a piece of DNA, called transfer or T-DNA, into individual plant cells (usually following wounding of the tissue) where it is targeted to the plant nucleus for chromosomal integration. There are numerous patents governing Agrobacterium-mediated transformation and particular DNA delivery plasmids designed specifically for use with Agrobacterium - - - for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Agrobacterium-mediated plant transformation involves placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA can also have applicability in the present disclosure. See, for example, U.S. Pat. No. 7,250,554, which utilizes P-DNA instead of T-DNA in the transformation vector.

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

Direct plant transformation methods using DNA have also been reported. The first of these to be reported historically is electroporation, which utilizes an electrical current applied to a solution containing plant cells (Fromm et al., Nature, 319, 791 (1986); Jones et al., Plant Mol. Biol., 13, 501 (1989) and Yang et al., Plant Cell Reports, 7, 421 (1988). Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, 5,015,580). A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed.

For efficient plant transformation, a selection method is typically employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of non-transformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19:259-268 (1982); Bevan et al., Nature 304:184-187 (1983)). However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl Acids Res 18:1062 (1990), Spencer et al., Theor Appl Genet 79:625-631 (1990), U.S. Pat. Nos. 4,795,855, 5,378,824 and 6,107,549). In addition, the dhfr gene, which confers resistance to the anticancer agent methotrexate, has been used for selection (Bourouis et al., EMBO J. 2 (7): 1099-1104 (1983).

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make expression units for use in the present disclosure. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Plant promoters such as prolifera promoter, fruit specific promoters, Ap3 promoter, heat shock promoters, seed specific promoters, etc. can also be used.

Either a gamete-specific promoter, a constitutive promoter (such as the CaMV or Nos promoter), an organ-specific promoter (such as the E8 promoter from tomato), or an inducible promoter is typically ligated to the protein or antisense encoding region using standard techniques. The expression unit may be further optimized by employing supplemental elements such as transcription terminators and/or enhancer elements.

Thus, for expression in plants, the expression units typically contains, in addition to the protein sequence, a plant promoter region, a transcription initiation site and a transcription termination sequence. Unique restriction enzyme sites at the 5′ and 3′ ends of the expression unit are typically included to allow for easy insertion into a pre-existing vector.

In the construction of heterologous promoter/structural gene or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. Polyadenylation sequences include, but are not limited to, the Agrobacterium octopine synthase signal (Gielen et al., EMBO J 3:835-846 (1984)) or the nopaline synthase signal (Depicker et al., Mol. and Appl. Genet. 1:561-573 (1982)). The resulting expression unit is ligated into or otherwise constructed to be included in a vector that is appropriate for higher plant transformation. One or more expression units may be included in the same vector. The vector will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, are generally also included to allow the vector to be cloned in a bacterial or phage host; preferably a broad host range for prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector, as is known in the art. For instance, in the case of Agrobacterium transformations, T-DNA sequences will also be included for subsequent transfer to plant chromosomes.

To introduce a desired gene or set of genes by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as improved fatty acid composition, and to introduce these genes into already useful varieties of plants. Once the foreign genes have been introduced into a plant, that plant can then be used in imp plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

While reducing the present disclosure to practice, the inventor can construct an expression construct which includes nucleotide sequences encoding MYB41, homologs of MYB41, orthologs of MYB41 and/or paralogs of MYB41, and/or fragments and variations thereof. The expression construct of the present disclosure can be introduced into embryogenic callus of any plant genus or species and the resulting transformed cells can be regenerated into plants. The transgenic plants are expected to have expression of the MYB41 protein.

The phrase “embryogenic callus cell” used herein refers to an embryogenic cell contained in a cell mass produced in vitro.

Several approaches can be utilized to transform and co-express these polynucleotides in plant cells.

In one aspect, each of the above described polynucleotide sequences can be separately introduced into a plant cell by using three separate nucleic-acid constructs. In some examples, the three polynucleotide sequences can be co-introduced and co-expressed in the plant cell using a single nucleic acid construct. Such a construct can be designed with a single promoter sequences co-which can transcribe a polycistronic message including all three polynucleotide sequences. To enable co-translation of the three polypeptides encoded by the polycistronic message, the polynucleotide sequences can be inter-linked via an internal ribosome entry site (IRES) sequence which facilitates translation of polynucleotide sequences positioned downstream of the IRES sequence. In this case, a transcribed polycistronic RNA molecule encoding the three polypeptides described above will be translated from both the capped 5′ end and the two internal IRES sequences of the polycistronic RNA molecule to thereby produce in the cell all three polypeptides.

Alternatively, the polynucleotide segments encoding the plurality of polypeptides capable of conferring increased suberin content in plant cells, plant tissues, plant parts and whole plants can be translationally fused via a protease recognition site cleavable by a protease expressed by the cell to be transformed with the nucleic acid construct. In this case, a chimeric polypeptide translated will be cleaved by a cell-expressed protease to thereby generate the plurality of polypeptides.

In other examples, the methods utilize a nucleic acid construct which includes three promoter sequences each capable of directing transcription of a specific polynucleotide sequence of the polynucleotide sequences described above.

Suitable promoters which can be used with the nucleic acid of the present disclosure include constitutive, inducible, or tissue-specific promoters.

Suitable constitutive promoters include, for example, CaMV 35S promoter (Odell et al., Nature 313:810-812, 1985); maize Ubi 1 (Christensen et al., Plant Sol. Biol. 18:675-689, 1992); rice actin (McElroy et al., Plant Cell 2:163-171, 1990); pEMU (Last et al., Theor. Appl. Genet. 81:581-588, 1991); and Synthetic Super MAS (Ni et al., The Plant Journal 7:661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable inducible promoters can be pathogen-inducible promoters such as, for example, the alfalfa PR10 promoter (Coutos-Thevenot et al., Journal of Experimental Botany 52:901-910, 2001 and the promoters described by Marineau et al., Plant Mol. Biol. 9:335-342, 1987; Matton et al. Molecular Plant-Microbe Interactions 2:325-331, 1989; Somsisch et al., Proc. Natl. Acad. Sci. USA 83:2427-2430, 1986: Somsisch et al., Mol. Gen. Genet. 2:93-98, 1988; and Yang, Proc. Natl. Acad. Sci. USA 93:14972-14977, 1996.

Suitable tissue-specific promoters include, but not limited to, leaf-specific promoters such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993.

The nucleic acid constructs provided herein may also include at least one selectable marker such as, for example, nptII. In one aspect, the nucleic acid construct is a shuttle vector, which can propagate both in E. coli (wherein the construct comprises an appropriate selectable marker and origin of replication) and be compatible for propagation in cells. The construct according to the present disclosure can be, for example, a plasmid, a bacmid, a phagemid, a cosmid, a phage, a virus or an artificial chromosome, preferably a plasmid.

The nucleic acid constructs provided herein can be utilized to stably transform plant cells. The principle methods of causing stable integration of exogenous DNA into plant genome include two main approaches:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell and Vasil Academic Publishers, San Diego, Calif. (1989) p. 2-25; Gatenby, in Plant Biotechnology, eds. Kung and Arntzen and Butterworth Publishers, Boston, Mass. (1989) p. 93-112.
(ii) Direct DNA uptake: Paszkowski et al., in Cell Culture and Somatic Cell Genetics of Plants, Vol. 6, Molecular Biology of Plant Nuclear Genes eds. Schell and Vasil, Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. Suitable Agrobacterium-mediated procedures for introducing exogenous DNA to plant cells is described by Dougale et al. (Journal of General Virology, 79:2301-2311, 1998) and in U.S. Pat. No. 6,395,962.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Alternatively, the nucleic acid construct of the present disclosure can be introduced into plant cells by a microprojectiles bombardment. In this technique, tungsten or gold particles coated with exogenous DNA are accelerated toward the target cells. Suitable plant transformation procedures by microprojectiles bombardment are described by Sagi et al. (Biotechnology 13:481-485, 1995) and by Dougale et al. (Journal of General Virology, 79:2301-2311, 1998). Preferably, the nucleic acid construct of the present disclosure is introduced into plant cells by a microprojectiles bombardment procedure as described in Example 4 herein below.

Following transformation, the transformed cells are micropropagated to provide a rapid, consistent reproduction of the transformed material.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Stable integration of exogenous DNA sequence in the genome of the transformed plants can be determined using standard molecular biology techniques well known in the art such as PCR and Southern blot hybridization.

Although stable transformation is presently preferred, transient transformation of cultured cells, leaf cells, meristematic cells or the whole plant is also envisaged by the present disclosure.

Transient transformation can be effected by any of the direct DNA transfer methods described above or by viral infection using modified plant viruses.

Viral infection is preferred since is enables circumventing micropropagation and regeneration of a whole plant from cultured cells. Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman et al. (Communications in Molecular Biology: Viral Vectors, Cold Spring Harbor Laboratory, New York, pp. 172-189, 1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson et al. (Virology 172:285-292, 1989; Takamatsu et al. EMBO J. 6:307-311, 1987; French et al. (Science 231:1294-1297, 1986); and Takamatsu et al. (FEBS Letters 269:73-76, 1990).

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA.

If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present disclosure is demonstrated by the above references as well as in U.S. Pat. No. 5,316,931.

In one example, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second example, a recombinant plant viral nucleic acid is provided as in the first example except that the native coat protein coding sequence is placed adjacent one of the non-native coat protein subgenomic promoters instead of a non-native coat protein coding sequence.

In a third example, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth example, a recombinant plant viral nucleic acid is provided as in the third example except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present disclosure can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily affected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

### VIII. Breeding Methods

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, can depend upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. Comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988). For population improvement methods specific for soybean see, e.g., J. R. Wilcox, editor (1987) SOYBEANS: Improvement, Production, and Uses, Second Edition, American Society of Agronomy, Inc., Crop Science Society of America, Inc., and Soil Science Society of America, Inc., publishers, 888 pages.

Mass Selection. In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enters a synthetic varies widely. In practice, numbers of parental lines range from 10 to several hundred, with 100-200 being the average. Broad based synthetics formed from 100 or more clones would be expected to be more stable during seed multiplication than narrow based synthetics.

Hybrids. As discussed above, hybrid is an individual plant resulting from a cross between parents of differing genotypes. Commercial hybrids are now used extensively in many crops, including corn (maize), sorghum, sugar beet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids involves the isolated production of both the parental lines and the hybrids which result from crossing those lines, see, e.g., Wright, Commercial Hybrid Seed Production 8:161-176, In Hybridization of Crop Plants.

Bulk Segregation Analysis (BSA). BSA, a.k.a. bulked segregation analysis, or bulk segregant analysis, is described by Michelmore et al. (Michelmore et al., 1991, Identification of markers linked to disease-resistance genes by bulked segregant analysis: a rapid method to detect markers in specific genomic regions by using segregating populations. Proceedings of the National Academy of Sciences, USA, 99:9828-9832) and Quarrie et al. (Quarrie et al., Bulk segregant analysis with molecular markers and its use for improving drought resistance in maize, 1999, Journal of Experimental Botany, 50 (337): 1299-1306).

For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., resistant to pathogen), and the other from the individuals having reversed phenotype (e.g., susceptible to pathogen), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping.

Gene Pyramiding. The method to combine into a single genotype a series of target genes identified in different parents is usually referred as gene pyramiding. The first part of a gene pyramiding breeding is called a pedigree and is aimed at cumulating one copy of all target genes in a single genotype (called root genotype). The second part is called the fixation steps and is aimed at fixing the target genes into a homozygous state, that is, to derive the ideal genotype (ideotype) from the root genotype. Gene pyramiding can be combined with marker assisted selection (MAS, see Hospital et al., 1992, 1997a, and 1997b, and Moreau et al, 1998) or marker based recurrent selection (MBRS, see Hospital et al., 2000).

### IX. Gene Editing

A “gene editing system” includes one or more DNA-binding domains or components and one or more DNA-modifying domains or components, or isolated nucleic acids, e.g., one or more vectors, encoding said DNA-binding and DNA-modifying domains or components. Gene editing systems are used for modifying the nucleic acid of a target gene and/or for modulating the expression of a target gene. In known gene editing systems, for example, the one or more DNA-binding domains or components are associated with the one or more DNA-modifying domains or components, such that the one or more DNA-binding domains target the one or more DNA-modifying domains or components to a specific nucleic acid site. Methods and compositions for enhancing gene editing is well known in the art. See example, U.S. Patent Application Publication No. 2018/0245065, which is incorporated by reference in its entirety.

Exemplary gene editing systems that can be used include but are not limited to, zinc finger nucleases, transcription activator-like effector nucleases (TALENs); clustered regularly interspaced short palindromic repeats (CRISPR)/Cas systems, meganuclease systems, and viral vector-mediated gene editing.

In some examples, the present disclosure provides methods for gene editing/cloning utilizing DNA nucleases. CRISPR complexes, transcription activator-like effector nucleases (TALENs), zinc finger nucleases (ZFNs), and FokI restriction enzymes. These enzymes target their nuclease activities to desired target loci through interactions with guide regions engineered to recognize sequences of interest. In some examples, the present disclosure provides CRISPR-based gene editing methods to genetically engineer the genome of plant species of the present disclosure in order to stimulate, enhance, or modulate suberin content of plant cells, plant tissues, plant parts or whole plants.

### (i) CRISPR Systems

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge bv integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al., Nat. Struct. Mol. Biol. 2001, 18:688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in Class 2 systems all functions of the effector complex are carried out by a single protein (e.g., Cas9 or Cpf1). In some examples, the present disclosure provides using type II and/or type V single-subunit effector systems.

As these naturally occur in many different types of bacteria, the exact arrangements of the CRISPR and structure, function and number of Cas genes and their product differ somewhat from species to species. Haft et al. (2005) PLOS Comput. Biol. 1: e60; Kunin et al. (2007) Genome Biol. 8: R61; Mojica et al. (2005) J. Mol. Evol. 60:174-182; Bolotin et al. (2005) Microbiol. 151:2551-2561; Pourcel et al. (2005) Microbiol. 151:653-663; and Stern et al. (2010) Trends. Genet. 28:335-340. For example, the Cse (Cas subtype, E. coli) proteins (e.g., CasA) form a functional complex, Cascade, which processes CRISPR RNA transcripts into spacer-repeat units that Cascade retains. Brouns et al. (2008) Science 321:960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341:833-836.

### (ii) CRISPR/Cas9

In some examples, the present disclosure provides methods of gene editing using a Type II CRISPR system. Type II systems rely on a i) single endonuclease protein, ii) a transactiving crRNA (tracrRNA), and iii) a crRNA where a ~20-nucleotide (nt) portion of the 5′ end of crRNA is complementary to a target nucleic acid. The region of a CRISPR crRNA strand that is complementary to its target DNA protospacer is hereby referred to as “guide sequence.”

In some examples, the tracrRNA and crRNA components of a Type II system can be replaced by a single guide RNA (sgRNA), also known as a guide RNA (gRNA). The sgRNA can include, for example, a nucleotide sequence that comprises an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks, and are recruited to target DNA by a combination of a crRNA and a tracrRNA oligos, which tether the endonuclease via complementary hybridization of the RNA CRISPR complex.

In some examples, DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., et. al., Science. 2012, 337:816-821). In some examples, the PAM motif recognized by a Cas9 varies for different Cas9 proteins.

In some examples a Cas9 is any variant derived or isolated from any source. In other examples, the Cas9 peptide includes one or more of the mutations described in the literature, including but not limited to the functional mutations described in: Fonfara et al. Nucleic Acids Res. 2014 February; 42 (4): 2577-90; Nishimasu H. et al. Cell. 2014 Feb. 27,156 (5): 935-49; Jinek M. et al. Science. 2012 337:816-21; and Jinek M. et al. Science. 2014 Mar. 14, 343 (6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some examples, the systems and methods disclosed herein can be used with the wild type Cas9 protein having double-stranded nuclease activity, Cas9 mutants that act as single stranded nickases, or other mutants with modified nuclease activity.

According to the present disclosure, Cas9 molecules of, derived from, or based on the Cas9 proteins of a variety of species can be used in the methods and compositions described herein. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, Gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some examples, the present disclosure provides the use of tools for genome editing techniques in plants such as crops and methods of gene editing using CRISPR-associated (cas) endonucleases including SpyCas9, SaCas9, St1Cas9. See example, Song et al. (2016), CRISPR/Cas9: A powerful tool for crop genome editing, The Crop Journal 4:75-82, Mali et al. (2013) RNA-guided human genome engineering via cas9, Science 339:823-826; Ran et al. (2015) In vivo genome editing using Staphylococcus aureus cas9, Nature 520:186-191; Esvelt et al. (2013) Orthogonal cas9 proteins for rna-guided gene regulation and editing, Nature methods 10 (11): 1116-1121, each of which is hereby incorporated by reference in its entirety for all purposes.

(iii) CRISPR/Cpf1

In other examples, methods of gene editing use a Type V CRISPR system. In some examples, the present disclosure provides methods of gene editing using CRISPR from Prevotella, Francisella, Acidaminococcus, Lachnospiraceae, and Moraxella (Cpf1).

The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some examples, guide sequences for Cpf1 must be at least 12 nt, 13 nt, 14 nt, 15 nt, or 16 nt to achieve detectable DNA cleavage, and a minimum of 14 nt, 15 nt, 16 nt, 17 nt, or 18 nt to achieve efficient DNA cleavage.

Cpf1 systems differ from Cas9 in a variety of ways. First, unlike Cas9, Cpf1 does not require a separate tracrRNA for cleavage. In some examples, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long.

Second, certain Cpf1 systems prefer a “TTN” PAM motif that is located 5′ upstream of its target. This is in contrast to the “NGG” PAM motifs located on the 3′ of the target DNA for common Cas9 systems such as Streptococcus pyogenes Cas9. In some examples, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with 3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on Zetsche B. et al. 2015. (“Cpf1 Is a Single RNA-Guided Endonuclease of a Class 2 CRISPR-Cas System” Cell 163, 759-771).

(iv) Guide RNA (gRNA)

In some examples, the guide RNA of the present disclosure includes two coding regions, encoding for crRNA and tracrRNA, respectively. In other examples, the guide RNA is a single guide RNA (sgRNA) synthetic crRNA/tracrRNA hybrid. In other examples, the guide RNA is a crRNA for a Cpf1 endonuclease.

Unless otherwise noted, all references to a single guide RNA (sgRNA) in the present disclosure can be read as referring to a guide RNA (gRNA). Therefore, examples described in the present disclosure which refer to a single guide RNA (sgRNA) will also be understood to refer to a guide RNA (gRNA).

The guide RNA is designed to recruit the CRISPR endonuclease to a target DNA region. In some examples, the present disclosure provides methods of identifying viable target CRISPR landing sites, and designing guide RNAs for targeting the sites. For example, in some examples, algorithms are designed to facilitate the identification of CRISPR landing sites within target DNA regions.

In some examples, software programs are designed to identify candidate CRISPR target sequences on both strands of an input DNA sequence based on desired guide sequence length and a CRISPR motif sequence (PAM, protospacer adjacent motif) for a specified CRISPR enzyme. For example, target sites for Cpf1 from Francisella novicida U112, with PAM sequences TTN, may be identified by searching for 5′-TTN-3′ both on the input sequence and on the reverse-complement of the input. The target sites for Cpf1 from Lachnospiraceae bacterium and Acidaminococcus sp., with PAM sequences TTTN, may be identified by searching for 5′-TTTN-3′ both on the input sequence and on the reverse complement of the input. Likewise, target sites for Cas9 of S. thermophilus CRISPR, with PAM sequence NNAGAAW, may be identified by searching for 5′-Nx-NNAGAAW-3′ both on the input sequence and on the reverse-complement of the input. The PAM sequence for Cas9 of S. pyogenes is 5′-NGG-3′.

Since multiple occurrences in the genome of the DNA target site may lead to nonspecific genome editing, after identifying all potential sites, sequences may be filtered out based on the number of times they appear in the relevant reference genome or modular CRISPR construct. For those CRISPR enzymes for which sequence specificity is determined by a ‘seed’ sequence (such as the first 5 bp of the guide sequence for Cpf1-mediated cleavage) the filtering step may also account for any seed sequence limitations.

In some examples, algorithmic tools can also identify potential off target sites for a particular guide sequence. For example, in some examples Cas-Offinder can be used to identify potential off target sites for Cpf1 (see Kim et al., 2016. “Genome-wide analysis reveals specificities of Cpf1 endonucleases in human cells” Nature Biotechnology 34, 863-868). Any other publicly available CRISPR design/identification tool may also be used, including for example the Zhang lab crispr.mit.edu tool (see Hsu, et al. 2013 “DNA targeting specificity of RNA guided Cas9 nucleases” Nature Biotech 31, 827-832).

In some examples, the user may be allowed to choose the length of the seed sequence. The user may also be allowed to specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s).

In the guide RNA, the “spacer/guide sequence” sequence is complementary to the “proto spacer” sequence in the DNA target. The gRNA “scaffold” for a single stranded gRNA structure is recognized by the Cas9 protein.

In some examples, the transgenic plant, plant part, plant cell, or plant tissue culture taught in the present disclosure comprise a recombinant construct, which comprises at least one nucleic acid sequence encoding a guide RNA. In some examples, the nucleic acid is operably linked to a promoter. In other examples, a recombinant construct further comprises a nucleic acid sequence encoding a Clustered regularly interspaced short palindromic repeats (CRISPR) endonuclease. In other examples, the guide RNA is capable of forming a complex with said CRISPR endonuclease, and said complex is capable of binding to and creating a double strand break in a genomic target sequence of said plant genome. In other examples, the CRISPR endonuclease is Cas9.

New breeding techniques (NBTs) refer to various new technologies developed and/or used to create new characteristics in plants through genetic variation, the aim being targeted mutagenesis, targeted introduction of new genes or gene silencing (RdDM). Exemplary NBTs include: targeted sequence changes facilitated through the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM, a.k.a., site-directed mutagenesis), Cisgenesis and intragenesis, epigenetic approaches such as RNA-dependent DNA methylation (RdDM, which does not necessarily change nucleotide sequence but can change the biological activity of the sequence), Grafting (on GM rootstock), Reverse breeding, Agro-infiltration for transient gene expression (agro-infiltration “sensu stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease, re-engineered homing endonucleases, DNA guided genome editing (Gao et al., Nature Biotechnology (2016), doi: 10.1038/nbt.3547, incorporated by reference in its entirety), and Synthetic genomics. A major part of today's targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques-State-of-the-art and prospects for commercial development”, which is incorporated by reference in its entirety.

In further examples, the target sequence is a nucleic acid for MYB41, homologs of MYB41, orthologs of MYB41 and/or paralogs of MYB41, and/or fragments and variations thereof. In some examples, the present disclosure provides the gene editing of MYB41 in plants using genetic engineering techniques described herein.

In some examples, the modified plant cells comprise one or more modifications (e.g., insertions, deletions, or mutations of one or more nucleic acids) in the genomic DNA sequence of an endogenous target gene resulting in the altered function the endogenous gene, thereby modulating, stimulating, or enhancing suberin content in plant cells, plant tissues, plant parts and whole plants. In such examples, the modified plant cells comprise a “modified endogenous target gene.” In some examples, the modifications in the genomic DNA sequence cause mutation, thereby altering the function of the MYB41 protein. In some examples, the modifications in the genomic DNA sequence results in amino acid substitutions, thereby altering the normal function of the encoded protein. In some examples, the modifications in the genomic DNA sequence encode a modified endogenous protein with modulated, altered, stimulated or enhanced function compared to the unmodified version of the endogenous protein.

In some examples, the modified plant cells described herein comprise one or more modified endogenous target genes, wherein the one or more modifications result in an altered function of a gene product (i.e., a protein) encoded by the endogenous target gene compared to an unmodified plant cell. For example, in some examples, a modified plant cell demonstrates expression of a protein or an upregulated expression of said protein. In some examples, the expression of the gene product (such as genetically-engineered MYB41) in a modified plant cell is enhanced by at least 0.5%, 1%, 2%, 3%, 4%, 5% or higher compared to the expression of the gene product (such as MYB41) in an unmodified plant cell. In other examples, the expression of the gene product (such as genetically-engineered MYB41) in a modified plant cell is enhanced by at least 10%, 20%, 30%, 40%, 50%, 60%, 70%, 80%, 90%, or more compared to the expression of the gene product (such as MYB41) in an unmodified plant cell. In some examples, the modified plant cells described herein demonstrate enhanced expression and/or function of gene products encoded by a plurality (e.g., two or more) of endogenous target genes compared to the expression of the gene products in an unmodified plant cell. For example, in some examples, a modified plant cell demonstrates enhanced expression and/or function of gene products from 2, 3, 4, 5, 6, 7, 8, 9, 10, or more endogenous target genes compared to the expression of the gene products in an unmodified plant cell.

In some examples, the modified plant cells described herein comprise one or more modified endogenous target genes, wherein the one or more modifications to the target DNA sequence results in expression of a protein with reduced or altered function (e.g., a “modified endogenous protein”) compared to the function of the corresponding protein expressed in an unmodified plant cell (e.g., a “unmodified endogenous protein”). In some examples, the modified plant cells described herein comprise 2, 3, 4, 5, 6, 7, 8, 9, 10, or more modified endogenous target genes encoding 2, 3, 4, 5, 6, 7, 8, 9, 10, or more modified endogenous proteins. In some examples, the modified endogenous protein demonstrates enhanced or altered binding affinity for another protein expressed by the modified plant cell or expressed by another cell; enhanced or altered signaling capacity; enhanced or altered enzymatic activity; enhanced or altered DNA-binding activity; or reduced or altered ability to function as a scaffolding protein.

The present disclosure also provides a transformed host cell comprising the chimeric gene as described above. In one example, said host cell is selected from the group consisting of bacteria, yeasts, filamentous fungi, algae, animals, and plants.

These sequences allow the design of gene-specific primers and probes for MYB41, homologs of MYB41, orthologs of MYB41, homeologs of MYB41, paralogs of MYB41, and fragments and variations thereof.

### X. Plants for Use with the Disclosed Methods

The present disclosure describes the plant to be transformed with a plant transformation construct or vector taught herein. The methods for targeted gene-editing system as described herein can be used to confer desired traits on essentially any plant. A wide variety of plants and plant cell systems may be engineered for the desired physiological and agronomic characteristics described herein using the nucleic acid constructs of the present disclosure and the various transformation methods. In some examples, the plant for the transformation is a monocotyledonous plant (monocot) or a dicotyledonous plant (dicot).

Monocots include a flowering plant having embryos with one cotyledon or seed leaf, parallel leaf veins, and flower parts in multiples of three. Examples of monocots used for transformation, genetic engineering or gene-editing include, but are not limited to turfgrass, corn/maize, rice, oat, annual ryegrass, wheat, barley, sorghum, orchid, iris, lily, onion, and palm. Examples of turfgrass include, but are not limited to Agrostis spp. (bentgrass species including colonial bentgrass and creeping bentgrasses), Poa pratensis (Kentucky bluegrass), Lolium spp. (ryegrass species including annual ryegrass and perennial ryegrass), Festuca arundinacea (tall fescue) Festuca rubra commutata (Chewings fescue), Cynodon dactylon (bermudagrass, Pennisetum clandestinum (kikuyu grass), Stenotaphrum secundatum (St. Augustine grass), Zoysia japonica (zoysia grass), and Dichondra micrantha.

Exemplary plants covered by the present disclosure include, without limitation, angiosperm and gymnosperm plants such as acacia, alfalfa, amaranth, apple, apricot, artichoke, ash tree, asparagus, avocado, banana, barley, beans, beet, birch, beech, blackberry, black raspberry, blueberry, broccoli, Brussel's sprouts, cabbage, cane berry, canola, cantaloupe, carrot, cassava, cauliflower, cedar, a cereal, celery, chestnut, cherry, Chinese cabbage, citrus, Clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, peach, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, wild strawberry, yams, yew, and zucchini.

In further examples, plants and plant cells for transformation, genetic engineering or gene-editing, for example to express MYB41 from a heterologous promoter, include, but are not limited to, those monocotyledonous and dicotyledonous plants, such as crops including grain crops (e.g., wheat, maize, rice, millet, barley), fruit crops (e.g., tomato, apple, grape, peach, pear, plum, raspberry, black raspberry, blackberry, cane berry, cherry, avocado, strawberry, wild strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). In some examples, fruit crops such as tomato, apple, peach, pear, plum, raspberry, black raspberry, blackberry, cane berry, cherry, avocado, strawberry, wild strawberry, grape and orange.

In further examples, plants and plant cells for transformation, genetic engineering or gene-editing, for example to express MYB41 from a heterologous promoter, include, but are not limited to, Canola (Brassica napus), Soybean (Glycine max), Cotton (Gossypium hirsutum), Rice (Oryza sativa), Lotus (Lotus japonicus), Radish (Raphanus sativus), Setaria (Setaria italica), Sorghum (Sorghum bicolor), Pennycress (Thlaspi arvense), Southern cattail (Typha domingensis), Wheat (Triticum aestivum), and Maize (Zea mays).

In some examples, the plant for transformation, genetic engineering or gene-editing, for example to express MYB41 from a heterologous promoter, is a dicot. In some examples, the plant, plant part, or plant cell is a species selected from Abrabidopsis genus, Brassica genus, Glycine genus, Gossypium genus, Oryza genus, Raphanus genus, Setaria genus, Sorghum genus, Thlaspi genus, Typha genus, Triticum genus, and Zea genus.

In examples, the plant, plant part, or plant cell is from the Abrabidopsis genus and is Abrabidopsis thaliana.

In examples, the plant, plant part, or plant cell is from the Brassica genus, such as Brassica balearica (Mallorca cabbage), Brassica carinata (Abyssinian mustard or Abyssinian cabbage), Brassica elongata (elongated mustard), Brassica fruticulosa (Mediterranean cabbage), Brassica hilarionis (St. Hilarion cabbage), Brassica juncea (Indian mustard, brown and leaf mustards, Sarepta mustard), Brassica napus (rapeseed, canola, rutabaga, Siberian kale), Brassica narinosa (broadbeaked mustard), Brassica nigra (black mustard), Brassica oleracea (kale, cabbage, collard greens, broccoli, cauliflower, kai-lan, brussels sprouts, kohlrabi), Brassica perviridis (tender green, mustard spinach), Brassica rapa (Chinese cabbage, turnip, rapini, komatsuna), Brassica rupestris, Brassica spinescens, and Brassica tournefortii (Asian mustard).

In examples, the plant, plant part, or plant cell is from the Thlaspi genus, such as Thlaspi alliaceum (roadside penny-cress), Thlaspi arcticum (arctic penny-cress), Thlaspi arvense (field penny-cress), Thlaspi caerulescens (alpine penny-cress), Thlaspi californicum (Kneeland Prairie penny-cress), Thlaspi cyprium (Cyprus penny-cress), Thlaspi fendleri (Fendler's penny-cress), Thlaspi idahoense (Idaho penny-cress), Thlaspi jankae (Slovak penny-cress), Thlaspi montanum (alpine penny-cress), Thlaspi parviflorum (meadow penny-cress), Thlaspi perfoliatum (Cotswold penny-cress), Thlaspi praecox (early penny-cress), or Thlaspi rotundifolium (round-leaved penny-cress).

The present disclosure provides a plant, plant part, or plant cell disclosed above, which is transformed with plant transformation vectors comprising: a nucleic acid encoding a MYB41 protein ortholog, such as an ortholog selected from SEQ ID NOs: 37-91; and ii) a promoter operably linked to the nucleic acid. In some examples, the promoter is a cell-type specific promoter. In some examples, the promoter is an Arabidopsis promoter. m some examples, the promoter is at least one of proFACT, proHORST, proASFT, proGPAT5, proRALPH, and/or proMYB84. In some examples, the promoter is at least one of SEQ ID NOs: 1, 2, 3, 4, 5, and/or 6. In some examples, the promoter a promoter of a FACT ortholog, a HORST ortholog, an ASFT ortholog, a GPAT5 ortholog, a RALPH ortholog and/or a MYB84 ortholog. In some examples, the promoter of a FACT ortholog includes at least one of SEQ ID NOs: 234, 239-246, 280-285, 314-316, 340-349, 379, 380, and/or 389-391. In some examples the promoter of a HORST ortholog includes at least one of SEQ ID NOs: 236, 254-258, 288, 289, 319-321, 352-354, 383, 394, and/or 395. In some examples the promoter of an ASFT ortholog includes at least one of SEQ ID NOs: 233, 239-244, 280-285, 314-316, 340-349, 378, 379, and/or 389-391. In some examples the promoter of a GPAT5 ortholog includes at least one of SEQ ID NOs: 235, 247-253, 286, 287, 317, 318, 350, 351, 381, 382, 392, and/or 393. In some examples the promoter of a RALPH ortholog includes at least one of SEQ ID NOs: 238, 387, and/or 388. In some examples the promoter of an MYB84 ortholog includes at least one of SEQ ID NOs: 237, 259-279, 290-313, 322-339, 355-377, 384-386, and/or 396-411. In some examples, the promoter is derived from Arabidopsis, canola (Brassica napus), soybean (Glycine max), cotton (Gossypium hirsutum), rice (Oryza sativa), Lotus (Lotus japonicus), Radish (Raphanus sativus), Setaria (Setaria italica), sorghum (Sorghum bicolor), pennycress (Thlaspi arvense), Southern cattail (Typha domingensis), wheat (Triticum aestivum), or maize (Zea mays). Exemplary Arabidopsis promoters include at least one of SEQ ID NOs: 1-6 and/or 233-238. Exemplary Brassica napus promoters include at least one of SEQ ID NOs: 239-279. Exemplary Glycine max promoters include at least one of SEQ ID NOs: 280-313. Exemplary Oryza sativa promoters include at least one of SEQ ID NOs: 314-339. Exemplary Sorghum bicolor promoters include at least one of SEQ ID NOs: 340-377. Exemplary Thlaspi arvense promoters include at least one of SEQ ID NOs: 378-388. Exemplary Zea mays promoters include 389-411. In some examples, the promoter or promoters are operably linked to a target gene or a transgene of interest (such as wild-type MYB41 gene, homologs of MYB41, orthologs of MYB41 and/or paralogs of MYB41, and/or fragments and variations thereof) for increased suberin levels by altering gene expression patterns in a cell-type specific manner.

In some examples, the plant, plant part, or plant cell thereof produces increased levels of suberin as compared to an untransformed control plant. In some examples, a transformed plant tissue is produced from the transformed plant cell. In some examples, a transformed plantlet is produced from the transformed plant tissue and wherein the transformed plantlet produces increased levels of suberin as compared to an untransformed control plantlet. In some examples, a progeny of the transformed plantlet is produced and wherein the progeny produces increased levels of suberin as compared to an untransformed control plantlet. In some examples, the transformed plantlet or the progeny of the transformed plantlet is grown into a mature transformed plant, and wherein the mature transformed plant produces increased levels of suberin as compared to a mature untransformed control plant. In some examples, the mature transformed plant or clone of the mature transformed plant is used in a breeding method taught herein.

In some examples, the plant, plant part, or plant cell of the present disclosure has at least about 5 more, about 10% more, about 15% more, about 20% more, about 25% more, about 30% more, about 35% more, about 40% more, about 45% more, about 50% more, about 60% more, about 70% more, about 80% more, about 90% more, about 100% more, about 200% more, suberin monomers as compared to an untransformed control plant. In some examples, the plant, plant part, or plant cell is a genetically-modified, genetically-engineered, or gene-edited one.

In some examples, the disclosure provides a method of producing a plant having increased suberin production The method can include crossing the genetically-modified or gene-edited plant of the disclosure with itself or another plant; and selecting a progeny plant having increased suberin production. In some examples, the selecting comprises using molecular markers that are designed to detect at least a portion of a sequence selected from SEQ ID NOs: 92-201.

In other examples, the method further includes using the selected progeny in a breeding method taught herein.

In some examples, the disclosure provides a method for reducing carbon dioxide emissions from a soil comprising: growing the gene-edited plant of the disclosure, or a progeny thereof in the soil.

In some examples, the disclosure provides a method of bioremediating soil. The method can include growing the gene-edited plant of the disclosure, or progeny thereof, in soil affected by carbon dioxide emissions, wherein said growing removes excess carbon dioxide from the soil.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1: Selection of Candidate Promoters for Engineering Increases in Suberin Production

We undertook an extensive investigation searching for promoters that might drive more suberin production. More specifically, to engineer more suberin in specific plant layers, we searched for promoters with root cell-type specific expression that might drive suberin-related genes resulting in enhanced suberin production in the roots of transformed plants.

As part of this effort, we assessed the expression patterns of two genes believed to be required for the biosynthesis of suberin, HORST (Vishwanath et al., 2015; Höfer et al., 2008; Wei et al., 2020) and FACT (Kosma, 2012; Molina et al., 2009) by generating promoter fusions with dual GUS-GFP reporters (FIGS. 1A and 2A). For each reporter (“proFACT” or “proHORST”, respectively) three independently generated Arabidopsis lines were characterized and shown to have normal root growth with high GUS expression in the periderm and endodermis of 14-day old roots (FIGS. 1B-D and FIGS. 2B-D). While some weak expression was observed in cortex and epidermis cells, no expression was detected in rosette leaves suggesting that these promoters show minimal expression in cells that are not associated with normal suberin production.

Following similar experimental procedures, five additional promoters were also shown to have GUS expression in the periderm and endodermis. These additional promoters are proASFT, proRALPH, proMYB84, proHORST and proGPAT5. ProASFT, proRALPH, proHORST and proGPAT5 had no GUS expression in rosette leaves, while proMYB84 had some rosette leaf expression.

### Example 2: Use of ProFACT to Drive Ectopic Expression of MYB41 Transcription Factor

The promoter of FACT was used to drive ectopic expression of the MYB41 transcription factor, which was previously shown to induce the production of suberin in tobacco leaves (Kosma et al., 2014), in a root-layer specific manner. Using the proFACT::MYB41 construct (FIG. 2E) (SEQ ID NO: 7) and agrobacterium-mediated transformation using a floral dip method (S. J. Clough and A. F. Bent, 2008, Foral dip: a simplified method for Agrobacterium-mediated transformation of Arabidopsis thaliana, The Plant Journal 16 (6), 33 pages), four independently generated Arabidopsis lines were characterized and shown to express MYB41 (FIG. 2F) and have normal root growth (FIG. 2G).

The effects of this construct on suberin deposition were assessed in these lines by Nile Red staining and confocal imaging. These analyses demonstrate that for four independent, homozygous T3 lines (LS110-LS621, LS112-LS214, LS108-MR219 and LS107-LS442) and two sibling lines (LS107-LS442 and LS107-LS444) an additional Nile Red stained periderm layer is being formed as compared to a wild-type control (Col-0) (FIG. 2H). Thus, we have identified a highly reproducible means of increasing the levels of suberin by generating additional periderm cells as compared to wild-type plants.

### Example 3: Use of ProHORST to Drive Ectopic Expression of MYB41 Transcription Factor

The promoter of HORST was used to drive ectopic expression of the MYB41 transcription factor, which was previously shown to induce the production of suberin in tobacco leaves (Kosma et al., 2014), in a root-layer specific manner. Using the proHORST::MYB41 construct (FIG. 1E) (SEQ ID NO: 8) and agrobacterium-mediated transformation (Clough and Brent, 2008), two independently generated Arabidopsis lines were characterized and shown to have normal root growth (FIG. 1F).

The effects of this construct on suberin deposition were assessed in these lines by Nile Red staining and confocal imaging. In this case we found that in two independent, homozygous T3 lines (LS140-LS766 and LS141-LS788) and two sibling lines (LS140-LS766 and LS140-LS767) more Nile Red signal is observed in the periderm as compared with a wild-type control (Col-0) (FIG. 1G). Thus, we have identified a highly reproducible means of increasing the levels of suberin by depositing more suberin in existing periderm cells as compared to wild-type plants.

### Example 4: Quantification of Suberin Biomarkers

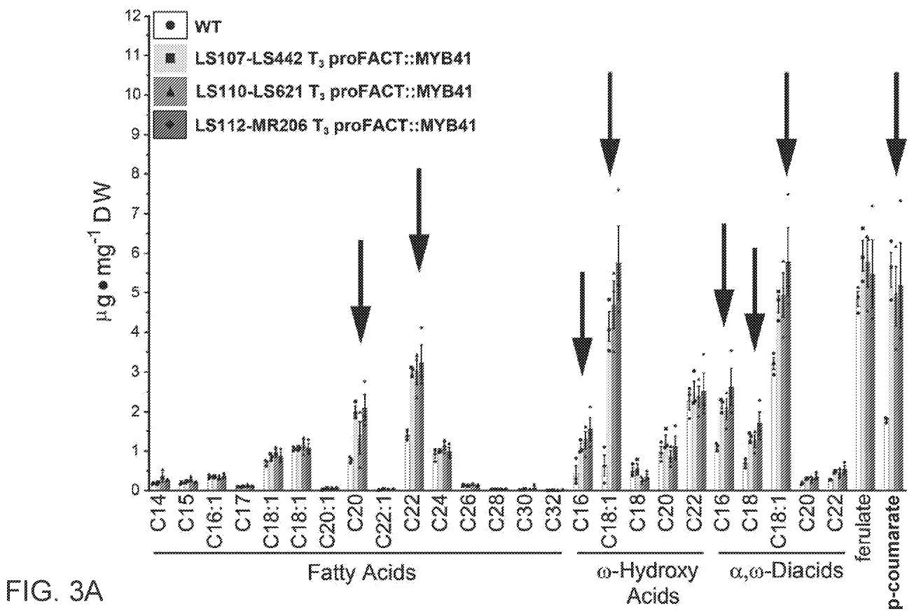

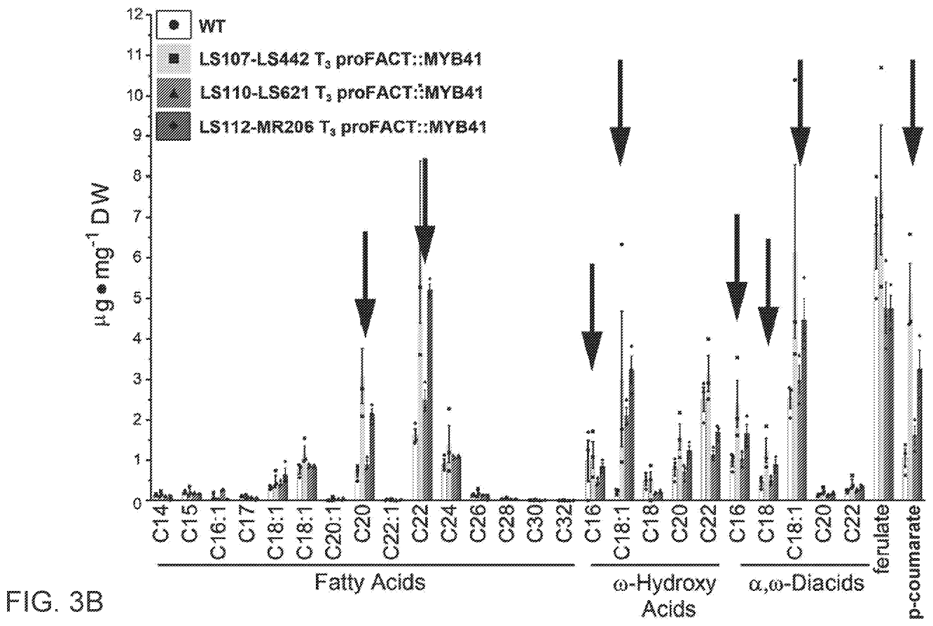

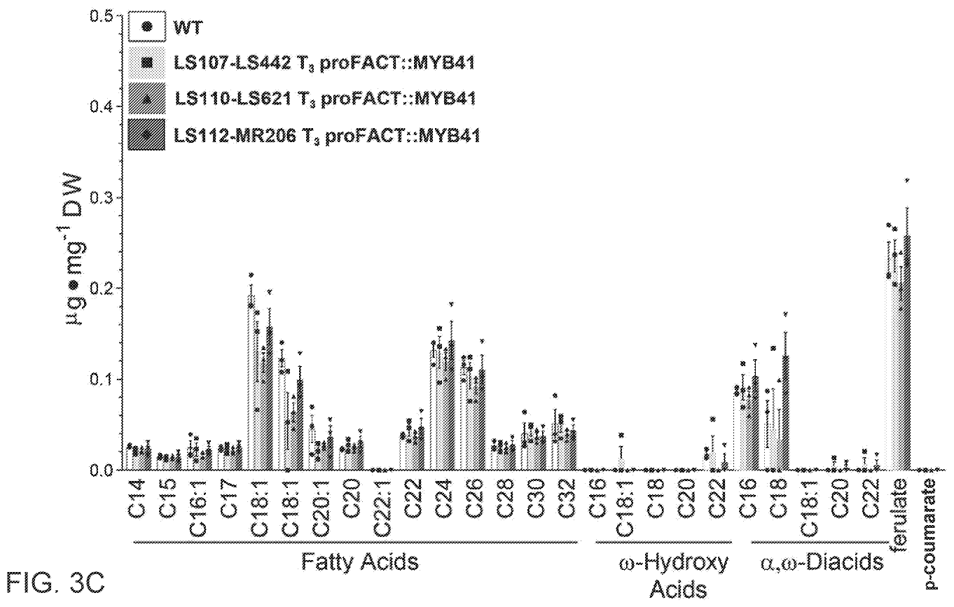

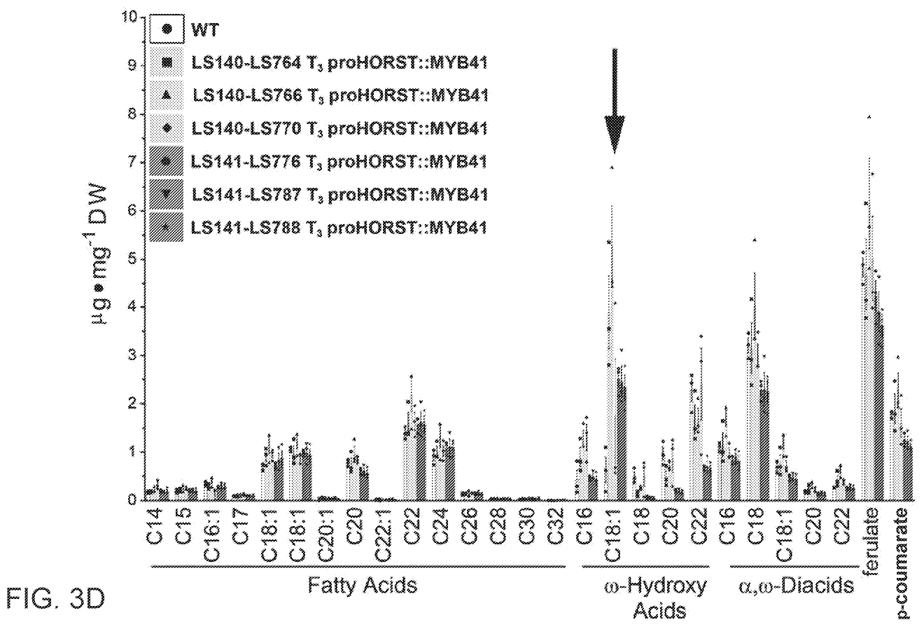

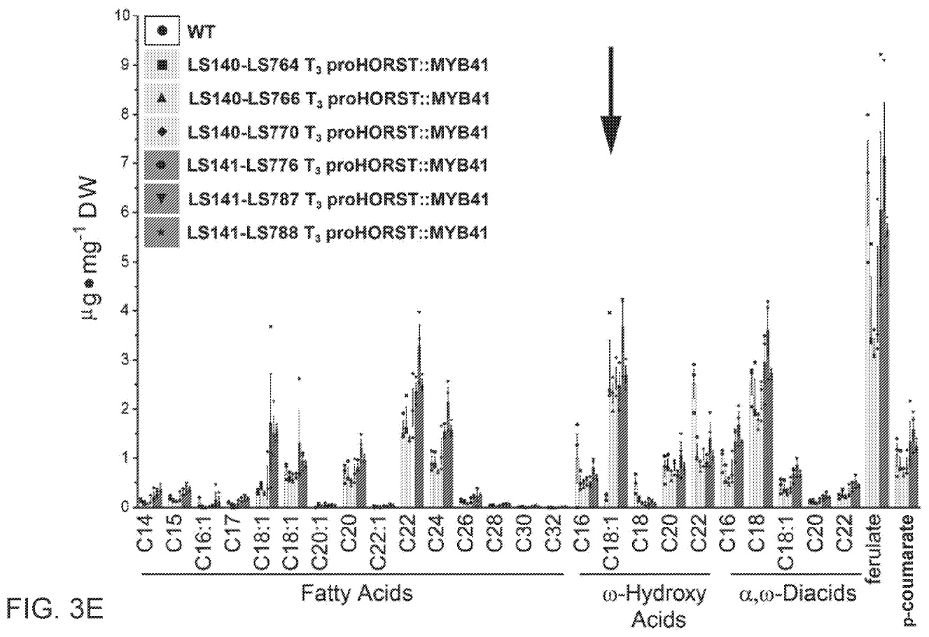

To quantify the levels of suberin biomarkers in the proFACT::MYB41 and proHORST::MYB41 lines that had more periderm layers and/or increased Nile Red staining, reactive pyrolysis-gas chromatography-mass spectrometry (RxPyGC-MS) experiments were conducted using dried root tissue from either 14 or 28 day old seedlings. For each line, three technical replicates were included, with the average values and the standard error of the mean plotted for the abundances of select fatty acids, ω-hydroxy acids, α,ω-diacids, and phenolics associated with suberin composition (FIGS. 3A-3E).

For proFACT::MYB41, three independent T3 homozygous lines were assayed (FIG. 3A and FIG. 3B) and at both the 14 and 28 day time points, these lines consistently showed increased levels of many suberin biomarkers. For these same lines, RxPyGC-MS was also conducted using dried shoot tissue from 14 day old seedlings and no increases in suberin biomarkers were observed (FIG. 3C). These findings are consistent with upregulation of many key steps of suberin biosynthesis specifically in plant roots. Furthermore, the data from the 28 day old root samples indicate that at maturity the engineered lines contain more suberin compared to non-engineered wild-type control lines.

For proHORST::MYB41, three sibling lines from two independent T3 homozygous parents were assayed (FIG. 3D and FIG. 3E) and at both the 14 and 28 day time points, these lines consistently showed increased levels of one suberin biomarker, C18:1 ω-hydroxy acid. Given that these lines show more intense Nile Red staining in periderm tissue, these findings suggest that production of C18:1 ω-hydroxy acids may be partially rate limiting in the suberin biosynthetic pathways. Overall, these analyses of the pFACT::MYB41 and pHORST::MYB41 lines demonstrate that depending on where MYB41 is expressed, different patterns of suberin staining can be generated in vivo and that these changes are associated with different suberin biomarker signatures specifically in root tissue.

### Example 5: Use of Additional Promoters to Drive Ectopic Expression of MYB41 Transcription Factor

The promoters of ASFT, GPAT5, RALPH and MYB84 can each be used to drive ectopic expression of the MYB41 transcription factor, which was previously shown to induce the production of suberin in tobacco leaves (Kosma et al., 2014), in a root-layer specific manner. Using any one or more of proASFT::MYB41, proGPAT5::MYB41, proRALPH::MYB41 and proMYB84:MYB41 constructs (SEQ ID NO: 9, SEQ ID NO: 10, SEQ ID NO: 11 and SEQ ID NO: 12, respectively) and agrobacterium-mediated transformation, independently generated Arabidopsis lines can be characterized and shown to have normal root growth.

The effects of these constructs on suberin deposition can be assessed in these lines by Nile Red staining and confocal imaging. Using this process, one can find independent, homozygous T3 lines and sibling lines in which more Nile Red signal is observed in the periderm as compared with a wild-type control (Col-0). Thus, in this way we provided a highly reproducible means of increasing the levels of suberin by depositing more suberin in existing periderm cells as compared to wild-type plants.

### Example 6. Use of Promoters to Drive Ectopic Expression of MYB41 Transcription Factor in Canola (Brassica napus)

Any one or more of FACT, HORST, ASFT, GPAT5, RALPH and MYB84 promoters derived or isolated from Arabidopsis, canola, or other plant species can be used to drive ectopic expression of a canola MYB41 transcription factor or a MYB41 ortholog in canola (from SEQ ID NOs: 37-48) in a root-layer specific manner. Vectors comprising one or more canola-derived FACT, HORST, ASFT, GPAT5, RALPH and MYB84 promoters operably linked to one of more MYB41 orthologs in canola will be generated and then transformed by agrobacterium-mediated transformation (Kosma et al., 2014). The genetically-engineered canola lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 7: Use of Promoters to Drive Ectopic Expression of MYB41 Transcription Factor in Soybean (Glycine max)

Any one or more of FACT, HORST, ASFT, GPAT5, RALPH and MYB84 promoters derived or isolated from Arabidopsis, soybean, or other plant species can be used to drive ectopic expression of a soybean MYB41 transcription factor or a MYB41 ortholog in soybean (from SEQ ID NOs: 49-56) in a root-layer specific manner. Vectors comprising one or more soybean-derived FACT, HORST, ASFT, GPAT5, RALPH and MYB84 promoters operably linked to one of more MYB41 orthologs in soybean will be generated and then transformed by agrobacterium-mediated transformation (Kosma et al., 2014). The genetically-engineered soybean lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 8: Use of Promoters to Drive Ectopic Expression of MYB41 Transcription Factor in Cotton (Gossypium hirsutum)

Any one or more of FACT, HORST, ASFT, GPAT5, RALPH and MYB84 promoters derived or isolated from Arabidopsis, cotton, or other plant species can be used to drive ectopic expression of a cotton MYB41 transcription factor or a MYB41 ortholog in cotton (from SEQ ID NOs: 59-68) in a root-layer specific manner. Vectors comprising one or more cotton-derived FACT, HORST, ASFT, GPAT5, RALPH and MYB84 promoters operably linked to one of more MYB41 orthologs in cotton will be generated and then transformed by agrobacterium-mediated transformation (Kosma et al., 2014). The genetically-engineered cotton lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 9: Use of Promoters to Drive Ectopic Expression of MYB41 Transcription Factor in Rice (Oryza sativa)

Any one or more of FACT, HORST, ASFT, GPAT5, RALPH and MYB84 promoters derived or isolated from Arabidopsis, rice, or other plant species can be used to drive ectopic expression of a rice MYB41 transcription factor or a MYB41 ortholog in rice (from SEQ ID NO: 69) in a root-layer specific manner. Vectors comprising one or more rice-derived FACT, HORST, ASFT, GPAT5, RALPH and MYB84 promoters operably linked to one of more MYB41 orthologs in rice will be generated and then transformed by agrobacterium-mediated transformation (Kosma et al., 2014). The genetically-engineered rice lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

Example 10: Use of Promoters to Drive Ectopic Expression of MYB41 Transcription Factor in Lotus (Lotus japonicus)

Any one or more of FACT, HORST, ASFT, GPAT5, RALPH and MYB84 promoters derived or isolated from Arabidopsis, lotus, or other plant species can be used to drive ectopic expression of a lotus MYB41 transcription factor or a MYB41 ortholog in lotus (from SEQ ID NOs: 70-72) in a root-layer specific manner. Vectors comprising one or more lotus-derived FACT, HORST, ASFT, GPAT5, RALPH and MYB84 promoters operably linked to one of more MYB41 orthologs in lotus will be generated and then transformed by agrobacterium-mediated transformation (Kosma et al., 2014). The genetically-engineered lotus lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 11: Use of Promoters to Drive Ectopic Expression of MYB41 Transcription Factor in Radish (Raphanus sativus)

Any one or more of FACT, HORST, ASFT, GPAT5, RALPH and MYB84 promoters derived or isolated from Arabidopsis, radish, or other plant species can be used to drive ectopic expression of a radish MYB41 transcription factor or a MYB41 ortholog in radish (from SEQ ID NOs: 73-81) in a root-layer specific manner. Vectors comprising one or more radish-derived FACT, HORST, ASFT, GPAT5, RALPH and MYB84 promoters operably linked to one of more MYB41 orthologs in radish will be generated and then transformed by agrobacterium-mediated transformation (Kosma et al., 2014). The genetically-engineered radish lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 12: Use of Promoters to Drive Ectopic Expression of MYB41 Transcription Factor in Setaria (Setaria italica)

Any one or more of FACT, HORST, ASFT, GPAT5, RALPH and MYB84 promoters derived or isolated from Arabidopsis, setaria, or other plant species can be used to drive ectopic expression of a setaria MYB41 transcription factor or a MYB41 ortholog in setaria (from SEQ ID NO: 82) in a root-layer specific manner. Vectors comprising one or more setaria-derived FACT, HORST, ASFT, GPAT5, RALPH and MYB84 promoters operably linked to one of more MYB41 orthologs in setaria will be generated and then transformed by agrobacterium-mediated transformation (Kosma et al., 2014). The genetically-engineered setaria lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 13: Use of Promoters to Drive Ectopic Expression of MYB41 Transcription Factor in Sorghum (Sorghum bicolor)

Any one or more of FACT, HORST, ASFT, GPAT5, RALPH and MYB84 promoters derived or isolated from Arabidopsis, sorghum, or other plant species can be used to drive ectopic expression of a sorghum MYB41 transcription factor or a MYB41 ortholog in sorghum (from SEQ ID NO: 83) in a root-layer specific manner. Vectors comprising one or more sorghum-derived FACT, HORST, ASFT, GPAT5, RALPH and MYB84 promoters operably linked to one of more MYB41 orthologs in sorghum will be generated and then transformed by agrobacterium-mediated transformation (Kosma et al., 2014). The genetically-engineered sorghum lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 14: Use of Promoters to Drive Ectopic Expression of MYB41 Transcription Factor in Pennycress (Thlaspi arvense)

Any one or more of FACT, HORST, ASFT, GPAT5, RALPH and MYB84 promoters derived or isolated from Arabidopsis, pennycress, or other plant species can be used to drive ectopic expression of a pennycress MYB41 transcription factor or a MYB41 ortholog in pennycress (from SEQ ID NOs: 84-86) in a root-layer specific manner. Vectors comprising one or more pennycress-derived FACT, HORST, ASFT, GPAT5, RALPH and MYB84 promoters operably linked to one of more MYB41 orthologs in pennycress will be generated and then transformed by agrobacterium-mediated transformation (Kosma et al., 2014). The genetically-engineered pennycress lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 15: Use of Promoters to Drive Ectopic Expression of MYB41 Transcription Factor in Southern Cattail (Typha domingensis)

Any one or more of FACT, HORST, ASFT, GPAT5, RALPH and MYB84 promoters derived or isolated from Arabidopsis, southern cattail, or other plant species can be used to drive ectopic expression of a southern cattail MYB41 transcription factor or a MYB41 ortholog in southern cattail (from SEQ ID NO: 87) in a root-layer specific manner. Vectors comprising one or more southern cattail-derived FACT, HORST, ASFT, GPAT5, RALPH and MYB84 promoters operably linked to one of more MYB41 orthologs in southern cattail will be generated and then transformed by agrobacterium-mediated transformation (Kosma et al., 2014). The genetically-engineered southern cattail lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 16: Use of Promoters to Drive Ectopic Expression of MYB41 Transcription Factor in Wheat (Triticum aestivum)

Any one or more of FACT, HORST, ASFT, GPAT5, RALPH and MYB84 promoters derived or isolated from Arabidopsis, wheat, or other plant species can be used to drive ectopic expression of a wheat MYB41 transcription factor or a MYB41 ortholog in wheat (from SEQ ID NOs: 88-89) in a root-layer specific manner. Vectors comprising one or more wheat-derived FACT, HORST, ASFT, GPAT5, RALPH and MYB84 promoters operably linked to one of more MYB41 orthologs in wheat will be generated and then transformed by agrobacterium-mediated transformation (Kosma et al., 2014). The genetically-engineered wheat lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, inventors can provide a highly reproducible means of increasing the levels of suberin by depositing more suberin in existing periderm cells as compared to wild-type plants.

### Example 17: Use of Promoters to Drive Ectopic Expression of MYB41 Transcription Factor in Maize (Zea mays)

Any one or more of FACT, HORST, ASFT, GPAT5, RALPH and MYB84 promoters derived or isolated from Arabidopsis, maize, or other plant species can be used to drive ectopic expression of a maize MYB41 transcription factor or a MYB41 ortholog in maize (from SEQ ID NOs: 90-91) in a root-layer specific manner. Vectors comprising one or more maize-derived FACT, HORST, ASFT, GPAT5, RALPH and MYB84 promoters operably linked to one of more MYB41 orthologs in maize will be generated and then transformed by agrobacterium-mediated transformation (Kosma et al., 2014). The genetically-engineered maize lines can be characterized for changes of root biomass or structure as well as suberin content.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. In this way, provided is a highly reproducible means of increasing the levels of suberin by depositing more suberin in existing periderm cells as compared to wild-type plants.

### Example 18: Quantification of Total Shoot Weight

As described above, heterologous promoters can drive ectopic expression of MYB41 transcription factor in pennycress (Thlaspi arvense). The AtMYB41 transcription factor was overexpressed in pennycress to increase suberin content in the roots. The vector included the Arabidopsis genomic MYB41 driven by the Arabidopsis HORST promoter (proHORST::MYB41). The plasmid had a BASTA resistance marker. T1 seeds were collected from the transformation, and transformed pennycress lines were identified among the T1 seeds.

T1 seeds were screened by plating them on ½ MS plates containing BASTA, and subsequently transplanting potential positive plants onto soil. Plants were then genotyped to verify the presence of the transgene. T2 plants were genotyped, phenotyped, and grown in a greenhouse to maturity. T2 seeds were collected, and progeny tests were performed on the T3 generation to identify plants homozygous plants for the transgene. Sibling negative controls which did not contain the transgene were also identified.

The effect of proHORST::MYB41 expression on the total shoot biomass of pennycress plants was analyzed. T2 seeds were liquid sterilized (with 70% ethanol+0.5% Tween20 solution for 20 minutes), then plated on Murashige-Skoog media (1× strength, 1% sucrose, 0.8% phytagel, 0.01% plant preservative mixture) and stratified for one week. The stratified seeds were transferred to a Conviron (22° C., 50% humidity, 16 h day) for seven days. The seedlings were transplanted to wetted Jiffy Peat Pellets and grown in a greenhouse for 14 days. At 14 days, trays with Jiffy were vernalized in a Conviron (4° C., 100% humidity, no light) for two weeks. After vernalization, the Jiffy with seedlings were transplanted into a 5-gal black pot filled with turface, grown in a greenhouse, and watered with Peters Excel® 15-5-15 Fertilizer.

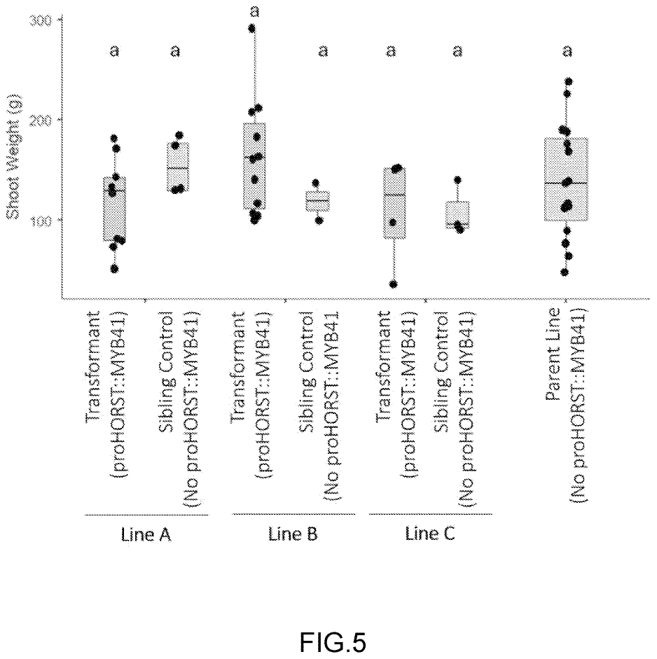

Primers specific to the proHORST: MYB41 construct were use to genotype the T2 segregating population via PCR assays. Plants were selected from this population with the transgene (both homozygous and heterozygous) and plants lacking the transgene, which served as sibling controls. To study the total shoot biomass, mature proHORST::MYB41 transgenic lines, their sibling controls, and the parent line were harvested and dried (FIG. 5). The parent line was an agronomically improved plant that was transformed to generate the proHORST::MYB41 lines. No significant differences in total shoot biomass were observed between transgenic lines, sibling controls, and the parent line (FIG. 5).

### Example 19: Quantification of Suberin Content Via pyGCMS

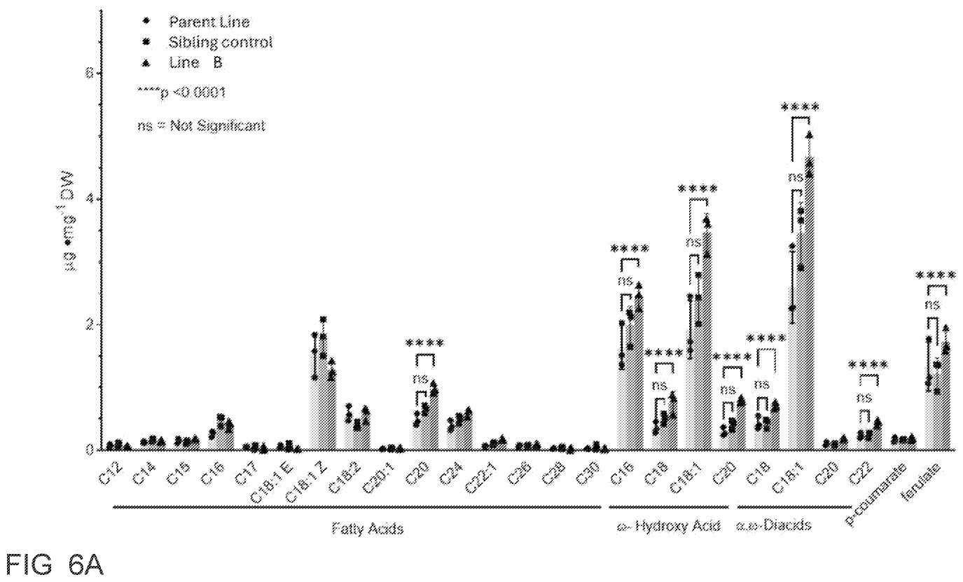

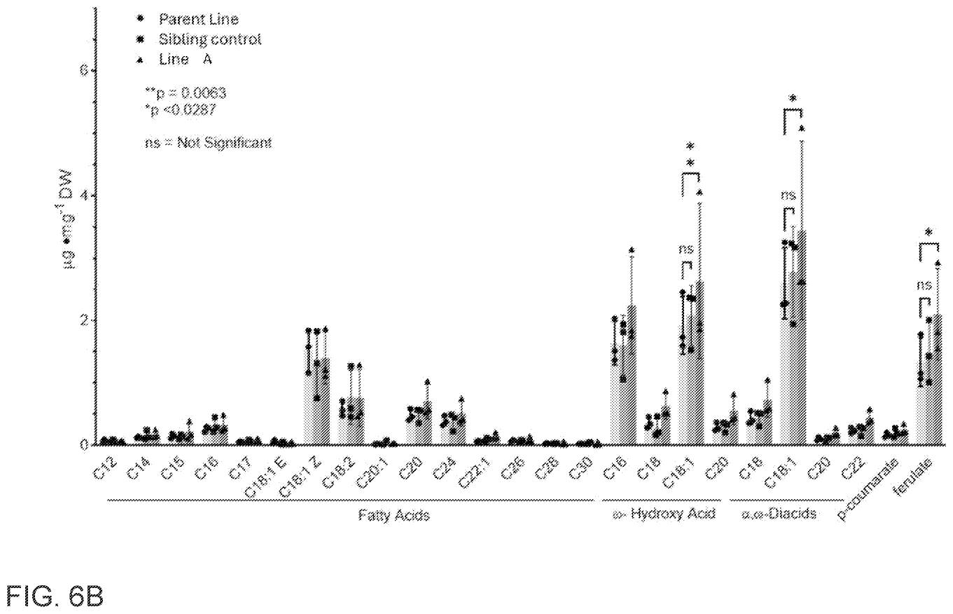

As described in Example 18, proHORST::MYB41 pennycress lines were established. Methyl esters of C12-C30 carbon chain fatty acids, C16-C20 @-hydroxy acids, C18-C22 α,ω-dicarboxyacids as well as the two phenolic compounds, p-coumarate and ferulate, were detected and quantified by reactive pyrolysis gas chromatography mass spectrometry (RxPyGCMS). For these experiments, mature lateral roots from the following lines were analyzed: proHORST::MYB41 transgenic homozygous plant of Line B, Line A, their respective non-transgenic sibling control lines, and the parent line (FIGS. 6A, 6B). Measurements were made from direct tissue pyrolysis, so the quantified amounts are expected to be a sum of fatty acids from lipids and suberin derived monomers. Prior work within HPI, measuring temporal changes in the accumulation of suberin monomers in pennycress indicated that lipid derived fatty acids decrease as the roots mature, while the very long chain fatty acids (VLCFAs) and functional groups containing fatty acids, ω-hydroxy and α,ω-dicarboxyacids, that are thought to be suberin biomarkers, increase. Consistent with this idea, almost all of the measured ω-hydroxy and α,ω-dicarboxyacids, with the exception of C20 α,ω-diacid, showed a significant increase (p<0.029) in both the proHORST::MYB41 overexpression lines, when compared to the parent line and the sibling control. This result was verified in two additional independent proHORST::MYB41 homozygous transgenic lines indicating that the engineered lines contain significantly higher suberin content compared to the agronomically improved pennycress parent line use to create these transformants.

Tissue pyrolysis was performed as follows. 0.2 mg dried homogenized root tissue collected from lateral roots of mature pennycress genotypes were analyzed by reactive pyrolysis gas chromatography mass spectrometry (RxPyGCMS) on a Shimadzu QP2020-NX GCMS equipped with a Frontier multishot EGA PY3030D pyrolizer. 5 μL of tetramethyl ammonium hydroxide (TMAH) and 0.5 μg of (z)-10-pentadecenoic acid (C15:1) methyl ester internal standard was added and the root material was incubated for 30 mins prior to loading leading on the pyrolizer. Pyrolysis was performed at 350° C. with an inlet temperature set to 300° C. and the sample was introduced with a split flow of 5:1. Volatilized compounds were separated on an SH-Rxi-5 ms column (Shimadzu, 30 m, 0.25 mm id) at a column flow of 1 mL min⁻¹. A temperature gradient of 60° C. to 320° C. was used for separation with a ramp of 6° C. min⁻¹, then held at 320° C. for 5 min. Mass spectra was collected from 50-700 m/z with ion source temperature of 200° C. Suberin monomer concentrations were calculated using extracted ion chromatograms, with external calibration curves generated from standard mixes containing methyl esters of fatty acids, α,ω-diacids, ω-hydroxy acids, p-coumaric acid, and ferulic acid (Sigma, Cayman, and Matreya), relative to the internal standard, C15:1. For 1,18-octadec-9-enedioic acid and 18-hydroxy octadic-9-enoic acid, where commercial standards are not available, identifications are made by comparing collected MS spectra to EI spectra reported in the lipid web and quantified by area ratio to a known concentration of the internal standard.

### Example 20: MYB41 Expression Analysis

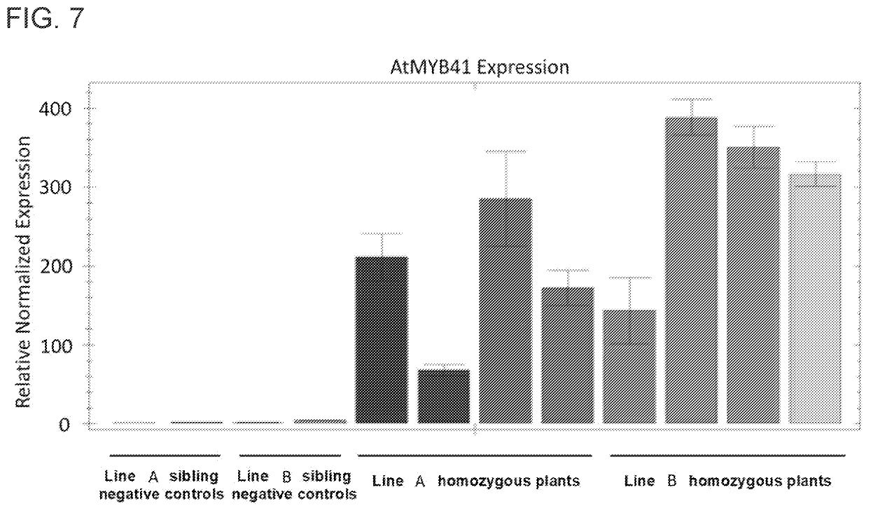

As described in Example 18, proHORST::MYB41 pennycress lines were established. After identifying negative sibling controls and homozygous proHORST::MYB41 lines by genotyping, several lines were plated on ½ MS plates after sterilizing seeds. Plants were grown for ~4 weeks (approximately when periderm development begins). The top half (where periderm/suberin are usually present) of the roots was cut and RNA was extracted from the top half of the roots of from several lines of plants using the Zymo Direct-zol RNA Miniprep Kit. cDNA was then synthesized using the SuperScript III First-Strand Synthesis SuperMix (Thermo/Invitrogen) kit. qPCR was then performed using the Luna Universal qPCR Master Mix (NEB) to determine expression levels of AtMYB41 in root tissue. As shown in FIG. 7, expression of AtMYB41 in sibling control plants was not detected. By contrast, homozygous lines showed high expression, indicating that the Arabidopsis HORST promoter can drive the expression of AtMYB41 in pennycress roots.

The results of Examples 18, 19 and 20 demonstrate that the AtMYB41 gene expressed using the HORST promoter significantly increases AtMYB41 expression in the roots of pennycress plants compared to non-transgenic sibling controls (FIG. 7). This increase in MYB41 does not impact total shoot biomass, as there is no notable difference in biomass among transformed plants, their sibling controls, and parent lines (FIG. 5). Analysis using pyGCMS demonstrates considerably higher suberin monomers in the roots of the transgenic lines with the proHORST::MYB41 construct compared to non-transgenic sibling controls or parent lines (FIGS. 6A, 6B). These findings demonstrate that AtMYB41 expressed under the AtHORST promoter significantly increases suberin levels in roots without affecting the total shoot biomass of the pennycress plant.

### Example 21: Generation of Ectopic Expression Vectors and Use to Drive Ectopic Expression of MYB41 Transcription Factor in Pennycress (Thlaspi arvense)

Using the methods described above, six additional vectors were generated in which six different Arabidopsis promoters (proFACT, proHORST, proRALPH, proASFT, proGPAT5, proMYB84) control the expression of Arabidopsis genomic MYB41. Cassettes were cloned into a vector containing the DsRed marker for easier selection. Vectors were transformed into pennycress plants (Thlaspi arvense), T1 seeds were collected from the transformation, and T2 seeds were generated from the T1 plants.

The effects of these constructs on suberin deposition can be assessed in these lines by Nile Red staining and confocal imaging. Using this process, one can find independent, homozygous lines and sibling lines in which more Nile Red signal is observed in the periderm as compared with a wild-type control. This provides a highly reproducible means of increasing the levels of suberin by depositing more suberin in existing periderm cells as compared to wild-type plants.

The effects of these constructs on suberin deposition can also be assessed using qPCR. Plants can be grown for a suitable length of time, such as 1, 2, 3, 4, 5, or more weeks, and an aspect of the root tissue can be isolated, such as an aspect including the epidermis, epidermal hair cells, the cortext, the endodermis, the pericycle, the periderm, the xylem, the phloem, the stele, and/or the columella. RNA can be extracted from the isolated aspect, and cDNA can be synthesized from the RNA. qPCR can then be performed to assess expression levels of AtMYB41 in the isolated aspect of the roots.

The effects of these constructs on suberin deposition can also be assessed using pyGCMS. Plants can be grown for a suitable length of time, such as 1, 2, 3, 4, 5, or more weeks, and an aspect of the root tissue can be isolated, such as an aspect including the epidermis, epidermal hair cells, the cortext, the endodermis, the pericycle, the periderm, the xylem, the phloem, the stele, and/or the columella. pyGCMS can be performed to assess the presence of suberin monomers in the isolated aspect.

### Further Numbered Examples of the Disclosure

Other subject matter contemplated by the present disclosure is set out in the following numbered examples:

### MYB41 Orthologs

1. An isolated nucleic acid molecule comprising a nucleic acid sequence encoding a MYB41 amino acid sequence with at least 80% sequence homology to any one of SEQ ID NOs: 37-91 and/or a nucleic acid sequence set forth with at least 80% sequence homology to any one of SEQ ID NOs: 92-201, operably linked to a nucleic acid sequence encoding a heterologous promoter, wherein expression of the isolated nucleic acid molecule in a plant results in increased levels of suberin as compared to wild-type check plants lacking the isolated nucleic acid molecule.
2. The isolated nucleic acid molecule of example 1, wherein the increased levels of suberin occur by generating additional periderm cells and/or depositing more suberin in existing periderm cells.
3. The isolated nucleic acid molecule of example 1 or example 2, wherein the amino acid sequence homology is selected from the group consisting of at least 85% homology, at least 90% homology, at least 95% homology, at least 96% homology, at least 97% homology, at least 98% homology and at least 99% homology to any one of SEQ ID NOs: 37-91.
4. The isolated nucleic acid molecule of example 1 or example 2, wherein the amino acid sequence is any one of SEQ ID NOs: 37-91.
5. The isolated nucleic acid molecule of any one of examples 1-4, wherein the nucleic acid sequence is any one of SEQ ID NOs: 92-201.
6. A transformation vector comprising one or more of the nucleic acid molecules of any one of examples 1-5 and 22-28.
7. A method of transforming a plant cell, the method comprising introducing the transformation vector of example 6 into a plant cell, thereby generating a transformed plant cell, whereby the transformed plant cell produces increased levels of suberin as compared to an untransformed wild-type check plant cell.
8. The method of example 7, further comprising producing transformed plant tissue from the transformed plant cell.
9. The method of example 8, further comprising producing a transformed plantlet from the transformed plant tissue, wherein the transformed plantlet produces increased levels of suberin as compared to untransformed wild-type check plantlets lacking the isolated nucleic acid molecule.
10. The method of example 9, further comprising producing a progeny of the transformed plantlet, wherein the progeny produces increased levels of suberin as compared to untransformed wild-type check plantlets lacking the isolated nucleic acid molecule.
11. The method of example 9 or example 10, further comprising growing the transformed plantlet or progeny of the transformed plantlet into a mature transformed plant, wherein the mature transformed plant produces increased levels of suberin as compared to mature untransformed wild-type checks lacking the isolated nucleic acid molecule.
12. The method of any one of examples 9-11, wherein the increased levels of suberin occur by generating additional periderm cells and/or depositing more suberin in existing periderm cells.
13. The method of any one of examples 9-12, wherein there is minimal or no expression of the nucleic acid molecule in cells that are not associated with normal suberin production.
14. The method of any one of examples 9-13, wherein there is minimal or no expression of the nucleic acid molecule in rosette leaves.
15. The method of any one of examples 11-14, further comprising using the mature transformed plant or clone of the mature transformed plant in a breeding method.
16. The method of example 15, wherein the breeding method comprises selfing or crossing the mature transformed plant or clone of the mature transformed plant.
17. A plant breeding method comprising crossing a first plant comprising a nucleic acid molecule of any one of examples 1-5 and 22-28 with a second plant of the same species and selecting resultant progeny of the cross based on increased levels of suberin as compared to wild-type check plants.
18. The plant breeding method of example 17, further comprising producing clones of the resultant progeny of the cross wherein the clones are selected based on increased levels of suberin as compared to wild-type check plants.
19. The plant breeding method of example 17 or example 18, wherein the progeny of the cross that display increased levels of suberin as compared to wild-type check plants are selected using molecular markers that are designed based on the nucleic acid molecule of any one of examples 1-5 and 22-28.
20. The method of example 17, further comprising using the selected progeny in a breeding method.
21. The method of any one of examples 11-14, further comprising growing the mature transformed plant or clone of the mature transformed plant in a greenhouse or outdoors.
22. The isolated nucleic acid molecule of any one of examples 1-5, wherein the heterologous promoter comprises an isolated nucleic acid sequence set forth in any one of SEQ ID NO: 1, SEQ ID NO: 2, SEQ ID NO: 3, SEQ ID NO: 4, SEQ ID NO: 5, SEQ ID NO: 6 and SEQ ID NOs: 233-411, or a sequence at least 70%, at least 80%, at least 90%, at least 95%, at least 98%, or at least 99% identical thereto.
23. The isolated nucleic acid molecule of any one of examples 1-5, wherein the heterologous promoter is a promoter of FACT gene, such as an isolated nucleic acid sequence set forth in any one of SEQ ID NOs: 1, 234, 239-246, 280-285, 314-316, 340-349, 379, 380, or 389-391, or a sequence at least 70%, at least 80%, at least 90%, at least 95%, at least 98%, or at least 99% identical thereto.
24. The isolated nucleic acid molecule of any one of examples 1-5, wherein the heterologous promoter is a promoter of HORST gene, such as an isolated nucleic acid sequence set forth in any one of SEQ ID NOs: 2, 236, 254-258, 288, 289, 319-321, 352-354, 383, 394, or 395, or a sequence at least 70%, at least 80%, at least 90%, at least 95%, at least 98%, or at least 99% identical thereto.
25. The isolated nucleic acid molecule of any one of examples 1-5, wherein the heterologous promoter is a promoter of ASFT gene, such as an isolated nucleic acid sequence set forth in any one of SEQ ID NOs: 3, 233, 239-244, 280-285, 314-316, 340-349, 378, 379, or 389-391, or a sequence at least 70%, at least 80%, at least 90%, at least 95%, at least 98%, or at least 99% identical thereto.
26. The isolated nucleic acid molecule of any one of examples 1-5, wherein the heterologous promoter is a promoter of GPAT5 gene, such as an isolated nucleic acid sequence set forth in any one of SEQ ID NOs: 4, 235, 247-253, 286, 287, 317, 318, 350, 351, 381, 382, 392, or 393, or a sequence at least 70%, at least 80%, at least 90%, at least 95%, at least 98%, or at least 99% identical thereto.
27. The isolated nucleic acid molecule of any one of examples 1-5, wherein the heterologous promoter is a promoter of RALPH gene, such as an isolated nucleic acid sequence set forth in any one of SEQ ID NOs: 5, 238, 387, or 388, or a sequence at least 70%, at least 80%, at least 90%, at least 95%, at least 98%, or at least 99% identical thereto.
28. The isolated nucleic acid molecule of any one of examples 1-5, wherein the heterologous promoter is a promoter of MYB84 gene, such as an isolated nucleic acid sequence set forth in any one of SEQ ID NOs: 6, 237, 259-279, 290-313, 322-339, 355-377, 384-386, or 396-411, or a sequence at least 70%, at least 80%, at least 90%, at least 95%, at least 98%, or at least 99% identical thereto.
29. The isolated nucleic acid molecule of example 1, wherein the MYB41 amino acid sequence is present in Canola (Brassica napus) and comprises any one of SEQ ID NOs: 37-48 or a sequence at least 80%, at least 90%, at least 95%, at least 98%, or at least 99% identical thereto.
30. The isolated nucleic acid molecule of example 1, wherein the MYB41 amino acid sequence is present in Soybean (Glycine max) and comprises any one of SEQ ID NOs: 49-56 or a sequence at least 80%, at least 90%, at least 95%, at least 98%, or at least 99% identical thereto.
31. The isolated nucleic acid molecule of example 1, wherein the MYB41 amino acid sequence is present in Cotton (Gossypium hirsutum) and comprises any one of SEQ ID NOs: 57-68 or a sequence at least 80%, at least 90%, at least 95%, at least 98%, or at least 99% identical thereto.
32. The isolated nucleic acid molecule of example 1, wherein the MYB41 amino acid sequence is present in Rice (Oryza sativa) and comprises any one of SEQ ID NO: 69 or a sequence at least 80%, at least 90%, at least 95%, at least 98%, or at least 99% identical thereto.
33. The isolated nucleic acid molecule of example 1, wherein the MYB41 amino acid sequence is present in Lotus (Lotus japonicus) and comprises any one of SEQ ID NOs: 70-72 or a sequence at least 80%, at least 90%, at least 95%, at least 98%, or at least 99% identical thereto.
34. The isolated nucleic acid molecule of example 1, wherein the MYB41 amino acid sequence is present in Radish (Raphanus sativus) and comprises any one of SEQ ID NOs: 73-81 or a sequence at least 80%, at least 90%, at least 95%, at least 98%, or at least 99% identical thereto.
35. The isolated nucleic acid molecule of example 1, wherein the MYB41 amino acid sequence is present in Setaria (Setaria italica) and comprises any one of SEQ ID NO: 82 or a sequence at least 80%, at least 90%, at least 95%, at least 98%, or at least 99% identical thereto.
36. The isolated nucleic acid molecule of example 1, wherein the MYB41 amino acid sequence is present in Sorghum (Sorghum bicolor) and comprises any one of SEQ ID NO: 83 or a sequence at least 80% identical thereto.
37. The isolated nucleic acid molecule of example 1, wherein the MYB41 amino acid sequence is present in Pennycress (Thlaspi arvense) and comprises any one of SEQ ID NOs: 84-86 or a sequence at least 80%, at least 90%, at least 95%, at least 98%, or at least 99% identical thereto.
38. The isolated nucleic acid molecule of example 1, wherein the MYB41 amino acid sequence is present in Southern cattail (Typha domingensis) and comprises any one of SEQ ID NO: 87 or a sequence at least 80%, at least 90%, at least 95%, at least 98%, or at least 99% identical thereto.
39. The isolated nucleic acid molecule of example 1, wherein the MYB41 amino acid sequence is present in Wheat (Triticum aestivum) and comprises any one of SEQ ID NOs: 88-89 or a sequence at least 80%, at least 90%, at least 95%, at least 98%, or at least 99% identical thereto.
40. The isolated nucleic acid molecule of example 1, wherein the MYB41 amino acid sequence is present in Maize (Zea mays) and comprises any one of SEQ ID NOs: 90-91 or a sequence at least 80%, at least 90%, at least 95%, at least 98%, or at least 99% identical thereto.
41. The isolated nucleic acid molecule of example 1, wherein the MYB41 amino acid sequence comprises at least 80%, at least 90%, at least 95%, at least 98%, or at least 99% identical to any one of SEQ ID NOs: 38, 40, 44, 46, 49, 52, 70, 76, 77, 78, and 86.
42. The isolated nucleic acid molecule of example 1, wherein the nucleic acid sequence comprises at least 80% identical to any one of SEQ ID NOs: 93, 95, 99, 101, 104, 107, 125, 131, 132, 133, and 141.
43. The isolated nucleic acid molecule of example 1, wherein the nucleic acid sequence comprises at least 80% identical to any one of SEQ ID NOs: 148, 150, 154, 156, 159, 162, 180, 186, 187, 188, and 196.
44. A plant, a part thereof, or a cell thereof, comprising the isolated nucleic acid molecule of any one of any one of examples 1-5 and 22-43.
45. A plant, a part thereof, or a cell thereof, comprising the transformation vector of example 6.
46. A plant, a part thereof, or a cell thereof, produced by the method of any one of examples 7-16.
47. A genetically engineered plant, comprising an isolated nucleic acid molecule encoding a MYB41 amino acid sequence that is selected from the group consisting of SEQ ID NOs: 38, 40, 44, 46, 49, 52, 70, 76, 77, 78, and 86, operably linked to a nucleic acid sequence encoding a heterologous promoter, wherein expression of the isolated nucleic acid molecule in a plant results in increased levels of suberin as compared to wild-type check plants lacking the isolated nucleic acid molecule.
48. A genetically engineered plant, comprising an isolated nucleic acid molecule that is selected from the group consisting of SEQ ID NOs: 93, 95, 99, 101, 104, 107, 125, 131, 132, 133, 141 148, 150, 154, 156, 159, 162, 180, 186, 187, 188, and 196, operably linked to a nucleic acid sequence encoding a heterologous promoter, wherein expression of the isolated nucleic acid molecule in a plant results in increased levels of suberin as compared to wild-type check plants lacking the isolated nucleic acid molecule.
49. The genetically engineered plant of any one of examples 47 or 48, wherein the plant is canola (Brassica napus), soybean (Glycine max), cotton (Gossypium hirsutum), rice (Oryza sativa), lotus (Lotus japonicus), radish (Raphanus sativus), setaria (Setaria italica), sorghum (Sorghum bicolor), pennycress (Thlaspi arvense), southern cattail (Typha domingensis), wheat (Triticum aestivum), or maize (Zea mays).
50. The genetically engineered plant of examples 47 or 48, wherein the heterologous promoter is a promoter of FACT gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.
51. The genetically engineered plant of examples 47 or 48, wherein the heterologous promoter is a promoter of HORST gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.
52. The genetically engineered plant of examples 47 or 48, wherein the heterologous promoter is a promoter of ASFT gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.
53. The genetically engineered plant of examples 47 or 48, wherein the heterologous promoter is a promoter of GPAT5 gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.
54. The genetically engineered plant of examples 47 or 48, wherein the heterologous promoter is a promoter of RALPH gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.
55. The genetically engineered plant of examples 47 or 48, wherein the heterologous promoter is a promoter of MYB84 gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.
56. The isolated nucleic acid molecule of any one of examples 1-5, wherein the heterologous promoter comprises an isolated nucleic acid sequence comprising at least 1000 consecutive nucleotides from SEQ ID NO: 1, SEQ ID NO: 2, SEQ ID NO: 3, SEQ ID NO: 4, SEQ ID NO: 5 or SEQ ID NO: 6.
   MYB Transcription Factors Involved with Suberin Synthesis and Suberization
1. An isolated nucleic acid molecule comprising a nucleic acid sequence encoding a MYB transcription factor whose amino acid sequence has at least 80% sequence identity to any one of SEQ ID NOs: 14 and 22-36 and/or a nucleic acid sequence having at least 80% sequence identity to any one of SEQ ID NOs: 15 and 202-232, operably linked to a nucleic acid sequence encoding a heterologous promoter, wherein expression of the isolated nucleic acid molecule in a plant results in increased levels of suberin as compared to wild-type check plants lacking the isolated nucleic acid molecule.
2. The isolated nucleic acid molecule of example 1, wherein the increased levels of suberin occur by generating additional periderm cells and/or depositing more suberin in existing periderm cells.
3. The isolated nucleic acid molecule of example 1 or example 2, wherein the amino acid sequence is at least 85% identical, at least 90% identical, at least 95% identical, at least 96% identical, at least 97% identical, at least 98% identical and at least 99% identical to any one of SEQ ID NOs: 14 and 22-36.
4. The isolated nucleic acid molecule of example 1 or example 2, wherein the amino acid sequence is any one of SEQ ID NOs: 14 and 22-36.
5. The isolated nucleic acid molecule of any one of examples 1-4, wherein the nucleic acid sequence is any one of SEQ ID NOs: 15 and 202-232.
6. A transformation vector comprising one or more of the nucleic acid molecules of any one of examples 1-5 and 22-28.
7. A method of transforming a plant cell, the method comprising introducing the transformation vector of example 6 into a plant cell, whereby the transformed plant cell produces increased levels of suberin as compared to an untransformed wild-type check plant cell.
8. The method of example 7, further comprising producing transformed plant tissue from the transformed plant cell.
9. The method of example 8, further comprising producing a transformed plantlet from the transformed plant tissue, wherein the transformed plantlet produces increased levels of suberin as compared to untransformed wild-type check plantlets lacking the isolated nucleic acid molecule.
10. The method of example 9, further comprising producing a progeny of the transformed plantlet, wherein the progeny produces increased levels of suberin as compared to untransformed wild-type check plantlets lacking the isolated nucleic acid molecule.
11. The method of example 9 or example 10, further comprising growing the transformed plantlet or progeny of the transformed plantlet into a mature transformed plant, wherein the mature transformed plant produces increased levels of suberin as compared to mature untransformed wild-type checks lacking the isolated nucleic acid molecule.
12. The method of any one of any one of examples 9-11, wherein the increased levels of suberin occur by generating additional periderm cells and/or depositing more suberin in existing periderm cells.
13. The method of any one of any one of examples 9-12, wherein there is minimal or no expression of the nucleic acid molecule in cells that are not associated with normal suberin production.
14. The method of any one of any one of examples 9-13, wherein there is minimal or no expression of the nucleic acid molecule in rosette leaves.
15. The method of any one of examples 11-14, further comprising using the mature transformed plant or clone of the mature transformed plant in a breeding method.
16. The method of example 15, wherein the breeding method comprises selfing or crossing the mature transformed plant or clone of the mature transformed plant.
17. A plant breeding method comprising crossing a first plant comprising a nucleic acid molecule of any one of examples 1-5 and 22-28 with a second plant of the same species and selecting resultant progeny of the cross based on increased levels of suberin as compared to wild-type check plants.
18. The plant breeding method of example 17, further comprising producing clones of the resultant progeny of the cross wherein the clones are selected based on increased levels of suberin as compared to wild-type check plants.
19. The plant breeding method of example 17 or example 18, wherein the progeny of the cross that display increased levels of suberin as compared to wild-type check plants are selected using molecular markers that are designed based on the nucleic acid molecule of any one of examples 1-5 and 22-28.
20. The method of example 17, further comprising using the selected progeny in a breeding method.
21. The method of any one of examples 11-14, further comprising growing the mature transformed plant or clone of the mature transformed plant in a greenhouse or outdoors.
22. The isolated nucleic acid molecule of any one of examples 1-5, wherein the heterologous promoter comprises an isolated nucleic acid sequence set forth in any one of SEQ ID NO: 1, SEQ ID NO: 2, SEQ ID NO: 3, SEQ ID NO: 4, SEQ ID NO: 5 and SEQ ID NO: 6, or a sequence at least 70%, at least 80%, at least 90%, at least 95%, at least 98%, or at least 99% identical thereto.
23. The isolated nucleic acid molecule of any one of examples 1-5, wherein the heterologous promoter is a promoter of FACT gene.
24. The isolated nucleic acid molecule of any one of examples 1-5, wherein the heterologous promoter is a promoter of HORST gene.
25. The isolated nucleic acid molecule of any one of examples 1-5, wherein the heterologous promoter is a promoter of ASFT gene.
26. The isolated nucleic acid molecule of any one of examples 1-5, wherein the heterologous promoter is a promoter of GPAT5 gene.
27. The isolated nucleic acid molecule of any one of examples 1-5, wherein the heterologous promoter is a promoter of RALPH gene.
28. The isolated nucleic acid molecule of any one of examples 1-5, wherein the heterologous promoter is a promoter of MYB84 gene.
29. The isolated nucleic acid molecule of example 1, wherein the MYB transcription factor is arabidopsis MYB93, arabidopsis MYB122, arabidopsis MYB107, arabidopsis MYB67, arabidopsis MYB84, arabidopsis MYB74, arabidopsis MYB6, arabidopsis MYB39, arabidopsis MYB102, arabidopsis MYB41, arabidopsis MYB4, arabidopsis MYB92, arabidopsis MYB9, arabidopsis MYB36, arabidopsis MYB53, or arabidopsis MYB68.
30. A plant, a part thereof, or a cell thereof, comprising the isolated nucleic acid molecule of any one of examples 1-5 and 22-29.
31. A plant, a part thereof, or a cell thereof, comprising the transformation vector of example 6.
32. A plant, a part thereof, or a cell thereof, produced by the method of any one of examples 7-16.
33. A genetically engineered plant, comprising an isolated nucleic acid molecule encoding a MYB transcription factor whose amino acid sequence that is selected from the group consisting of SEQ ID NOs: 14 and 22-36, operably linked to a nucleic acid sequence encoding a heterologous promoter, wherein expression of the isolated nucleic acid molecule in a plant results in increased levels of suberin as compared to wild-type check plants lacking the isolated nucleic acid molecule.
34. A genetically engineered plant, comprising an isolated nucleic acid molecule that is selected from the group consisting of SEQ ID NOs: 15 and 202-203, operably linked to a nucleic acid sequence encoding a heterologous promoter, wherein expression of the isolated nucleic acid molecule in a plant results in increased levels of suberin as compared to wild-type check plants lacking the isolated nucleic acid molecule.
35. The genetically engineered plant of examples 33 or 34, wherein the heterologous promoter is a promoter of FACT gene, which is operably linked to the isolated nucleic acid molecule encoding a MYB transcription factor whose amino acid sequence that is selected from the group consisting of SEQ ID NOs: 14 and 22-36.
36. The genetically engineered plant of example 35, wherein the heterologous promoter comprises an isolated nucleic acid sequence set forth in any one of SEQ ID NOs: 1, 234, 239-246, 280-285, 314-316, 340-349, 379, 380, and 389-391, or a sequence at least 70%, at least 80%, at least 90%, at least 95%, at least 98%, or at least 99% identical thereto.
37. The genetically engineered plant of examples 33 or 34, wherein the heterologous promoter is a promoter of HORST gene, which is operably linked to the isolated nucleic acid molecule encoding a MYB transcription factor whose amino acid sequence that is selected from the group consisting of SEQ ID NOs: 14 and 22-36.
38. The genetically engineered plant of example 37, wherein the heterologous promoter comprises an isolated nucleic acid sequence set forth in any one of 2, 236, 254-258, 288, 289, 319-321, 352-354, 383, 394, and 395, or a sequence at least 70%, at least 80%, at least 90%, at least 95%, at least 98%, or at least 99% identical thereto.
39. The genetically engineered plant of examples 33 or 34, wherein the heterologous promoter is a promoter of ASFT gene, which is operably linked to the isolated nucleic acid molecule encoding a MYB transcription factor whose amino acid sequence that is selected from the group consisting of SEQ ID NOs: 14 and 22-36.
40. The genetically engineered plant of example 39, wherein the heterologous promoter comprises an isolated nucleic acid sequence set forth in any one of 3, 233, 239-244, 280-285, 314-316, 340-349, 378, 379, and 389-391, or a sequence at least 70%, at least 80%, at least 90%, at least 95%, at least 98%, or at least 99% identical thereto.
41. The genetically engineered plant of examples 33 or 34, wherein the heterologous promoter is a promoter of GPAT5 gene, which is operably linked to the isolated nucleic acid molecule encoding a MYB transcription factor whose amino acid sequence that is selected from the group consisting of SEQ ID NOs: 14 and 22-36.
42. The genetically engineered plant of example 41, wherein the heterologous promoter comprises an isolated nucleic acid sequence set forth in any one of 4, 235, 247-253, 286, 287, 317, 318, 350, 351, 381, 382, 392, and 393, or a sequence at least 70%, at least 80%, at least 90%, at least 95%, at least 98%, or at least 99% identical thereto.
43. The genetically engineered plant of examples 33 or 34, wherein the heterologous promoter is a promoter of RALPH gene, which is operably linked to the isolated nucleic acid molecule encoding a MYB transcription factor whose amino acid sequence that is selected from the group consisting of SEQ ID NOs: 14 and 22-36.
44. The genetically engineered plant of example 43, wherein the heterologous promoter comprises an isolated nucleic acid sequence set forth in any one of 5, 238, 387, and 388, or a sequence at least 70%, at least 80%, at least 90%, at least 95%, at least 98%, or at least 99% identical thereto.
45. The genetically engineered plant of examples 33 or 34, wherein the heterologous promoter is a promoter of MYB84 gene, which is operably linked to the isolated nucleic acid molecule encoding a MYB transcription factor whose amino acid sequence that is selected from the group consisting of SEQ ID NOs: 14 and 22-36.
46. The genetically engineered plant of example 45, wherein the heterologous promoter comprises an isolated nucleic acid sequence set forth in any one of 6, 237, 259-279, 290-313, 322-339, 355-377, 384-386, and 396-411, or a sequence at least 70%, at least 80%, at least 90%, at least 95%, at least 98%, or at least 99% identical thereto.

### INCORPORATION BY REFERENCE

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### REFERENCES

Vishwanath et al., Suberin: biosynthesis, regulation, and polymer assembly of a protective extracellular barrier. Plant cell reports 34, 573-586 (2015).
Carrington et al., Biochemical changes across a carbon saturation gradient: Lignin, cutin, and suberin decomposition and stabilization in fractionated carbon pools. Soil Biology and Biochemistry 47, 179-190 (2012).
Feng & Simpson Molecular-level methods for monitoring soil organic matter responses to global climate change. Journal of environmental monitoring: JEM 13, 1246-1254 (2011).
Preston et al., 13C nuclear magnetic resonance spectroscopy with cross-polarization and magic-angle spinning investigation of the proximate-analysis fractions used to assess litter quality in decomposition studies. Canadian Journal of Botany 75 (1997).
Winkler et al., Insoluble alkyl carbon components in soils derive mainly from cutin and suberin. Organic Geochemistry 36, 519-529, (2005).
Mahmood et al. Overexpression of ANAC046 Promotes Suberin Biosynthesis in Roots of Arabidopsis thaliana. International journal of molecular sciences 20 (2019).
Höfer et al. The Arabidopsis cytochrome P450 CYP86A1 encodes a fatty acid omega-hydroxylase involved in suberin monomer biosynthesis. Journal of experimental botany 59, 2347-2360 (2008).
Wei et al. Three Transcription Activators of ABA Signaling Positively Regulate Suberin Monomer Synthesis by Activating Cytochrome P450 CYP86A1 in Kiwifruit. Frontiers in Plant Science 10, 1650 (2020).
Kosma et al., Identification of an Arabidopsis Fatty Alcohol: Caffeoyl-Coenzyme A Acyltransferase Required for the Synthesis of Alkyl Hydroxycinnamates in Root Waxes. Plant Physiology 160, 237 (2012).
Molina et al., Identification of an Arabidopsis Feruloyl-Coenzyme A Transferase Required for Suberin Synthesis. Plant Physiology 151, 1317, (2009).
Kosma et al. AtMYB41 activates ectopic suberin synthesis and assembly in multiple plant species and cell types. Plant J 80, 216-229, (2014).

1. An isolated nucleic acid molecule comprising a nucleic acid sequence encoding a MYB41 amino acid sequence with at least 80% sequence identity to any one of SEQ ID NOs: 37-91 and/or a nucleic acid sequence having at least 80% sequence identity to any one of SEQ ID NOs: 92-201, operably linked to a nucleic acid sequence encoding a heterologous promoter, wherein expression of the isolated nucleic acid molecule in a plant results in increased levels of suberin as compared to wild-type check plants lacking the isolated nucleic acid molecule.

2. The isolated nucleic acid molecule of claim 1, wherein the increased levels of suberin occur by generating additional periderm cells and/or depositing more suberin in existing periderm cells.

3. The isolated nucleic acid molecule of claim 1, wherein the amino acid sequence is selected from the group consisting of at least 85% sequence identity, at least 90% sequence identity, at least 95% sequence identity, at least 96% sequence identity, at least 97% sequence identity, at least 98% sequence identity and at least 99% sequence identity to any one of SEQ ID NOs: 37-91.

4. The isolated nucleic acid molecule of claim 1, wherein the amino acid sequence is any one of SEQ ID NOs: 37-91.

5. The isolated nucleic acid molecule of claim 1, wherein the heterologous promoter comprises:
an isolated nucleic acid sequence set forth in any one of SEQ ID NO: 1, SEQ ID NO: 2, SEQ ID NO: 3, SEQ ID NO: 4, SEQ ID NO: 5, and SEQ ID NO: 6, or a sequence at least 70% identical thereto; or
an isolated nucleic acid sequence set forth in any one of SEQ ID NOs: 233-411, or a sequence at least 70%, at least 80%, at least 90%, at least 95%, at least 98%, or at least 99% identical thereto.

6. (canceled)

7. A transformation vector comprising one or more of the nucleic acid molecules of claim 1.

8. A method of transforming a plant cell, comprising introducing the transformation vector of claim 7 into a plant cell, thereby generating a transformed plant cell, whereby the transformed plant cell produces increased levels of suberin as compared to an untransformed wild-type check plant cell.

9. The method of claim 8, further comprising producing transformed plant tissue from the transformed plant cell.

10. The method of claim 9, further comprising producing a transformed plantlet from the transformed plant tissue, wherein the transformed plantlet produces increased levels of suberin as compared to untransformed wild-type check plantlets lacking the isolated nucleic acid molecule.

11. The method of claim 10, further comprising producing a progeny of the transformed plantlet, wherein the progeny produces increased levels of suberin as compared to untransformed wild-type check plantlets lacking the isolated nucleic acid molecule.

12. The method of claim 10, further comprising growing the transformed plantlet or the progeny of the transformed plantlet into a mature transformed plant, wherein the mature transformed plant produces increased levels of suberin as compared to mature untransformed wild-type checks lacking the isolated nucleic acid molecule.

13. (canceled)

14. The method of claim 10, wherein there is minimal or no expression of the nucleic acid molecule in cells that are not associated with normal suberin production.

15. The method of claim 10, wherein there is minimal or no expression of the nucleic acid molecule in rosette leaves.

16. The method of claim 12, further comprising using the mature transformed plant or clone of the mature transformed plant in a breeding method.

17. (canceled)

18. A plant breeding method, comprising crossing a first plant comprising a nucleic acid molecule of claim 1 with a second plant of the same species and selecting resultant progeny of the cross based on increased levels of suberin as compared to wild-type check plants.

19.-22. (canceled)

23. The isolated nucleic acid molecule of claim 1, wherein the heterologous promoter is:
a promoter of FACT gene, such as an isolated nucleic acid sequence set forth in any one of SEQ ID NOs: 1, 234, 239-246, 280-285, 314-316, 340-349, 379, 380, or 389-391, or a sequence at least 70%, at least 80%, at least 90%, at least 95%, at least 98%, or at least 99% identical thereto;
a promoter of HORST gene, such as an isolated nucleic acid sequence set forth in any one of SEQ ID NOs: 2, 236, 254-258, 288, 289, 319-321, 352-354, 383, 394, or 395, or a sequence at least 70%, at least 80%, at least 90%, at least 95%, at least 98%, or at least 99% identical thereto;
a promoter of ASFT gene, such as an isolated nucleic acid sequence set forth in any one of SEQ ID NOs: 3, 233, 239-244, 280-285, 314-316, 340-349, 378, 379, or 389-391, or a sequence at least 70%, at least 80%, at least 90%, at least 95%, at least 98%, or at least 99% identical thereto;
a promoter of GPAT5 gene, such as an isolated nucleic acid sequence set forth in any one of SEQ ID NOs: 4, 235, 247-253, 286, 287, 317, 318, 350, 351, 381, 382, 392, or 393, or a sequence at least 70%, at least 80%, at least 90%, at least 95%, at least 98%, or at least 99% identical thereto
a promoter of RALPH gene, such as an isolated nucleic acid sequence set forth in any one of SEQ ID NOs: 5, 238, 387, or 388, or a sequence at least 70%, at least 80%, at least 90%, at least 95%, at least 98%, or at least 99% identical thereto; or
a promoter of MYB84 gene, such as an isolated nucleic acid sequence set forth in any one of SEQ ID NOs: 6, 237, 259-279, 290-313, 322-339, 355-377, 384-386, or 396-411, or a sequence at least 70%, at least 80%, at least 90%, at least 95%, at least 98%, or at least 99% identical thereto.

24.-28. (canceled)

29. The isolated nucleic acid molecule of claim 1, wherein:
the MYB41 amino acid sequence is present in Canola (Brassica napus) and comprises any one of SEQ ID NOs: 37-48 or a sequence at least 80%, at least 90%, at least 95%, at least 98%, or at least 99% identical thereto;
the MYB41 amino acid sequence is present in Soybean (Glycine max) and comprises any one of SEQ ID NOs: 49-56 or a sequence at least 80%, at least 90%, at least 95%, at least 98%, or at least 99% identical thereto;
the MYB41 amino acid sequence is present in Cotton (Gossypium hirsutum) and comprises any one of SEQ ID NOs: 57-68 or a sequence at least 80%, at least 90%, at least 95%, at least 98%, or at least 99% identical thereto;
the MYB41 amino acid sequence is present in Rice (Oryza sativa) and comprises any one of SEQ ID NO: 69 or a sequence at least 80%, at least 90%, at least 95%, at least 98%, or at least 99% identical thereto;
the MYB41 amino acid sequence is present in Lotus (Lotus japonicus) and comprises any one of SEQ ID NOs: 70-72 or a sequence at least 80%, at least 90%, at least 95%, at least 98%, or at least 99% identical thereto;
the MYB41 amino acid sequence is present in Radish (Raphanus sativus) and comprises any one of SEQ ID NOs: 73-81 or a sequence at least 80%, at least 90%, at least 95%, at least 98%, or at least 99% identical thereto;
the MYB41 amino acid sequence is present in Radish (Raphanus sativus) and comprises any one of SEQ ID NOs: 73-81 or a sequence at least 80%, at least 90%, at least 95%, at least 98%, or at least 99% identical thereto;
the MYB41 amino acid sequence is present in Setaria (Setaria italica) and comprises any one of SEQ ID NO: 82 or a sequence at least 80%, at least 90%, at least 95%, at least 98%, or at least 99% identical thereto;
wherein the MYB41 amino acid sequence is present in Sorghum (Sorghum bicolor) and comprises any one of SEQ ID NO: 83 or a sequence at least 80%, at least 90%, at least 95%, at least 98%, or at least 99% identical thereto;
the MYB41 amino acid sequence is present in Pennycress (Thlaspi arvense) and comprises any one of SEQ ID NOs: 84-86 or a sequence at least 80%, at least 90%, at least 95%, at least 98%, or at least 99% identical thereto;
the MYB41 amino acid sequence is present in Southern cattail (Typha domingensis) and comprises any one of SEQ ID NO: 87 or a sequence at least 80%, at least 90%, at least 95%, at least 98%, or at least 99% identical thereto;
the MYB41 amino acid sequence is present in Wheat (Triticum aestivum) and comprises any one of SEQ ID NOs: 88-89 or a sequence at least 80%, at least 90%, at least 95%, at least 98%, or at least 99% identical thereto;
the MYB41 amino acid sequence is present in Maize (Zea mays) and comprises any one of SEQ ID NOs: 90-91 or a sequence at least 80%, at least 90%, at least 95%, at least 98%, or at least 99% identical thereto; or
the MYB41 amino acid sequence comprises at least 80%, at least 90%, at least 95%, at least 98%, or at least 99% sequence identity to any one of SEQ ID NOs: 38, 40, 44, 46, 49, 52, 70, 76, 77, 78, and 86.

30.-41. (canceled)

42. The isolated nucleic acid molecule of claim 1, wherein the nucleic acid sequence comprises:
at least 80%, at least 90%, at least 95%, at least 98%, or at least 99% sequence identity to any one of SEQ ID NOs: 93, 95, 99, 101, 104, 107, 125, 131, 132, 133, and 141; or
at least 80%, at least 90%, at least 95%, at least 98%, or at least 99% sequence identity to any one of SEQ ID NOs: 148, 150, 154, 156, 159, 162, 180, 186, 187, 188, and 196.

43. (canceled)

44. A plant, a part thereof, or a cell thereof, comprising the isolated nucleic acid molecule of claim 1.

45. A plant, a part thereof, or a cell thereof, comprising the transformation vector of claim 7.

46. A plant, a part thereof, or a cell thereof, produced by the method of claim 8.

47. A genetically engineered plant, comprising an isolated nucleic acid molecule encoding a MYB41 amino acid sequence that is selected from the group consisting of SEQ ID NOs: 38, 40, 44, 46, 49, 52, 70, 76, 77, 78, and 86, operably linked to a nucleic acid sequence encoding a heterologous promoter, wherein expression of the isolated nucleic acid molecule in a plant results in increased levels of suberin as compared to wild-type check plants lacking the isolated nucleic acid molecule.

48. A genetically engineered plant, comprising an isolated nucleic acid molecule that is selected from the group consisting of SEQ ID NOs: 93, 95, 99, 101, 104, 107, 125, 131, 132, 133, 141 148, 150, 154, 156, 159, 162, 180, 186, 187, 188, and 196, operably linked to a nucleic acid sequence encoding a heterologous promoter, wherein expression of the isolated nucleic acid molecule in a plant results in increased levels of suberin as compared to wild-type check plants lacking the isolated nucleic acid molecule.

49. (canceled)

50. The genetically engineered plant of claim 47, wherein the heterologous promoter is a promoter of:
FACT gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86;
HORST gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86;
ASFT gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86;
GPAT5 gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86;
RALPH gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86; or
MYB84 gene, which is operably linked to the isolated nucleic acid molecule encoding a MYB41 amino acid sequence that is selected from the group consisting of SEQ ID NOs: 38, 40, 44, 46, 49, 52, 70, 76, 77, 78, and 86.

51. The genetically engineered plant of claim 50, wherein the heterologous promoter comprises:
an isolated nucleic acid sequence set forth in any one of SEQ ID NOs: 1, 234, 239-246, 280-285, 314-316, 340-349, 379, 380, or 389-391, or a sequence at least 70%, at least 80%, at least 90%, at least 95%, at least 98%, or at least 99% identical thereto
an isolated nucleic acid sequence set forth in any one of SEQ ID NOs: 2, 236, 254-258, 288, 289, 319-321, 352-354, 383, 394, and 395, or a sequence at least 70%, at least 80%, at least 90%, at least 95%, at least 98%, or at least 99% identical thereto;
an isolated nucleic acid sequence set forth in any one of SEQ ID NOs: 3, 233, 239-244, 280-285, 314-316, 340-349, 378, 379, and 389-391, or a sequence at least 70%, at least 80%, at least 90%, at least 95%, at least 98%, or at least 99% identical thereto;
an isolated nucleic acid sequence set forth in any one of SEQ ID NOs: 4, 235, 247-253, 286, 287, 317, 318, 350, 351, 381, 382, 392, and 393, or a sequence at least 70%, at least 80%, at least 90%, at least 95%, at least 98%, or at least 99% identical thereto;
an isolated nucleic acid sequence set forth in any one of SEQ ID NOs: 5, 238, 387, and 388, or a sequence at least 70%, at least 80%, at least 90%, at least 95%, at least 98%, or at least 99% identical thereto; or
an isolated nucleic acid sequence set forth in any one of SEQ ID NOs: 6, 237, 259-279, 290-313, 322-339, 355-377, 384-386, and 396-411, or a sequence at least 70%, at least 80%, at least 90%, at least 95%, at least 98%, or at least 99% identical thereto.

52.-61. (canceled)
